SEQUENCE LISTING (15 sequences)

SEQ ID NO: 1 - protein, 271 aa, Homo sapiens
MEDSHKSTTSETAPQPGSAVQGAHISHIAQQVSSLSESEESQDSSDSIGSSQKAHGILARRPSYRKILKDLSSEDTRGRKGDGENSGVSAAVTSMSVPTPIYQTSSGQYIAIAPNGALQLASPGTDGVQGLQTLTMTNSGSTQQGTTILQYAQTSDGQQILVPSNQVVVQTASGDMQTYQIRTTPSATSLPQTVVMTSPVTLTSQTTKTDDPQLKREIRLMKNREAARECRRKKKEYVKCLENRVAVLENQNKTLIEELKTLKDLYSNKSV

SEQ ID NO: 2 - protein, 341 aa, Homo sapiens
MTMESGAENQQSGDAAVTEAENQQMTVQAQPQIATLAQVSMPAAHATSSAPTVTLVQLPNGQTVQVHGVIQAAQPSVIQSPQVQTVQSSCKDLKRLFSGTQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSD

SEQ ID NO: 3 - protein, 281 aa, Saccharomyces cerevisiae
MSEYQPSLFALNPMGFSPLDGSKSTNENVSASTSTAKPMVGQLIFDKFIKTEEDPIIKQDTPSNLDFDFALPQTATAPDAKTVLPIPELDDAVVESFFSSSTDSTPMFEYENLEDNSKEWTSLFDNDIPVTTDDVSLADKAIESTEEVSLVPSNLEVSTTSFLPTPVLEDAKLTQTRKVKKPNSVVKKSHHVGKDDESRLDHLGVVAYNRKQRSIPLSPIVPEIDDPAALKRARNTEAARRSRARKLQRMKQLEDKVEELLSKNYHLENEVARLKKLVGER

SEQ ID NO: 4 - DNA, 27 bp, Artificial Sequence
  Oligonucleotide
agagattgcctgacgtcagagagctag

SEQ ID NO: 5 - DNA, 21 bp, Artificial Sequence
  Oligonucleotide
cgcttgatgagtcagccggaa

SEQ ID NO: 6 - DNA, 30 bp, Artificial Sequence
  Primer
gactagatgagagctactctaagaggaacg

SEQ ID NO: 7 - protein, 94 aa, Artificial Sequence
  Protein
QVKLQQSGGGLVQPGGSWKLSCAASGFTFSSEGMHWVRQAPEKGLEWVAYISSGSSTLHYADTVKGRFTISRDNPKNTLFLQMKLPSLCYGLLG

SEQ ID NO: 8 - protein, 107 aa, Artificial Sequence
  Protein
QSPASLAVSLGQRATISCKASQSVDYDGDSYMNWYQQKPGQPPKLLIFGASNLESGIPARFTGSGSGTDFTLNIHPVEEEDAATYYCQQSNEDPFTFGSGTKLEIKR

SEQ ID NO: 9 - protein, 15 aa, Artificial Sequence
  Peptide Internal Fragment
GGGGSGGGGSGGGGS

SEQ ID NO: 10 - protein, 11 aa, Artificial Sequence
  Peptide
XXXXXXKRXXR

SEQ ID NO: 11 - protein, 20 aa, Artificial Sequence
  Peptide
XXXXXXXXXXXKRXXRXXXN

SEQ ID NO: 12 - protein, 31 aa, Artificial Sequence
  Peptide
XXXXXXXXXXXKRXXRXXXNXXARXRKXXXX

SEQ ID NO: 13 - DNA, 14 bp, Artificial Sequence
  Consensus sequence
gcctgacgtcaccg

SEQ ID NO: 14 - protein, 7 aa, Artificial Sequence
  Synthetic Sequence
PKKKRKX

SEQ ID NO: 15 - protein, 8 aa, Artificial Sequence
  Synthetic Sequence
PKKKRKVE

(19) United States
(12) Patent Application Publication
(10) Pub. No.: US 2005/0214309 A1
(43) Pub. Date: Sep. 29, 2005

(54) Methods and compositions for modulating transcription factor activity

(76) Inventors: Steven H. Hinrichs, Omaha, NE (US); Randall J. Olsan, Houston, TX (US); Julia A. Bridge, Omaha, NE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/965,327

(22) Filed: Oct. 14, 2004

Related U.S. Application Data
(63) Continuation of application No. 09/519,665, filed on Mar. 6, 2000, now abandoned.
Application No. 09/519,665 is a continuation-in-part of application No. 08/881,800, filed on Jun. 24, 1997, now abandoned.
Application No. 08/881,800 is a continuation-in-part of application No. 08/210,880, filed on Mar. 18, 1994, now Pat. No. 5,641,486.

(51) Int. Cl. A61K 39/395; A61K 38/47

(52) U.S. Cl. 424/178.1; 424/94.61

(57) ABSTRACT

The present invention relates generally to transcription factor pathways, the modulation of such pathways, agents which modulate the activity of transcription factors, screening molecules to identify transcription factor modulators and cell or animal models for tumor-related transcription factors. More particularly, the present invention relates to the modulation of transcription factors in which the DNA binding domain is distinct from the activation domain by binding an inhibitory agent to a region adjacent to the DNA binding domain.

### CROSS-REFERENCE TO RELATED APPLICATIONS

The present application is a continuation-in-part application of Ser. No. 08/881,800, filed Jun. 24, 1997, which is a continuation-in-part of Ser. No. 08/210,880, filed Mar. 18, 1994, and issued as U.S. Pat. No. 5,641,486 on Jun. 24, 1997, all of which are incorporated herein by reference.

### BACKGROUND OF THE INVENTION

The present invention relates generally to transcription factor pathways, the modulation of such pathways, agents which modulate the activity of transcription factors, the screening of molecules to identify transcription factor modulators and cell or animal models for tumor-related transcription factors. More particularly, the present invention relates to the modulation of transcription factors in which the DNA binding domain is distinct from the activation domain by binding an inhibitory agent to a region adjacent to the DNA binding domain.

The publications and other materials used herein to illuminate the background of the invention, and in particular cases, to provide additional details respecting the practice, are incorporated by reference, and for convenience are referenced in the following text and grouped in the appended bibliography.

Gene expression leading to the production of protein is most frequently regulated at the level of RNA production, which is termed transcription. Generally, control of transcription is mediated by activator or repressor proteins termed transcription factors. A gene is transcribed after a sequence of events determined by transcription factors has resulted in positioning an enzyme (i.e., RNA polymerase) in the proper location and configuration on the DNA. Transcription factors act through at least two essential mechanisms: (i) binding to specific DNA sequences; and (ii) interacting with other proteins which subsequently influence transcription initiation (trans-activation). The activities of transcription factors in binding DNA and activation of transcription are typically controlled by two functionally different regions (domains), one that binds to specific DNA sequences (DNA-binding domains), and another that activates transcription (TAD). Some transcription factors include a dimerization region which may or may not be part of the DNA-binding domain. Other transcription factors do not require dimerization for DNA-binding activity, e.g., homeodomain proteins.

Proteins that regulate transcription of DNA recognize specific sequences through discrete DNA-binding domains within their polypeptide chains. Genes encoding specific transcription factors have been cloned and sequenced. By comparing the deduced amino acid sequences of these proteins it has become apparent that their DNA-binding regions comprise a very limited number of structural motifs. For this reason, transcription factors are often classified according to the type of DNA-binding domain they contain. The DNA-binding domain may be present in either the N-terminal amino acids, for example Gal4 of yeast, or the C-terminal amino acids, for example Gen4 of yeast. The more common DNA-binding motifs include leucine zipper, zinc-finger, forkhead, and helix-loop-helix or homeodomain proteins. A structural model of eukaryotic activating transcription factors has emerged in which one or more TAD is connected to a sequence specific, DNA-binding domain through relatively flexible protein domains.

For example, in the b-ZIP superfamily of transcription factors, the most significant structural similarity is the presence of a region with many basic amino acids (b region), and a separate domain that allows close interaction with other proteins with like structure, analogous to a zipper (ZIP). The basic domain has a high concentration of the positively charged amino acids lysine and arginine, which form a tightly coiled alpha helix in the presence of DNA which facilitates binding to DNA. The basic domain lies in close proximity to a series of amino acids in which leucine is present at every seventh position (the leucine zipper). Further, the leucine zipper forms an amphipathic alpha helix organized into coiled-coils with one surface being hydrophobic and the opposite surface being hydrophilic. This provides for close pairing or dimerization with either identical proteins (homodimers) or similar proteins (heterodimers).

The DNA sequences which are involved in regulation of either viral or eukaryotic gene expression and are the sites for transcription factor regulation occur in a variety of locations and at various distances from the transcriptional start and stop sites. These DNA sequences which contribute to regulation consist of complex arrays of relatively short DNA sequence motifs. It is believed that tissue specific gene expression occurs as a consequence of cooperation between transcription factors and the DNA sequences to which they bind. Each DNA motif is a binding site for a specific family of transcription factors.

For example, in the CREB/ATF1 family, the consensus binding site has been identified by Montminy et al. (1986). This sequence, TGACGTCA, is present in a wide variety of viral and cellular genes, most notably E1A inducible adenoviral genes and cAMP-inducible cellular genes. Some variation is found in the core sequence with retention of essential function. This sequence is capable of being bound by members of the CREB/ATF1 family and, at a lower affinity, by transcription factors in other b-ZIP subfamilies such as the AP-1 components, fos and jun (Sassome-Corsi, et al., 1988). Specificity of CREB protein binding to particular enhancers can be altered by interaction with viral oncoproteins, including Hepatitis B virus X (Maguire, et al., 1991), Human T-cell leukemia virus (HTLV-1) Tax (Zhao et al., 1992; Armstrong et al., 1992; Suzuki, et al., 1993; Wagner and Green, 1993).

Characteristic chromosomal translocations have been identified in leukemias, lymphomas, and sarcomas. These translocations frequently involve genes encoding transcription factors (Ladanyi, 1995; Bridge et al., 1990). The common feature of many translocations is the generation of a chimeric gene resulting in a fusion protein containing portions of both genes involved in the translocation. The combination of specific domains from unrelated transcription factors may result in the generation of chimeric, fusion proteins with activity distinct from either of its components (Bridge et al., 1990). Since the fusion proteins are unique to the tumor cell, they represent a true tumor specific antigen.

Characteristic translocations not only serve as specific markers of each particular tumor type but also are believed to contribute to the underlying mechanism leading to malignancy. Several lines of evidence suggest that the fusion proteins found in various neoplasias play a critical role in development of the transformed phenotype. However, it has not been demonstrated whether chimeric proteins are essential for continued cell proliferation, or whether other processes have developed that are irreversible.

It is desired to further characterize the modulation of transcription factors, to identify inhibitory agents and to identify the role of fusion protein binding to DNA in the neoplastic process. It is also desired to develop phenotypic knockouts of tumor-related proteins as a means to define the mechanism of tumor cell killing and to develop a therapeutic model or prototype of rational drug design.

### SUMMARY OF THE INVENTION

The present invention relates generally to transcription factor pathways, the modulation of such pathways, agents which modulate the activity of transcription factors, the screening of molecules to identify transcription factor modulators and cell or animal models for tumor-related transcription factors. More particularly, the present invention relates to the modulation of transcription factors in which the DNA binding domain is distinct from the activation domain by binding an inhibitory agent to a region adjacent to the DNA binding domain. In one embodiment of the present invention, the transcription factor which can be modulated is a wild-type transcription factor. In a first aspect of this embodiment, the wild-type transcription factor is a B-ZIP transcription factor. In a second aspect of this embodiment, the wild-type transcription factor is a helix-loop-helix protein. In a third aspect of this embodiment, the wild-type transcription factor is a zinc finger transcription factor. In a second embodiment of the present invention, the transcription factor is a mutant protein which has a DNA binding domain and an activation domain distinct from each other. In one aspect of this embodiment, the mutant protein is a chimeric protein which results from a chromosomal translocation, such as a fusion protein.

The present invention also relates generally to the modulation of transcription factor activity. The modulation of transcription factor activity is useful for cancer and antiviral therapy because the transcription factors provide unique targets. In one embodiment of the present invention, the modulation of transcription factor activity is the inhibition of such activity. In one aspect of this embodiment, transcription factor activity is modulated to inhibit transcription factor mediated gene expression. In a second aspect of this embodiment, transcription factor activity is modulated to inhibit transcription factor mediated viral replication. In a third aspect of this embodiment, transcription factor activity is modulated to inhibit transcription factor mediated cellular proliferation. The inhibition of transcription factor activity is preferably accomplished either by inhibiting the DNA binding activity of transcription factors or by dissociation of the transcription factor from the DNA, for example by increasing off-rate of the transcription factor or preventing its rebinding. The DNA binding activity is inhibited by binding an agent, sometimes referred to herein as an inhibitory agent, to a newly identified region on a transcription factor adjacent to the DNA binding domain, sometimes referred to herein as linker domain. It has been discovered that the binding of an inhibitory agent to a transcription factor induces apoptosis.

The present invention further relates to screening molecules to identify compounds which modulate transcription factor activity, e.g., the binding of a transcription factor to DNA.

Finally, the present invention relates to the use of intracellular inhibitory agents to develop phenotypic knockouts of oncogenic fusion proteins as a means to define the mechanism of tumor cell killing and to develop a therapeutic model of rational drug design.

### BRIEF DESCRIPTION OF THE FIGURES

FIG. 1 shows a conceptual model to illustrate how a sFv or related molecule interferes with the activity of transcription factors belonging to the b-ZIP family.

FIG. 2 shows a comparison of a portion of the protein sequences for the b-ZIP transcription factors ATF1, CREB and GCN4.

FIG. 3 shows the results of MAb1 and MAbs 3-5 in immunoblot assays as described in Example 2. (The preparation of these MAbs is described hereinafter.)

FIG. 4 shows the results of the DNA binding assay with the MAb1 and MAbs 3-5 panel and IgA and IgG1 antibodies as described in Example 3.

FIG. 5 shows the promoter templates for the in vitro PCNA transcription studies described in Example 4.

FIG. 6 shows the effects of the MAb on in vitro PCNA transcription as described in Example 4.

FIG. 7 shows the regions of interest on CREB and ATF1.

FIG. 8 shows MAb1 and MAbs3-5 reactivity with major thrombin fragments of recombinant ATF1 as described hereinafter in Example 5.

FIG. 9 shows the DNA binding analysis with thrombin digested ATF1:undigested ATF1 (lane 1), digested ATF1 (lane 2), digested ATF1 with 30×unlabeled CRE competitor (lane 3) or MAb1 and MAbs 3-5 (i.e., M1 and M3-5, lanes 4-7), as described in Example 5.

FIG. 10 shows a graph of peptide c binding of MAb4 by competitive inhibition ELISA as described in Example 6.

FIG. 11 shows the inhibitory nature of the MAb4, FAb4 and sFv4 proteins for either ATF1 or CREB.

FIG. 12 shows the in vivo inhibitory effect of the sFv4 protein on ATF1 and CREB in HeLa and 293T cells.

FIG. 13 shows the inhibitory effect of the sFv4 protein on the activity of the viral HTLV-I Tax protein.

### SUMMARY OF SEQUENCE LISTING

SEQ ID NO:1 is the amino acid sequence of the ATF1 protein.

SEQ ID NO:2 is the amino acid sequence of the CREB protein.

SEQ ID NO: 3 is the amino acid sequence of the GCN4 protein.

SEQ ID NO:4 and 5 are the double-stranded oligonucleotides used in the electrophoretic mobility shift assays.

SEQ ID NO:6 is a ³²P labeled primer.

SEQ ID NO:7 is the consensus amino acid sequence for the V_{H} region of the sFv clones.

SEQ ID NO:8 is the consensus amino acid sequence for the V_{L} region of the sFv clones.

SEQ ID NO:9 is the amino acid sequence of the linker peptide for sFv4.

SEQ ID NO:10 is a consensus sequence for the linker domains of b-ZIP transcription factors.

SEQ ID NO:11 is a consensus sequence for the linker domains of b-ZIP transcription factors.

SEQ ID NO:12 is a consensus sequence for the linker domains of b-ZIP transcription factors.

SEQ ID NO:13 is the nucleotide sequence for the consensus CRE element.

### DETAILED DESCRIPTION OF THE INVENTION

The present invention relates generally to transcription factor pathways, the modulation of such pathways, agents which modulate the activity of transcription factors, the screening of molecules to identify transcription factor modulators and cell or animal models for tumor-related transcription factors. More particularly, the present invention relates to the modulation of transcription factors in which the DNA binding domain is distinct from the activation domain by binding an inhibitory agent to a region adjacent to the DNA binding domain.

The various embodiments of the present invention described herein are based on the discovery of a linker domain on transcription factors which can be used to modulate, more specifically to inhibit, the binding of the transcription factor to DNA or to dissociate the transcription factor from the DNA. For example, the linker domain was first identified on the transcription factor ATF1, a member of the b-ZIP superfamily of transcription factors, from an analysis of the binding of an ATF1 specific antibody which inhibited ATF1 binding to DNA. Similar linker domains have also been found in other b-Zip transcription factors, such as CREB and GCN4. Analysis of other families of transcription factors have identified similar linker domains on wild type helix-loop-helix transcription factors, and on oncogenic fusion proteins which function as transcription factors, such as EWS/ATF1, EWS/FLI1 and PAX/FKHR. On the basis of these linker domains, compounds have been identified which will inhibit transcription factor activity. It has been found that compounds which inhibit one transcription factor of a family, will inhibit other transcription factors of the same family. It has also been found that other families of transcription factors have similar linker domains and that, in the same manner, compounds can be identified which inhibit the activity of these other transcription factors.

Consequently, an embodiment of this invention constitutes an inhibitory agent which binds to a transcription factor for a target gene, with sufficient binding affinity to cause disassociation of the transcription factor from the DNA of the target gene, for example, by increasing the off-rate of the transcription factor or preventing its rebinding and/or otherwise inhibit the transcription factor from binding thereto. As a result, transcription is prevented or at least inhibited, resulting in events of consequence to a virus or cell. The inhibitory agent can be an antibody, subcomponents of the antibody (e.g., Fab fragments or sFv subunits), a polypeptide representing the configuration of the antibody binding site (peptide mimetic), or small molecules that also resemble the configuration of the antibody binding site, for example, a glycopeptide (non-peptide mimetic); provided that in each case the inhibitory agent is capable of binding specifically to the intended linker domain on the transcription factor (e.g., the linker domain of ATF1, CREB or GCN4 previously discussed) with consequent prevention or inhibition of transcription. It is preferred, especially when the inhibitory agent is to be used as a therapeutic agent, that the inhibitory agent target a region of said fragment having no more than about 8 amino acids because a smaller compound is more stable, is more capable of entering cells, and has reduced side effects.

Three plausible explanations for the effect of the inhibitory agents of the invention have been considered. Either the inhibitory agent binds transcription factor in the nucleus to prevent its subsequent binding to DNA in a steric or allosteric manner, or it binds transcription factor in the cytoplasm leading to its immunodepletion or premature degradation. Alternatively, the inhibitory agent may enter the nucleus already bound to transcription factor. It has been discovered that sFv4, an exemplary inhibitory agent of the invention, localizes to the nucleus and binds EWS/ATF1. Consequently, one aspect of this embodiment is an inhibitory agent which enters the nucleus and modulates activity of a transcription factor.

Another embodiment of the present invention relates to a method for preventing, ex vivo or in vivo, transcription factor mediated replication of cancer cells or viruses or for the induction of apoptosis, comprising exposing said cells or viruses, ex vivo or in vivo, to an effective amount of an inhibitory agent of this invention. Said agent binds to a portion of the transcription factor with sufficient binding affinity to cause disassociation of the transcription factor from the DNA of the target gene and/or prevent binding of the transcription factor to DNA and thereby modulate transcription.

Another embodiment is a method for modulating transcription factor binding to cellular DNA, comprising exposure of said DNA to an effective amount of the inhibitory agent which binds to a portion of the transcription factor and disrupts or inhibits binding of the transcription factor to DNA, inhibiting or modulating transcription. One aspect of this embodiment is a method for disassociating transcription factors from DNA, comprising exposing said DNA to an effective amount of an inhibitory agent which specifically binds to a portion of the transcription factor, for example a b-ZIP factor, and results in disassociation.

A further embodiment of the present invention is a method for achieving a phenotypic knockout of tumor-related proteins, comprising expression of intracellular inhibitory agents, and a related method for determining the function of the tumor-related protein. In one aspect of this embodiment, and as is demonstrated hereinafter, the inhibitory compound of this invention can be a monoclonal antibody or a subcomponent of a monoclonal antibody. Exemplary of such subcomponents are Fab fragments or sFv subunits of the monoclonal antibody. The sFv element is thought to be the smallest component of an antibody that is capable of binding to the original epitope and derived sFv proteins have been shown to have binding affinities equivalent to the parent monoclonal antibody (Bird et al., 1988).

Inhibition using monoclonal antibodies (MAb) has been demonstrated. The use of antibodies as transcription factor blocking agents is particularly attractive because the affinity of their binding can easily exceed that of transcription factors for DNA; typically in the nM or M range (Anderson and Dynan, 1994). Exemplary of such MAb is MAb4, hereinafter described. However, because of its size, a MAb is not an ideal inhibitory therapeutic agent. Consequently, it is preferred to use subcomponents of the MAb or, alternatively, to employ a small peptide or other small molecule which binds to the linker domain of the transcription factor. Exemplary of such domain is the ATF1 epitope depicted by residues 205-219 of SEQ ID NO:1.

sFv's have been used in a variety of applications including development of diagnostic, and pharmaceutical compounds. Intracellular expression of sFv's, also referred to as intracellular immunization has been used to disrupt the activity of specific viral genes and to explore the functional role of cellular gene products (Richardson et al., 1995). Several recent reports describe the use of intracellular sFv's to inhibit the activity of various HIV specific enzymes and to explore mechanistic questions related to viral replication (Levy-Mintz et al., 1996). Targeting of transcription factors as an approach to treating cancer was unexpected because the site of activity for transcription factors is the nucleus of a cell. Antibodies generally are not able to enter a cell if they are made outside of a cell, and it is generally believed that if an antibody is made inside of a cell it will be transported to the cell surface and released. A second expectation is that the antibody will stay in the cytoplasm if it is made in the cell. It has been discovered, as described herein, that the sFvs of the invention is able to get into the nucleus and block activity of the transcription factor. Quite unexpectedly, the sFvs are capable of moving into the nucleus and block activity of the transcription factors.

In another aspect of this embodiment, the inhibitory agents of this invention, for example sFvs, are capable of entering the nucleus and inhibiting activity of transcription factors. Prior uses of sFvs have been limited to the cell surface and cytoplasm. The targeting of transcription factors with sFvs as an approach to treating cancer was unexpected. This is partly because the site of activity of transcription factors is the nucleus and there have been no reports, to date, of sFvs that enter the nucleus. Furthermore, sfvs are not believed to be processed like natural separate heavy and light chain proteins nor to contain sequences for cytoplasmic membrane localization and release.

The linker domain of ATF1 has been determined to be a peptide fragment, spanning from about position 205 to about position 219 of the amino acid sequence of the ATF1 protein (SEQ ID NO:1). This fragment is located adjacent to the DNA binding region of ATF1, and is composed of the following amino acid sequence:

(residues 205 to 219 of SEQ ID NO:1)

Gln Thr Thr Lys Thr Asp Asp Pro Gln Leu Lys Arg
Glu Ile Arg

The linker domain of CREB has been determined to be a peptide fragment, spanning from about position 275 to about position 289 of the amino acid sequence of the CREB protein (SEQ ID NO:2). This fragment is located adjacent to the DNA binding region of CREB, and is composed of the following amino acid sequence:

(residues 275 to 289 of SEQ ID NO:2)

Pro Thr Gln Pro Ala Glu Glu Ala Ala Arg Lys Arg
Glu Val Arg

The linker domain of GCN4 has been determined to be a peptide fragment, spanning from about position 224 to about position 234 of the amino acid sequence of the GCN4 protein (SEQ ID NO:3). This fragment is located adjacent to the DNA binding region of GCN4, and is composed of the following amino acid sequence:

(residues 224 to 234 of SEQ ID NO:3)

Ile Asp Asp Pro Ala Ala Leu Lys Arg Ala Arg

On the basis of these sequences, the following consensus sequences have been derived:

(1)

(SEQ ID NO:10)

(X₁)₂—X₂—X₃—K—R—X₄—R;
(2)

(SEQ ID NO:11)

X₀—(X₁)₂—X₃—K—R—X₄—R—X₅—N;
and
(3)

(SEQ ID NO:12)

X₀—(X₁)₂—X₂—X₃—K—R—X₄—R—X₅—N—X₆—X₇—A—R—X₇—R—K—X₈,

wherein
X₀ is 1-5 amino acids,
X₁ is an acidic amino acid,
X₂ is 2-3 amino acids,
X₃ is L or R,
X₄ is 1-2 amino acids,
X₅ is 0-3 amino acids,
X₆ is 1 amino acid,
X₇ is E-A,
X₈ is 3-4 amino acids, and
X₉ is 0-2 amino acids.

The evidence presented herein derived from using ATF1 as a representative of the ATF/CREB family of transcription factors has application to other members of the b-ZIP superfamily, and to other families of transcription factors, including those in which dimerization is not important for binding activity. The region of interest in the ATF1 transcription factor (containing the epitope of mAb4) resides within a structural domain that is a transition region between the DNA binding region (which represents about one-quarter of the protein) and the TAD or activation domain (which represents about three-quarters of the protein). While the mechanism of inhibition by sFv4 is not fully understood, with CREB and ATF-1 already bound to DNA, the mechanism of inhibition may be through allosteric mechanisms that induce a conformational change in a linker domain of the transcription factor or by disrupting residue side chains interactions with the phosphate-DNA backbone, destabilizing the interaction. Alternatively, the mechanism may be steric hindrance of the ATF-1-DNA interaction, with the antibody blocking binding of transcription factor to DNA by occupying a region adjacent to the DNA binding domain. Since the off rate of CREB and ATF1 from DNA is known to be rapid (Anderson et al., 1994) the presence of sFv4 in the region between the helices may prevent rebinding of the factor to DNA and/or increase the off-rate of the factor. The essential issue is that the transition region between functional domains of a transcription factor is comprised of a protein fragment which is sometimes referred to herein as a linker domain. (The term domain is used here beyond its traditional use in defining a region with functional activity.) Connection of one functional domain to another represents the functional activity of these linker domains. Linker domain is common to all transcription factors with DNA binding domains distinct from activation domains. Exemplary of these linker domains are the sequences for members of the b-ZIP family which appear to be distinct within this domain, but each protein contains such a transition region where the alpha helix structure is terminated. While the epitope for ATF1, CREB and GCN4 have been utilized in the discovery of the novel inhibitory agents for such transcription factors, inhibitory agents of the invention can be screened for other transcription factors using the TFDA assay of the present invention, as is more fully described hereinafter. In the present invention, the common features are represented by 1.) the transcription activation domain (TAD); 2.) the DNA binding domain and optional dimerization region; and 3.) the linker domain containing the unique sequence which, in the example of ATF1, CREB and GCN4, has been determined to be the epitope of mAb41.4. Examination of the protein sequences for 37 members of the b-ZIP family in the region adjacent to the DNA binding domain, reveals that the putative linker domains are highlighted in terms of their uniqueness and their position between the b-ZIP domains and the TAD (Biosilevac, et al., in press). Not only are linker domains present in b-ZIP transcription factors, but they are present in other families of transcription factors, for example, helix-turn-helix proteins and zinc finger proteins. Therefore the demonstration of linker domains as targets for inhibitory agents is significant and has broad application. With knowledge of the structural features of transcription factors, and applying the approach which was demonstrated with the b-ZIP superfamily of transcription factors, additional inhibitory agents effective with other families of transcription factors have been developed.

Biophysical and structural properties of sFvs. Examination of the biophysical and structural properties of the inhibitory sFvs of the invention aids in understanding the mechanism involved in the inhibitory process and in the design of improved inhibitory molecules. The molecular mechanism involved in disruption of transcription factor activity can be investigated through kinetic binding studies, structural analysis and mutagenesis of the inhibitor sFv. Such studies can reveal whether the binding of a given antibody to its transcription factor is competitive, involving steric hindrance or non-competitive, involving conformational changes of the targeted transcription factors. Exemplary of this approach, the kinetic and equilibrium parameters underlying the reactivity of mAb4 IgG and its derivative sFv with the transcription factors can be established through studies of the ternary interaction between antibodies (mAb4 IgG and sFv4), DNA binding proteins and DNA (Example 12).

However, it is not necessary to define affinity and rate for all of the potential interactions that occur between DNA, the transcription factor, and the inhibitory antibody mAb4. Whether the inhibitory property of mAb4 results from binding to ATF1while bound to DNA, or while ATF1 is in the off state can be ascertained through studies that confirm or negate the hypothesis that the sFv or Fab interacts with ATF1 when it is not bound to DNA, and that the ATF1/antibody complex is not able to bind to DNA. However, the rate of ATF1 dimerization, and DNA-ATF1 complex formation remains of importance as these factors may influence the inhibitory process. The approach is made easier by the natural presence of tryptophan residues in sFv4 and their absence in ATF1. The determination of baseline affinity constants of the mAb and sFv for ATF1 can be used to establish the mechanism and provide comparison with affinity constants of improved sFv's.

The powerful technique of fluorescence resonance energy transfer (FRET) measures conformational changes, association and dissociation rates, and binding constants down to nM and lower (Parkhurst and Parkhurst, 1994; Parkhurst, et al., 1996; Parkhurst and Parkhurst, 1995(a); Parkhurst and Parkhurst, 1995(b)). Utilizing FRET, changes either in the steady-state or in the time domain can be used to measure binding constants. When employed along with stopped-flow methods, one can obtain rate constants for association and dissociation processes can be obtained (Parkhurst, et al., 1996) (Example 12). Details of this and alternative methods are known in the art (Parkhurst and Parkhurst, 1995(a); Parkhurst and Parkhurst, 1995(b); Bose et al., 1997; and Schreiber and Parkhurst, 1984). Determination of such first-order processes can give insight into the origins of tight binding that is sequence dependent, for instance in the interactions of TBP (TATA binding protein) and specific DNA sequences (Parkhurst et al, in preparation). Additionally, these data provide biophysical evidence for the mechanism of action of the sFv and provide support for the rational design of sFv's which selectively bind to transcription factors.

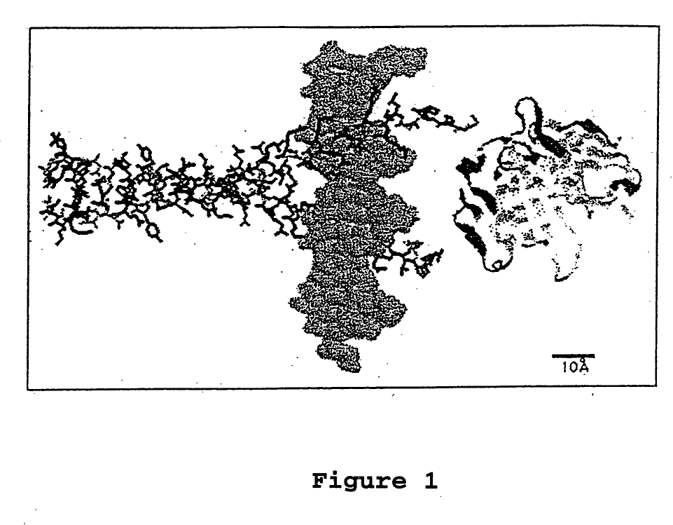

Model of inhibition by sFvs. A conceptual model to illustrate how the sFv or a related compound interferes with the activity of transcription factors belonging to the b-ZIP family has been developed and is illustrated in FIG. 1. Members of the b-ZIP superfamily of transcription factors are defined by the presence of several key features including the formation of dimers and a special structural element known as a b-ZIP domain, which is composed of basic (hence b) amino acids and a second region containing leucine residues spaced at uniform intervals. These leucines provide for interaction with the second molecule which allows for dimerization, likened to the action of a zipper (hence ZIP). The b-ZIP factors appear to have evolved from common ancestral genes (Meyer and Habener, 1993) and are known to regulate transcription of a wide variety of genes. Meyer and Habener (1993) have published detailed comparisons of a large number of cloned b-ZIP transcription factors, and have shown that the activation domains (which may compose 75% of the protein) have a variety of sequence and structure, the b-ZIP domain is unusually similar. One embodiment of this invention relates to the region adjacent to the DNA-binding domain of the transcription factor. For example, for the b-ZIP domain, this region begins 3 residues amino to the invariant asparagine found in all b-ZIP proteins and extending 18 residues amino to the invariant asparagine. This region is characterized as a zone of transition from the highly conserved sequences found in the DNA binding domain to the highly variable sequences found in the region containing the first proline amino to the invariant asparagine.

The example in FIG. 1 illustrates how one aspect of an embodiment of this invention is capable of inhibiting not only ATF1, but other B-ZIP transcription factors as well. This model explains the reactivity of the sFv with CREB (as shown by the Examples), that was not seen with the original monoclonal antibody as described in the Examples. This model illustrates a mechanism for the ability of the inhibitory agent(s) to disrupt transcription as normally exists through binding of any b-ZIP transcription factor to DNA. Further, the model demonstrates how the activity of viral proteins may be disrupted as discussed in Example 11 with the specific capability of the sFv to inhibit the viral HTLV Tax enhancement of transcriptional activation by b-ZIP proteins responsible for the development of neoplastic or viral disease. Based on information as disclosed herein, a laboratory with capability in monoclonal antibody generation could produce a prototypic antibody that is predicted to interfere with activity of any transcription factor in the b-ZIP family. Further, a library of molecular clones capable of expressing a large number of antibodies could be screened to identify a clone with reactivity to the target region of any b-ZIP transcription factor. In either case, the sequence of the antibody could be obtained and structural data generated by X-ray crystallography or other procedures to proceed with the development of smaller molecules as described herein.

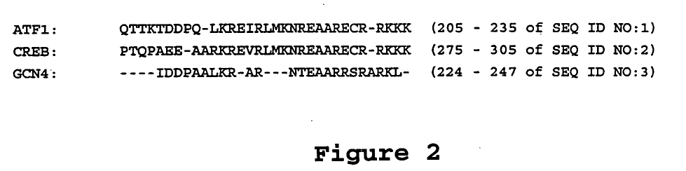

A model of sFv interaction with b-ZIP transcription factors on a CRE was discovered utilizing X-ray crystallography studies (Konig and Richmond, 1993). The structure of b-ZIP transcription factors is remarkably similar in the region depicted. A comparison of the sequences of ATF1, CREB and GCN4 is shown in FIG. 2. The locations of the subject regions are underlined. Significant variation in sequence in b-ZIP transcription factors does not begin until the subject region, outside the DNA binding domain but distinct from the activation domain. In FIG. 1, the predicted structure of sFv4 is shown adjacent one helix of ATF1 (containing the epitope of peptide c (residues 205-219 of SEQ ID NO:1)). Asparagine is located in the center of the major groove, as is typical in all b-ZIP transcription factors. The side chains of arginine residues are shown interacting with the phosphodiester backbone on both sides of the major groove. Interference with these stabilizing interactions by physical presence of the sFv may cause dissociation of either CREB or ATF1 to the CRE. Alternatively, sFv binds to the epitope of ATF1 or CREB when the transcription factor is free in solution and not bound to DNA. The interaction of the sFv with the key domain in a transcription factor then prevents the binding of the factor to DNA. Size relationships are relative to the 10 Å bar at the bottom right of the panel in FIG. 1.

Although the structure of b-ZIP proteins is, by definition, similar in the basic region that binds DNA, ATF1 structure is predicted to be significantly different from CREB, for example, in the region beyond the basic domain with considerably fewer prolines in this region and likely fewer random turns and complexity. Our preferred explanation for the ability of the sFv, but not the mAB4, to bind CREB relates to the probable complexity of CREB beyond the DNA binding region. CREB is predicted to have complex structure that does not allow for direct contact by the mAb4, but the sFv and other smaller compounds based on the structure and content of the CDR would be able to make contact with CREB and inhibit its function. In CREB, the first proline after the DNA binding domain is one turn more distant than in ATF1 and is followed by additional prolines which are predicted to result in more complex structure. The epitope of sFv in CREB is believed to be less accessible than that of ATF. mAb4, which has weak affinity for CREB, is 150,000 Daltons in mass and is unlikely to make a strong contact with the epitope adjacent the basic domain due to steric hindrance. The greatest dimension of an antibody across the divalent Fv portion measures 150 Å. It was discovered with computer modeling that the greatest dimension of the sFv is approximately 30 Å. However, a reduction in size as seen with the sFv (25 kD) would increase the likelihood for stronger interaction with CREB. The difference in Kd for mAb4 and sFv4 for ATF (1 nM vs. 3 nM) is not significantly different to see a change as measured by gel shift or studies in cells. Since the off rate of CREB and ATF1 is rapid, the presence of sFv in the region between the a helices may prevent rebinding of the factor to DNA.

Subcellular localization of sFv4. Nuclear localization signals (NLS) responsible for directing newly synthesized proteins to the nuclear pore complex are classically composed of short stretches of five to six basic amino acid residues such as the PKKKRKX sequence of SV40 large T antigen. The basic residues are thought to function by interacting with the ligand-binding domain of karyopherin α (inportin α) which mediates nuclear import. Existence of nonconventional NLS's which are discontinuous or multipartite, have been postulated for nuclear proteins including HTLV1 Tax, influenza NP, RSV MA and nucleoplasmin proteins, but a specific example has not been described and confirmed experimentally.

In order to investigate the subcellular localization of sFv4, it was fused to the green fluorescent protein (GFP). GFP was used as a fluorescent probe for monitoring the intracellular trafficking without disrupting the normal activity of its fusion partner. sFv4 was demonstrated to function through a nuclear mechanism as described in Examples 20 and 24.

Inhibition of oncogenic fusion protein EWS/ATF1. Of particular relevance is the fact that neoplasms (often of mesenchymal origin) may result from the translocation of chromosomes which results in the fusion of two (or more) proteins including an amino acid sequence that can be considered to be a linker domain of the invention. Application of the method of the present invention to oncogenic fusion proteins with transcription factor components was based in part on the knowledge that ATF1 is a component of the chimeric protein involved in the development of Clear Cell Sarcoma (CCS). The chimeric protein results from a chromosomal translocation where the ATF1 gene is fused with the gene associated with Ewings Sarcoma (EWS). The resulting EWS/ATF1 chimeric protein acts as a disregulated transcription factor. The availability of the anti-ATF1 sFv4 provided a means to explore the importance of DNA binding by fusion proteins such as EWS/ATF1 and to evaluate their role in the neoplastic process. Evidence presented herein demonstrates that the C-terminal region of EWS/ATF1 retains the mAb4 epitope and that this epitope is accessible for binding by sFv4. This chimeric fusion protein is believed to play a key role in the development of neoplasia where the activation domain of EWS protein is brought in close proximity to DNA by the action of the DNA binding domain of ATF1. Interference with the fusion protein activity through intracellular expression of sFv4 in a cell line derived from CCS, reduced CRE-driven reporter activity and viability and induced apoptosis. Demonstration of a prototypic approach to inactivate such an oncogenic fusion protein has application to other neoplasms resulting from chromosomal translocations. For example, human rhabdomyosarcoma is believed to arise from the oncogenic effect of a chimeric protein containing portions of forkhead and Pax proteins.

Evidence presented here regarding activity of sFv4 has been made even more important by new studies using a cell line from a human tumor in which a fusion protein containing ATF1 is over expressed (Bosilevac et al., in press). The chromosomal translaocation t(12:22)(q13:q12) associated with Clear Cell Sarcoma gives rise to a fusion protein in which the N-terminal 325 amino acids of the Ewings Sarcoma protein (EWS) replace the N-terminal 65 amino acids of ATF 1 (Bridge et al., 1990; Bridge et al., 1991). The tumor cell line is from a Clear Cell Sarcoma with a translocation of chromosomes 12 and 22 resulting in a chimeric fusion protein containing portions of the Ewings Sarcoma protein (EWS) and ATF1.

ATF1 is a member of the CREB/ATF subfamily of bZIP transcription factors that also includes CREB and CREM. These inducible transcription factors regulate transcription through binding as homodimers or heterodimers to cyclic AMP response elements (CRE) following activation of certain pathways such as protein kinase A (PKA). ATF1 is a weaker transactivator in vitro than CREB (Gilchrist et al., 1995; Orten et al., 1994). EWS/ATF1 is predicted to bind to CREs via the bZIP domain provided by the C-terminal region of ATF1, but it does not retain cAMP-inducible activation due to partial deletion of the kinase inducible domain located in the N-terminal 65 amino acids of ATF1(Li and Lee, 1998).

Brown et al. have shown in a heterologous cell type that EWS/ATF1 is a strong constitutive activator of some CRE containing promoters and a repressor of others(Brown et al., 1995). A plausible mechanism for transformation in Clear Cell Sarcoma involves the deregulated activation of CRE-containing promoters by the fusion protein. Other chimeric proteins, including the PAX/FKHR chimeric protein found in rhabdomyosarcoma, are capable of transforming cells in culture and EWS/ATF1 may function in a similar manner to initiate tumor cell proliferation(Paula et al., 1999). The development of cancer is believed to be a multi step process and downstream events may occur which render the tumor independent of the initiating event(Li and Lee, 1998). It is not known whether EWS/ATF1 or other chimeric proteins resulting from translocations are essential for maintenance of cell proliferation.

The intracellular expression of an sFv targeted against ATF1 inhibited DNA binding and transcriptional activation but did not result in loss of cell viability. In comparison, for example, the inhibition of chimeric fusion protein containing both ATF1 and the Ewing's sarcoma protein (EWS), induced apoptosis in the tumor cell type known as Clear Cell Sarcoma.

Malignant transformation is believed to be a multi-step process and chromosomal translocations that generate chimeric proteins such as EWS/ATF1 may initiate a cascade of events leading to cancer (Arevalo et al., 1993; Gao and Paul, 1995; and Glockshuber et al., 1992). The exquisite specificity of antibodies for defined targets presents numerous opportunities for disrupting protein-protein or protein-DNA interactions, particularly when the targeted structures are complex and not amenable to blockade by small molecules. Recently, scFvs have been used to achieve phenotypic knockout of cell surface or cytoplasmic target proteins involved in neoplasia such as Ki-ras, ErbB2, epidermal growth factor receptor and the IL2 receptor (Marasco, 1995; Duan et al., 1995; Graus-Porta et al., 1995; Griffiths et al., 1993). As an embodiment of the present invention, it was discovered that a similar approach could be used to disrupt activity of a nuclear protein and demonstrate its role in the neoplastic process. In SU-CCS-1 cells, interference with the activity of EWS/ATF1 could theoretically eliminate the initiating process leading to neoplasia and yet have no effect on tumor growth since other pathways may become dominant following transformation. Interference with DNA binding and transcriptional activity by the ATF1-inhibitory sFv demonstrated EWS/ATF1 is important for maintenance of tumor cell viability in addition to its previously proposed role in initiating the neoplastic process (Hileman et al., 1994). Although DNA binding was blocked, the EWS/ATF1 protein remained available for interactions with other proteins of the transcriptional apparatus (Churchill et al., 1994).

The predicted interactions between CRE DNA and ATF1 are based on the structural studies of GCN4 bound to CRE DNA by Richmond and Keonig (Grim, et al., 1996; and Hage and Twee, 1997). A conformational change in a linker domain of EWS/ATF1 may occur following binding by sFv4, or presence of the antibody may destabilize the important amino acid side chain interactions with the phosphate-DNA backbone. When EWS/ATF1 is not bound to DNA, the antibody may prevent binding of transcription factor to DNA by occupying a region adjacent to the DNA binding domain. Although the binding kinetics of EWS/ATF1 are not known, sFv4 has been shown to disrupt ATF1-DNA complexes, and the presence of sFv4 in the region between the a helices may also prevent rebinding of the factor to DNA. If immunodepletion is the mechanism, then the inhibitory effect of sFv4 on EWS/ATF1 may be due to the removal of transcription factor from the cellular pool by altering its intracellular processing or nuclear transport.

Fujimura (1996) has proposed that EWS is a negative regulator of ATF1 binding activity based on relatively lower intensity of recombinant protein complexes in gel shift assays and results from deletion mutant experiments (Fisher and Fivash, 1994). We also noted a significant difference in the relative binding affinity of recombinant EWS/ATF1 to the CRE as compared with recombinant ATF1 when measured by band intensity on EMSA. However, the intensity of EWS/ATF 1-CRE complexes using cellular extracts from either 293T or SU-CCS-1 cells was roughly equivalent to that seen with recombinant ATF1. Therefore post-translational modification of EWS/ATF1 may be important for regulating binding activity as has been shown for EWS/FLI (Hai et al., 1988). In direct comparison with ATF1, EWS/ATF1 greatly increases gene expression when measured by reporter assay (Fisher and Fivash, 1994; and Hileman et al., 1994). The increased expression with EWS/ATF1 is thought to result from either the loss of regulatory elements by truncation of ATF1 or the contribution of the potent EWS transcription activation domain (Chothia and Lesk, 1987). A quantitative comparison of EWS/ATF1 to other intracellular proteins in human tumors has not been previously demonstrated. Since the chimeric protein is not produced in the absence of the translocation between chromosomes 12 and 22, expression levels must be compared with other endogenous protein. As determined by cytogenetic analysis, a single allele of the wild type EWS and ATF1 genes remains intact in SU-CCS-1 cells. Our western blot experiments indicate that EWS/ATF1 is present in considerable excess to the endogenous levels of ATF1 in the SU-CCS-1 cell line and a CCS tumor. Densitometric analysis indicated that EWS/ATF1 is expressed at a 3.0 fold greater level than ATF1 in the SU-CCS-1 cell line and a 10.6 fold greater level in a CCS tumor. As originally suggested for Ewing's sarcoma, the EWS/ATF1 fusion protein may achieve transformation through both over-expression and strong transcriptional activation capability (Jameson and Sawyer, 1980). Similar explanations have been proposed for alveolar rhabdomyosarcoma associated with translocations of the PAX3 and FKHR protein genes (Kabat et al., 1992).

EWS/FLI, EWS/ATF1 and other chimeric proteins resulting from specific translocations in leukemias, lymphomas and sarcomas can be considered true tumor-specific proteins and the linker domain can serve as a unique epitope for derivation of antibodies. However, molecular modeling of the EWS/ATF1 chimeric protein suggested that the fusion junction was not an exposed surface and unlikely to be available for binding by antibody. As demonstrated with mAb5 (Example 9), binding of transcription factors by antibody does not necessarily result in loss of function in vitro. Intracellular expression of sFv4 reduced activity of the CRE containing proliferating cell nuclear antigen (PCNA) promoter by approximately 60%, but no loss of cell viability was seen when compared to controls (Darsley et al., 1985). HeLa cell transfections were performed and verified that sFv4 expression was not cytotoxic in cells without EWS/ATF1. No loss in viability was observed in transfected HeLa cells, which suggests that sFv4 induced cell death in SU-CCS-1 cells by disruption of EWS/ATF1 activity and not through inhibition of endogenous ATF1 activity.

The process of cell death in SU-CCS-1 cells exposed to sFv4 appears to have occurred through an apoptopic mechanism (Fisher et al., 1993). The finding that 30% of cells exposed to SRα-Fv4 were apoptotic as compared to controls (p<0.005) is comparable to results observed by others in studies of apoptosis (Koike et al., 1989; and Konig and Richmond, 1993). However, cell death involves multiple pathways and ultra-structural studies are helpful in determining whether evidence of necrosis is present (Gao and Paul, 1995).

Disruption of key molecular processes responsible for neoplastic transformation and reversal of malignant phenotypes are important goals in developing new cancer therapeutics (Kubota et al., 1996). The targeted disruption of EWS/ATF1 activity via the ATF1 epitope of sFv4 reduced SU-CCS-1 cell viability but had little effect on HeLa cells not expressing the oncogenic fusion protein. By demonstrating activity in this tumor cell type, we demonstrate the importance of chimeric proteins with transcriptional activity in maintenance of tumor cell viability. The evidence presented here has broad application to leukemias, lymphomas and other sarcomas with characteristic chromosomal translocations involving transcription factors such as the EWS/FLI-1 in Ewings Sarcoma and PAX3/FKHR in alveolar rhabdomyosarcoma. Because the level of the oncogenic EWS/ATF1 protein is higher in primary tumors than in established cell lines, and in vivo studies would be appropriate to determine the therapeutic potential for disruption of fusion protein transcriptional activity by antibodies.

Inhibition of oncogenic fusion protein EWS/FTL1. The use of intracellular sFv to induce apoptosis in CCS can be applied to other sarcomas with characteristic translocations involving DNA binding transcription factors, such as Ewing's sarcoma and primitive neuroectodermal tumor's (PNET).

Ewing's sarcoma and PNET are tumors of childhood and adolescence with a consistent chromosomal translocation (Busch et al., 1990; and Ellenberger et al., 1992). Ewing's Sarcoma and PNET are related if not the same tumor type and one observation supporting a common origin is the characteristic translocation involving the Ewing's sarcoma protein (EWS) and the Friend leukemia integration site 1 protein (FLI1) (May et al., 1993). The translocation results in the generation of a chimeric gene that joins the 5′ portion of the EWS locus to the 3′ region of the FLI1 gene resulting in the replacement of the transcription activation domain of FLI1 with EWS. This chromosomal translocation is found in over 90% of Ewing's sarcoma and PNETs, strongly suggesting the product of this rearrangement is critical for the development of these malignancies (Ladanyi, 1995). The reciprocal translocation does not result in an expressed protein due to the presence of an in-frame stop codon immediately C-terminal to the FLI1 sequence.

The ETS protein family includes a large family of related of transcription factors which bind DNA and appears to be involved in developmental processes and the cellular response to signaling pathways (Pio et al., (1996). The Friend leukemia integration site 1 protein (FLI1) is a member of the ETS family which also includes ETS1, ETS2, ERGB, ERG-1, SAP-1, PEA3, PU1 and ELK-1 which are involved in the activation of promoters containing a serum response element (SRE) (Magnaghi et al., 1996). ETS family members are helix-loop-helix proteins. All proteins in the ETS family share an 85 amino acid region referred to as the ETS domain which is commonly located at the c-terminus through which they specifically bind promoter elements displaying a consensus GGAA core sequences referred to as the ETS box. The nucleotides flanking the core sequence also contribute to the definition of sub-classes of ETS boxes. Evidence for the role of ETS family members in controlling gene expression were demonstrated by studies using the ETS box and DNA-binding for electromobility shift assays (EMSA). Related studies have shown that FLI1 binds only weakly to an SRE. However, in the presence of serum response factor (SRF), FLI1 forms a ternary complex with strong binding to the SRE (Magnaghi et al., 1996). Consistent with the activity of other members of the ETS family, FLI1 is a weak transforming protein. Both the DNA-binding activity and the transforming activity are greatly changed through its interaction with EWS as a fusion protein.

The EWS gene located on chromosome 22 is a surprisingly frequent participant in chromosomal translocations (Ladanyi, 1995). Different translocation partners of EWS include FLI1, ERG, ETV1, ATF1, CHOP and WT1. The cellular function of the EWS gene is presently unclear although one portion has been shown to demonstrate RNA-binding activity (Speleman et al., 1990). RNA-binding proteins are typically involved in post-translational regulation of gene expression but in the context of other DNA-binding proteins, the EWS protein appears to significantly alter gene expression. Bertolotti, et al, suggested EWS may also function as a transcription factor due to a high degree of homology with the TBP-associated factor hTAF_{II}6 (Bertoloitti et al., 1998). EWS was shown in studies by Pan, et al, to possess multiple determinants that cooperate synergistically to activate transcription, but by itself, EWS was not capable of binding to DNA (Pan et al., 1998)). EWS is ubiquitously expressed and is a nuclear protein. Also important to the underlying pathogenic mechanism is the retention of the EWS promoter in the chimeric gene, driving expression of the fusion protein. The EWS promoter is constitutively active and is presumably responsible for the high level expression of EWS/FLI and other fusion proteins in which it is a component.

Although FLI1 and EWS/FLI1 have been shown to bind to DNA through the identical c-terminal portion of the proteins, EWS/FLI1 recognizes target sequences distinct from those bound by wild type FLI1 (Magnaghi et al., 1996). Thus EWS/FLI1 and FLI show not only quantitative differences in transactivation ability but also differences in binding activity. In comparisons of the transcriptional activity of FLI1 by itself and in combination with EWS/FLI1, the latter has much higher transcriptional activity on homologous promoters. Further, although the wild type FLI1 is weakly transforming, the EWS/FLI fusion protein has higher transforming activity in fibroblasts and induces expression of other proteins implicated in the neoplastic process (May et al., 1993).

An anti-FLI1 single chain variable fragment (sFv) can be developed, using the evidence presented herein, to investigate whether EWS/FLI is necessary for induction of neoplasia and also maintenance of the malignant phenotype and to determine whether disruption of DNA-binding by FLI1 in the context of the fusion EWS/FLI1 will induce apoptosis in Ewing's Sarcoma and PNET cells. An sFv can be developed which targets a region immediately outside of the DNA-binding region of FLI1, the linker domain, such as shown in the studies with ATF1 to be a key target for inhibition of DNA-binding.

Inhibition of oncogenic fusion protein PAX/FKHR. The model of tumor cell killing through disruption of DNA-binding by intracellular sFv can be expanded to sarcomas, for example, Rhabdomyo sarcoma (ARMS). The oncogenic origins of Rhabdomyosarcoma are believed to be related to a characteristic chromosomal translocation t(2:13) (q35:q14) (Ladanyi, 1995). This typical cytogenetic finding is considered diagnostic of ARMS, although other translocations have been described. The t(2:13) translocation involves the PAX 3 and forkhead (FKHR) genes and results in the fusion protein PAX3/FKHR The less common translocation t(1:13) involves the PAX 7 gene and FKHR genes. The specific association between the t(2:13) translocation and ARM strongly suggest that the resulting chimeric protein plays a primary role in the development of the tumor. The mechanism of oncogenesis is believed to occur through increased transcriptional activation of the fusion PAX 3/FKHR protein in comparison with the wild-type PAX 3, but a number of related effects may combine to achieve transformation (Fredericks et al., 1995).

Recent studies have identified the PAX DNA binding motifs as responsible for the transforming capability of PAX/FKHR (Lam et al., 1999). Homeodomain and paired box proteins have been shown to bind to DNA with their core sequences. The structural features of one member of the PAX family bound to DNA have been studied by x-ray crystallography (Wilson et al., 1995). These studies have revealed the third helix lies deep within the major groove and has specific contacts with nucleotides in both strands of the core sequence. The DNA-binding domain is similar to helix DNA-binding proteins and contacts a ten base pair region of duplex DNA. Several features are similar to that of CRE-binding proteins such as CREB and ATF1 in that while the key recognition helix interacts with the central core, additional important contacts are made with the phosphate backbone of either side of the core sequence. In addition, a proline is predicted to terminate the alpha helix structure which resembles the epitope of sFv4 described previously (Orten et al., 1994).

The human paired box (PAX) genes compose a family of transcription factors that play a fundamental role in the regulation of development such as the kidneys and genital tracts (PAX 2) B cells (PAX 5), eye structures (PAX 6) and muscle development (PAX3 and PAX 7) (Hinrichs et al., 1984). Following muscle cell differentiation both PAX 7 and 3 are down-regulated. PAX 3 is also implicated in the migration of muscle cell precursors suggesting a critical role in myogenesis. The forkhead family of transcription factors includes FKHR however the specific contribution of FKHR to oncogenesis is uncertain. Recently, deletion studies of PAX/FKHR have shown that mutations of the FKHR activation domain are unable to transform NIH 3T3 cells (Lam et al., 1999). Therefore, FKHR is thought to contribute to oncogenesis through its effect on protein-protein interactions of factors involved in transcription. PAX proteins and other proteins involved in cell differentiation and normal development are expressed at specific time points in cell development and are subsequently down-regulated in conjunction with differentiation. Therefore, interference with their endogenous activity in fully differentiated cells may not have untoward biological effect.

Genetic research has identified many different targets for development of anti-cancer therapeutics. An anti-sense oligonucleotide strategy has been used to specifically down-regulate expression of the PAX 3/FKHR fusion protein (Bernasconi et al., 1996). The introduction of anti-sense oligonucleotides into rhabdomyosarcoma cells in culture resulted in the induction of apoptosis. In addition to supporting the hypothesis that PAX 3/FKHR plays a role in tumor development, these studies also suggested that PAX 3/FKHR may be essential for cell survival. A number of oncogenes and tumor suppressor genes have been implicated in the process of apoptosis including bcl-2, the retinoblastoma and the Wilms' tumor proteins (Raffray and Cohen, 1997). Although several explanations exist for the potential mechanism, one possibility is that these proteins influence protein interactions involved in the control of cell cycle. Others have proposed that PAX and PAX FKHR genes influence expression of other proteins involved in apoptosis since the DNA binding domain of PAX is retained in the fusion transcript. Although oligonucleotide therapy has had little success in vivo, the important studies of Bernasconi clearly demonstrate that the inactivation of PAX protein should be explored as a treatment for Rhabdomyosarcoma (Bernasconi et al., 1996).

Method of Use: Rationale Drug Design.

The goal of rational drug design is to produce structural analogs of biologically active molecules of interest or of small molecules with which they interact (e.g., agonists, antagonists, inhibitors) in order to fashion drugs which are, for example, more active or stable forms of the biologically active molecules, or which, e.g., enhance or interfere with the function of a biologically active molecule in vivo. See, e.g., Hodgson, 1991. In one approach, one first determines the three-dimensional structure of a molecule of interest (e.g., a transcription factor) or, for example, of the transcription factor-ligand complex, by x-ray crystallography, by computer modeling or most typically, by a combination of approaches. Less often, useful information regarding the structure of a biologically active molecule may be gained by modeling based on the structure of homologous biologically active molecules. An example of rational drug design is the development of HIV protease inhibitors (Erickson et al., 1990). In addition, peptides are analyzed by an alanine scan (Wells, 1991). In this technique, an amino acid residue is replaced by Ala, and its effect on the peptide's activity is determined. Each of the amino acid residues of the peptide is analyzed in this manner to determine the important regions of the peptide.

It is also possible to isolate a target-specific antibody, selected by a functional assay, and then to solve its crystal structure. In principle, this approach yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides or other molecules from banks of chemically or biologically produced banks of peptides and other molecules. Selected molecules would then act as the pharmacore. Thus, one may design drugs which have, e.g., improved activity or stability or which act as inhibitors, agonists, antagonists, etc. of transcription factor activity.

Following identification of a substance which modulates or affects polypeptide activity, the substance may be investigated further. Furthermore, it may be manufactured and/or used in preparation, i.e., manufacture or formulation, or a composition such as a medicament, pharmaceutical composition or drug. These may be administered to individuals.

Thus, the present invention extends in various aspects not only to a substance identified using a funtional domain of a transcripiton factor identified herein as a modulator of transcription factor activity, in accordance with what is disclosed herein, but also a pharmaceutical composition, medicament, drug or other composition comprising such a substance, a method comprising administration of such a composition comprising such a substance, a method comprising administration of such a composition to a patient, e.g., for treatment of cancer, use of such a substance in the manufacture of a composition for administration, e.g., for treatment of cancer, and a method of making a pharmaceutical composition comprising admixing such a substance with a pharmaceutically acceptable excipient, vehicle or carrier, and optionally other ingredients.

A substance identified as a modulator of transcription factor function may be peptide or non-peptide in nature. Non-peptide small molecules are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing is generally used to avoid randomly screening large numbers of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide, e.g., by substituting each residue in turn. Alanine scans of peptide are commonly used to refine such peptide motifs. These parts or residues constituting the active region of the compound are known as its “pharmacophore”.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted onto it can conveniently be selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent they exhibit it. Further optimization or modification can then be carried out to arrive at one or more final mimetics for in vivo or clinical testing.

A preferred therapeutic composition of the present invention is either a short glycopeptide, reminiscent of Tacrolimus (FK508) or a carbon based drug derived by rational design using structural information according to the present invention. Alternatively, a diabody approach could be used to deliver the sFv to a selected cell type or neoplastic cell. A diabody consists of two separate sFv's that are allowed to dimerize or are designed to dimerize, with each component having different specificity (Whitlow et al., 1993; Luo, 1995). A likely target would be a cell surface receptor (such as EGFR) that is over expressed in the tumor cell of interest. Binding of receptor is followed by internalization of the partner sFv with anti-transcription factor activity. The presence of such cell surface targets in the CCS cell line could be identified and feasibility studies could be carried out in culture and then in the mouse tumor model.

Another alternative for cancer therapy would be to combine the characteristics of the specific antibody, such as sFv4creb or sFv4atf, with those of catalytic antibodies described by Dr. S. Paul (Univ. Ne. Med. Cntr.). The catalytic antibody could combine, for example, the heavy chain of the ATF or CREB specific sFv with a catalytic light chain selected for activity against the sequence adjacent to the binding domain of the VH. Cleavage of the transcription factor at this site would be expected to generate a negative regulating competitor of the transcription factor that could not respond to activation due to loss of activation domain.

According to the methods of the present invention, tissue specific transcription factors with an identified linker domain are targeted and used for generation of a new sFv. New transcription factors are being described on a regular basis and in some cases these transcription factors have greater tissue specificity than ATF 1 and CREB and play unique roles in regulating defined processes such as the shift from TH1 to TH2 lymphocytes.

Pharmaceutical Compositions and Routes of Administration

The modulators identified in accordance with the present invention can be formulated in pharmaceutical compositions, which are prepared according to conventional pharmaceutical compounding techniques. See, for example, Remington's Pharmaceutical Sciences, 18th Ed. (1990, Mack Publishing Co., Easton, Pa.). The composition may contain the active agent or pharmaceutically acceptable salts of the active agent. These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the form of preparation desired for administration, e.g., intravenous, oral, intrathecal, epineural or parenteral.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

The active agent is preferably administered in a therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remingtons Pharmaceutical Sciences.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

Definitions

The present invention employs the following definitions.

“ATF1” refers to activating transcription factor 1.

“Activation domain” and “Transcription activation domain” or “TAD” refer to a functional domain which interacts with other proteins and influences transcription initiation.

“b-ZIP” refers to basic leucine zipper transcription factor.

“CCS” refers to Clear Cell Sarcoma.

“CRE” refers to cyclic AMP response element.

“CREB” refers to cyclic AMP response element binding protein.

“CREM” refers to cyclic AMP response element modulator.

“Characteristic chromosome translocation” refers to a genetic feature common to a particular phenotype that results from the exchange or movement of a portion of a chromosome to a different chromosome or location.

“EMSA” refers to electrophoretic mobility shift assay.

“EWS” refers to Ewings Sarcoma Protein.

“Epitope and/or antigenic epitope” refers to that portion of a molecule to which specific binding by an antibody (or derivative) occurs.

“FKHR” refers to forkhead transcription factor.

“FLI” refers to friend leukemia virus insertion.

“Inhibitory agent” refers to an antibody; subcomponent of an antibody, such as Fab fragment, sFv subunit, or diabody; a polypeptide representing the configuration of the antibody binding site (peptide mimetic) and possessing the essential binding features of the antibody; or small molecules that resembles the configuration of the antibody binding site and possesses the essential binding features of the antibody, such as glycopeptide (non-peptide mimetic): provided that in each case the inhibitory agent is capable of binding specifically to the intended linker domain on the transcription factor with consequent prevention or inhibition of transcription.

“Linker domain” refers to the connecting region, with or without independent functional activity, lying between an effective DNA binding domain and an activation domain of a transcription factor, including without limitation, oncogenic fusion proteins.

“mAb” refers to monoclonal antibody.

“Mimetic” refers to a substance which has the essential biological activity of the sFv. A mimetic may be a peptide-containing molecule that mimics elements of protein secondary structure (Johnson et al., 1993). The underlying rationale behind the use of mimetics is that the peptide backbone of proteins exists chiefly to orient amino acid side chains in such a way as to facilitate molecular interactions, such as those of antibody and antigen, enzyme and substrate or scaffolding proteins. A mimetic is designed to permit molecular interactions similar to the natural molecule. A mimetic may not be a peptide at all, but it will retain the essential biological activity of natural sFv.

“Oncogenic fusion protein” refers to an oncogenic protein which acts as a disregulated transcription factor, and which results from a chromosomal translocation.

“PAX” refers to paired box transcription factor.

“PCNA” refers to proliferating cell nuclear antigen.

“sFv” refers to short chain variable antibody fragment. sfv is also sometimes referred to as scFv.

“Tumor specific fusion protein” refers to an oncogenic protein which acts as a disregulated transcription factor and which results from a chromosomal translocation that is found to be unique or limited to a narrow range of tumor types.

The practice of the present invention employs, unless otherwise indicated, conventional techniques of chemistry, molecular biology, microbiology, recombinant DNA, genetics, and immunology (Maniatis et al., 1982; Sambrook et al., 1989; Ausubel et al., 1992; Glover, 1985; Anand, 1992; Guthrie and Fink, 1991).

### EXAMPLES

The present invention is described by reference to the following Examples, which are offered by way of illustration and are not intended to limit the invention in any manner. Standard techniques well known in the art or the techniques specifically described below are utilized.

### Example 1

### Methods and Material

The following preparations and methodologies are those utilized in the Examples, unless otherwise indicated.

Preparation of Recombinant CREB. Recombinant CREB was produced using CREB coding sequences, prepared according to Zhao and Giam (1992). The cDNA for CREB was cloned according to the methodology of Studier et al. (1990), at the NdeI/BamHI sites of the pET-11a expression plasmid. The protein was expressed from the bacteriophage T7 promoter and was purified from Escherichia coli cell lysates on DNA-cellulose columns (Sigma).

Preparation of Recombinant ATF1. Recombinant ATF1 was produced using expression vectors containing full length ATF1, according to L. J. Zhao and C. Z. Giam (1992). The cDNA for ATF1 was cloned according to the methodology of Studier et al. (1990), at the NcoI/BamHI sites of pET lid. The protein was expressed from the bacteriophage T7 promoter and was purified from Escherichia-coli cell lysates on DNAcellulose columns (Sigma).

Preparation of Nuclear Extracts. Nuclear extracts were prepared from 1-5×10⁸ cells as described by Dignam et al. (1983), and dialyzed against 20 mM HEPES (N-[2-Hydroxyethyl]piperazine-N′-[2-ethanesulfonic acid]), pH 7.9, 100 mM KCl, 2 mM dithiothreitol, 20% glycerol, 0.2 mM EDTA (ethylenediamine tetraacetic acid), 1 mM PMSF (phenylmethylsulfonyl fluoride), 20 μg/ml aprotinin and 10 μg/ml trypsin-chymotrypsin inhibitor. Alkaline phosphatase treated nuclear proteins were prepared by digesting nuclear extracts (150 μg protein/reaction) with 20 units of calf intestine alkaline phosphatase (New England Biolabs, 1993) in 50 mM Tris, pH 9.5, 50 mM NaCl, 5 mM MgCl₂ at 37° C. for 1 hr. Total protein was determined by the Bradford Assay (Biorad, 1993) and amounts of ATF1 and CREB were estimated by Western blot analysis, as hereinafter described.

Preparation of monoclonal antibodies. ATF1 monoclonal antibodies were generated using 3 10 μg injections of recombinant ATF1, prepared as described above, as immunogen with the Ribi Adjuvant System (Ribi Immunochem Research Inc.; Masihi, 1989). The method used for generating the monoclonal antibodies was that of Kohler and Milstein (1976). The panel of MAbs were screened initially by ELISA (Volker and Bidwell, 1986) on plate-bound recombinant ATF1. Isotypes were determined using a kit from Amersham. All MAb had κ light chains, MAb1, 3 and 4 were IgG1 isotype and MAb5 was an IgA isotype. Antibody affinity was evaluated by competitive ELISA (Friguet et al., 1985) using recombinant ATF1 as an antigen.

IgG1 MAbs used in DNA binding and in vitro transcription assays were affinity purified on a protein G column and quantitated by spectroscopy at A₂₈₀ and the Bradford Assay (Biorad protein assay). IgA antibodies in ascites fluid were quantitated by scanning IgA light chain on dried Coomassie blue stained SDS-PAGE gels with a ScanMaker 600ZS (Microtek, Inc.) and analyzed using the “Image” program on a Macintosh IIci computer. This analysis determined that the MAbs IgA concentration was 10 mg/ml whereas the control was 15 mg/ml.

Anti-CREB antibody used for western blot analysis and DNA binding assays was a rabbit polyclonal antibody against the CREB α-peptide (Santa Cruz Biotechnology). Isotype matched myeloma proteins IgG1, κ(MOPC) (SIGMA), and IgA, κ(TEPC) (Chothia et al., 1989) were used as negative controls for the MAb assays.

Western Blot Analysis. Proteins were resolved by SDS PAGE electrophoresis on 15% polyacrylamide gels and transferred to nitrocellulose. Nonspecific binding was blocked with 10% powdered milk in Tris buffered saline plus 0.1% Tween 20 and membranes were incubated for 1 hour with hybridoma tissue culture supernatants. Supernatant from each of the monoclonal cultures 1, 3, 4, and 5 were used separately and prepared in accordance with the methodologies set forth above, diluted 1:4 in Tris buffered saline. Bound antibody was detected with a commercial biotin-streptavidin-enhanced detection kit (Amersham, 1993) used according to the manufacturer's instructions.

Preparation of mAbs4 and sFv4. Preparations of mAb4 were affinity purified on a protein G column and quantitated by absorbance at 280 nm with the Bradford Assay. Soluble sFv4 was produced and quantitated as described by Ohno et al. (1994). E. coli HB21, incubated until reaching an A₆₀₀ of 0.6, were induced with isopropyl-D-thiogalactopyranoside (IPTG) and incubated an additional 4 hours at 25° C. The periplasm was extracted in a high-salt lysate buffer, clarified and dialyzed. sFv4 quantitation was performed through slot blotting of the periplasmic extract and a peptide standard. The slot blots were stained with an anti c-myc-tag Ab (murine 9E10 hybridoma, ATCC) and an alkaline phosphatase (AP)-conjugated anti-mouse IgG heavy and light (H&L) chain Ab (Jackson ImmunoResearch Laboratories, West Grove, PN). A standard curve (1-100 ng) using c-myc-peptide-1 (Oncogene Research Products, Cambridge, Mass.) was generated and the signal of sFv wells was visually compared for determination of approximate concentration and digitally scanned for densiometric analysis. Following normalization for mass (mass of c-myc peptide=mass of sFv/8) the average periplasmic concentration of sFv was observed to be 5 ng/ml.

RT-PCR and Isolation of EWS/ATF1 cDNA. Total RNA mini-preps were prepared following manufacturer's directions from 100 mm dishes of SU-CCS-1 cells using Quiagen RNeasy and QuiaShredder columns (Quiagen, Valencia, Calif.). 50 ng of total RNA was reverse-primed with an oligo poly-dT primer and extended with Superscript™ reverse transcriptase (Gibco, Lifetech, Grand Island, N.Y.) according to established protocols. The EWS/ATF1 fusion was amplified from the product of the cDNA synthesis by PCR with appropriately designed primers based on the genebank ATF1 and EWS sequences. A PCR product of approximately 1600 bp was obtained and ligated into the T/A cloning vector (Invitrogen, Carlsbad, Calif.) for screening and sequencing. Multiple colonies were screened using mini-prep spin columns (Quiagen), and those containing the properly sized insert were submitted for automated sequencing.

DNA constructs. For intracellular expression assays, the cDNA of EWS/ATF1 was cloned into pCMV4 (Darsley et al., 1985). The EcoRI-HindIII fragment from T/A-EWS/ATF1 was inserted into the BglII-HindIII sites of pCMV4 to generate the vector referred to as pEWS/ATF1 and used to generate protein in 293T cells. The vectors pATF1 and pFv4 are as previously described (Darsley et al., 1985). The EWS/ATF1 cDNA was inserted into the EcoRI site of pET29(b) (Novagen, Madison, Wis.) which had the NcoI-EcoRV fragment removed. This construct, pET-EWS/ATF1, was screened for orientation and used for the in vitro generation of recombinant protein in E. coli BL21.

Preparation of recombinant proteins. Recombinant EWS/ATF1 was generated by in vitro transcription-translation (iTT) using the TnT® T7 Quick Coupled Transcription/Translation System (Promega, Madison, Wis.) according to manufacturer's instructions. Both ³⁵S labeled and unlabeled recombinant proteins were generated for use as markers in western blot and EMSA. Recombinant EWS/ATF1 and ATF1 were also generated through IPTG induction of ATF1 cDNA and EWS/ATF1 cDNA containing pET vectors in E. coli. BL21 (Zhao and Giam, 1992). ATF1 expressing bacteria were boiled for 20 minutes as described by Zhao and Giam (1992). EWS/ATF1 was isolated as the insoluble protein fraction of induced bacteria according to established protocols (Marasco, 1995). Additionally, EWS/ATF1 was generated in 293T cells following transfection with pEWS/ATF1 and isolation of the nuclear extract using established protocols.

Electrophoretic Mobility Shift Assays. Electrophoretic mobility shift assays (EMSA) were performed (Orten et al., 1994; Gilchrist et al., 1995). Incubations were conducted at 30° C. after determining that EWS/ATF1 forms more intense complexes with the CRE at this temperature. ³²P-labeled oligonucleotide containing the consensus CRE: 5′-AGA GAT TGC CTG ACG TCA GAG AGC TAG-3′ was incubated with 50 ng of full length recombinant ATF-1 from E. coli BL21 or EWS/ATF1 from 293T cells. The binding reactions were done in the presence or absence of mAb4, mAb5, EWS-N and species and isotype matched controls. Following electrophoresis, the bound and unbound fractions of labeled oligonucleotide were quantitated by autoradiography for 12 hours using a PhosphorImager (Molecular Dynamics). The PhosphorImager data were exported as TIFF files and used to prepare FIGS. 1B and 1C.

Immuno-Blot Assays. Protein extractions from HFF and SU-CCS-1 cell lines were made using triple detergent saline (TDS) lysis buffer (1.0% Triton X-100, 0.5% deoxycholate and 0.1% lauryl sulfate (SDS)). Protein extraction efficiencies were determined by examining the relative amount of EWS/ATF1 and/or ATF1 in the insoluble cell membrane fraction as compared to the TDS soluble fraction. The insoluble fraction remaining from the original TDS extraction was re-solublized in 1% SDS and DNA was sheared by sonication. The samples were boiled for 10 minutes and analyzed by SDS-PAGE. Immuno-(Western) blots were performed as described by (Cho, et al., (1994)). Protein extraction from a clear cell sarcoma tumor was performed by mechanical homogenization in the presence of TDS lysis buffer. Protein concentrations were determined for each extract using the Bradford Assay Kit (BioRad). Immunoprecipitation was performed using mAb1 and mAb5 concurrently and 20 μL of Protein A Sepharose (6 μg/μL) incubated with 150 ng of cellular or tumor extract for 150 minutes at 4° C. Efficiency of immunoprecipitation was determined by comparison of pre- and post-immunoprecipition and supernatant fractions by SDS-PAGE and transfer to nitrocellulose. Membranes were incubated with either 1 μg/mL mAb5 followed by an alkaline phosphatase (AP) conjugated goat-anti-mouse heavy and light (H&L) chain secondary antibody (Jackson ImmunoResearch) or EWS-N (SantaCruz BioTech) followed by an AP-conjugated mouse-anti-goat antibody (SantaCruz BioTech). The stained western blots were digitally scanned using a UMAX Astra 610s scanner to generate transfer image file format (TIFF) images that were imported into Canvas version 5.0.3 and used to prepare FIG. 1D. in vitro S³⁵ labeled EWS/ATF1 analyzed by autoradiography migrated identically to the presumed EWS/ATF1 band generated by western blot, thus confirming the identity of the EWS/ATF1 band. Analysis of band intensity was performed on the stained blots using a densitometer (Molecular Dynamics).

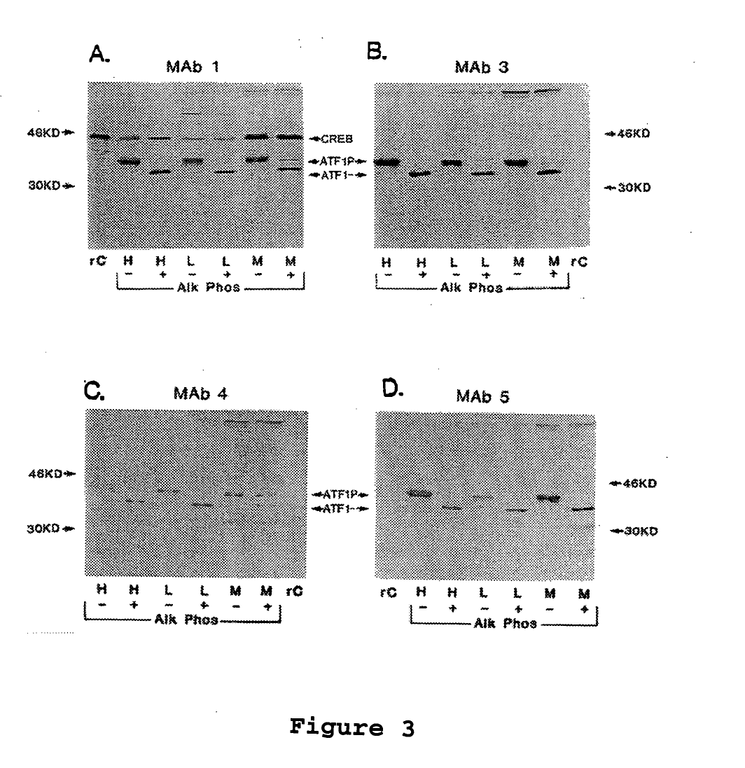

Transient Cotransfections and Luciferase/β-Galactosidase Assays. Transient cotransfections of HeLa cells were performed according to established protocols using calcium phosphate precipitation (Darsley et al., 1985). The transfections were performed in duplicate 35 mm wells containing 5 μg of the CMV-luc (CRE-luc) reporter construct and a RSV-β-galactosidase construct (2 μg) to control for variations in transfection efficiency. Cotransfections included increasing amounts of the EWS/ATF1 vector at 0, 5, 10 and 20 μg and the presence of plasmids pFv4 and pATF1. Additionally, a molar equivalent of parent vector (without cDNA insert) was used to maintain an equal number of promoter units in each transfection. The cells were harvested at 48 hours post-transfection, and the reporters were assayed. Transient cotransfections of SU-CCS-1 cells were performed using a similar approach of increasing amounts of pFv4. To facilitate efficient transfection of SU-CCS-1 cells, liposome mediated transfection was used with the Lipofectamine PLUS system (GIBCO/LifeTech) and cells were harvested at 72 hours. Measurement of reporter activity of firefly luciferase was determined as described relative to an internal β-galactosidase standard. Following transfection, cell extracts were prepared by freeze-thaw lysis in a potassium phosphate buffer. ATP and luciferin were added, and light emission was measured with a Luminoskan RS (Lab Systems/Denley, Franklin, M A) microplate luminometer. β-galactosidase expression was quantitated through the addition of o-nitrophenyl-β-d-galactopyranoside (ONPG) and the absorbance at 405 nm was measured on an ELISA plate reader. The luciferase value of each well was normalized to the internal β-galactosidase reporter. Results of three to five experiments were then averaged to generate the data depicted in FIGS. 2 and 3.

Production of Retrovirus and Infection of cells. Retroviral vectors were produced by inserting the EcoRI-HindIII fragment of pFv4 which contains the cDNA of sFv4 into the SRα-PN retrovirus (Takabe et al., 1988; and Kirschmeier et al., 1988) at the corresponding sites and the pCMV5 polylinker inserted into the HindIII site. To infect SU-CCS-1 cells, the SRα-Fv DNA construct was cotransfected into 293T cells with the amphotrophic packaging vector, psi(−) ampho. 10 μg of each was transfected using the Lipofectamine system described above. The cellular supernatant was collected every 12 hours between 24 and 72 hours post infection and pooled. The retroviral titer was determined by colony forming assay in 3Y1 cells grown in MEM containing 5% bovine calf serum (BCS) and 800 mM G418 (Geneticin). Typical yields of retrovirus were 10⁴ cfu/mL. Infection of cells was performed using 3 mL of retroviral stock/well in a 6 well plate in the presence of 4 mg/mL hexadimethrine bromide (polybrene). Plates were spun at 1250×g in a refrigerated centrifuge at 18° C.

Cell Viability Determinations—trypan blue exclusion and MTS assays. The viability of SU-CCS-1 cells infected by SRα-Fv4 or control SRα-PN was determined by trypan blue stain exclusion. Cells were harvested from 35 mm dishes with a rubber policeman, suspended in MEM and transferred to centrifuge tubes. The cells were washed in PBS and resuspended in 1 mL PBS. An equal amount of cell suspension was added to 2× trypan blue stain and the cells were counted in a hemocytometer. Grids were counted to quantitate blue cells and white cells until a minimum of 400 was obtained. In order to avoid the mechanical harvesting which could interfere with viability measurements, an MTS assay was performed using the CellTiter 96 Aqueous non-radioactive proliferation assay (Promega) which is a colorimetric method for determining the number of viable cells in proliferation assays. The assay is composed of the tetrazolium compound 3-(4,5-dimethylthiazol-2-yl)-5-(3-carboxymethoxyphenyl)-2-(4-sulfophenyl)-2H-tetrazolium (MTS) and the electron coupling reagent phenazine methosulfate (PMS). MTS is bioreduced by cells into a formazan which is soluble in tissue culture medium. The absorbance of the formazan at 490 nm can be measured directly from 96-well assay plates without additional processing. The conversion of MTS into the aqueous soluble formazan is accomplished by dehydrogenase enzymes found in metabolically active cells. Therefore, the quantity of formazan is directly proportional to the number of living cells in culture. For this assay, SU-CCS-1 cells were plated at 2×10⁴ cells/well and infected with SRα-Fv4 or controls (0.2 ml/well). Cell infections were conducted over 7 days to generate a time course of viability. On day 7, 96-well plates were incubated with the MTS assay reagents, and the absorbance was measured. The results of 3 to 6 experiments were normalized and plotted as percent viable cells versus time. The same MTS procedure was used to study the effect of SRα-Fv4 and control treatments on HeLa cell viability over a 4 day time course.

Apoptosis Measurements—flow cytometery and TUNEL staining. 50 μL of the washed cell suspensions from the tryptan blue exclusion determinations were plated on glass slides, air dried and fixed in 50% acetone/50% methanol. The remaining cell suspension was pelleted and fixed in 70% ethanol. The ethanol-fixed cells were prepared for DNA content analysis and apoptosis measurement by flow cytometery by washing in PBS and staining with propydium iodide (Telford reagent) overnight (Fine et al., 1986). Measurements were made using a Becton Dickinson FACStar^{PLUS} flow cytometer, and the data-set was analyzed using ModFit DNA modeling software (Versity Software, Topsham Me.). The slides of fixed cells were stained for apoptosis by in situ labeling of DNA breaks using terminal deoxynucleotide transferase (TdT) mediated dUTP-biotin nick end labeling (TUNEL) (Fisher et al., 1993). TdT was used to incorporate biotinylated deoxyuridine at sites of DNA breaks, and the signal was amplified by avidin-peroxidase and photographed under light microscopy.

Immunization of mice and generation of cDNA. Several alternatives exist for the generation of an sFv including the screening of a previously generated heavy and light chain cDNA library, immunization of mice and generation of heavy and light chain cDNA, or generation of monoclonal antibodies followed by cloning of the sFv (Churchill et al., 1994; and Darsley et al., 1985). Because the prior immunization of mice has been shown to increase the number of clones represented in the library by 100 fold, therefore decreasing the number of clones needed to be screened, this method is preferred for the generation of cDNA.

Exemplary of this approach is the generation of cDNA for PAX. Mice were immunized three times, three weeks apart, with 50 μg of both synthetic PAX peptide adjacent to the third alpha helix of the homeodomain and truncated recombinant PAX3, according to an IUCUC protocol (Univ. of Nebr. Medical Center). Administration is in RIBI adjuvant. The third helix of PAX, from α 260 to 276, mediates DNA contact as shown by x-ray crystallography (de la Paz et al., 1986). Fausman-Chou analysis was performed to identify a region with high antigenic index which incorporates a proline predicted to terminate the helix. Selection of a target based on these parameters results in an antibody capable of blocking DNA binding (Chotia and Lesk, 1987). Recombinant PAX is also used to confirm recovery of a clone targeting the region of interest. Five days after the second dose, the mice are bled and serum collected for detection of antibodies to PAX. The final immunization is with recombinant PAX/FKHR. If no reactivity is detected after the first two immunizations, dosage can be increased, for example to 100 μg. Five days after the final dose, the mice are sacrificed and the spleens removed for extraction and purification of RNA. Total RNA is purified using standard protocol and cDNA is generated using a kit (Invitrogen). The cDNA is then utilized as described in Example 22.

Screening of anti-sera and sFv clones by competitive ELISA. The reactivity of serum from immunized mice is evaluated by ELISA using recombinant PAX proteins. Following the cloning of sFv's, competitive ELISA using recombinant PAX and/or PAX/FKHR coated on microtitre wells as previously described is used to identify PAX specific sFv clones with the greatest relative affinity for further evaluation in gel shift assay. Increasing concentrations of protein are introduced into the solution containing sFv over a range from 0.01 μM to 1 μM and added to microtitre wells with antigen fixed to the plastic. Detection of bound sFv is accomplished with the polyclonal goat anti-mouse Fab antibody and a peroxidase conjugated donkey anti-goat antibody. After addition of substrate the plate is read and results are plotted as percent inhibition of wells. Following the mapping of the epitope, competitive ELISA is again performed to confirm affinity using peptide epitope as competitor.

### Example 2

### Characterization of ATF1 MAbs

The following example demonstrates that MAb1, 3, 4, and 5 react with untreated or alkaline phosphatase treated ATF1 on western immunoblots of nuclear extracts from human and murine cell lines.

Immunoblotting. The MAb were tested as reagents for immunoblotting. Nuclear extracts (15 μg per lane) from HeLa human cervical epithelioid carcinoma cells (H), L929 murine connective tissue fibroblasts (L), or MT-4 HTLV-1 transformed human T cells (M) were analyzed on 15% SDS-PAGE gels with (+) or without (−) calf intestine alkaline phosphatase (Alk Phos) treatment. rC indicates purified recombinant CREB protein (15 ng per lane).

Results indicate that all 4 MAb react with untreated or alkaline phosphatase treated ATF1 on western immunoblots of nuclear extracts from human and murine cell lines (FIG. 3). ATF1 also was readily detected in whole cell extracts from established cell lines. Only MAb1 reacted with phosphorylated and dephosphorylated CREB in nuclear extracts. MAb1, 3 and 5 detected as little as 0.5-1 ng of recombinant ATF1 on immunoblots; however 5-10 ng was required for reaction with MAb4.

Transcription Factor Detection Assay (TFDA). Whether an antibody or compound constitutes an inhibitory agent of this invention can be determined by testing the antibody or compound in the TFDA. This assay evaluates the candidate agent for its ability to inhibit ATF1 binding to DNA in the electrophoretic mobility shift assay herein referred to as the TFDA. The TFDA is generally simpler, faster, and more sensitive than other methods for detecting sequence-specific DNA-protein binding. Separate lanes of the gel are used for the following compounds respectively: 1) DNA alone; 2) DNA with ATF1; 3) DNA with ATF1 and the agent to be tested. The gels are run electrophoretically to determine which compounds result in disruption of a shift or supershift of the DNA. Larger molecules shift to a higher position on the gel and each complex produces a different and unique pattern. The use of the TFDA to identify an inhibitory agent of this invention, as exemplified by MAb4, is described in Example 3.

### Example 3

### Binding of ATF1 Mab4 Inhibits DNA Binding

Double-stranded oligonucleotides used in the electrophoretic mobility shift assays, obtained from Promega were as follows: CRE: 5′-AGAGATTGCC TGACGTCA GAGAGCTAG-3′ (SEQ ID NO:4) (CRE Catalog #E3281), AP1: 5′-CGCTTGA TGAGTCA GCCGGAA-3′ (SEQ ID NO:5) (AP1 Catalog #E3201). DNA binding mixtures (20 μl containing 10-20 ng recombinant ATF1 and/or CREB, 1 μg poly [dI-dC], and 2.5 μg bovine serum albumin in 10 mM Tris, pH 7.5, 50 mM NaCl, 0.5 mM DTT, 0.5 mM EDTA, 1 mM MgCl₂, 4% (by volume) glycerol, and 0.035 picomoles ³²P-labeled probe) were incubated for 20 min at room temperature, then run on native 4% polyacrylamide gels in high ionic strength buffer (25 mM Tris, 190 mM glycine, 1 mM EDTA) at 4° C.

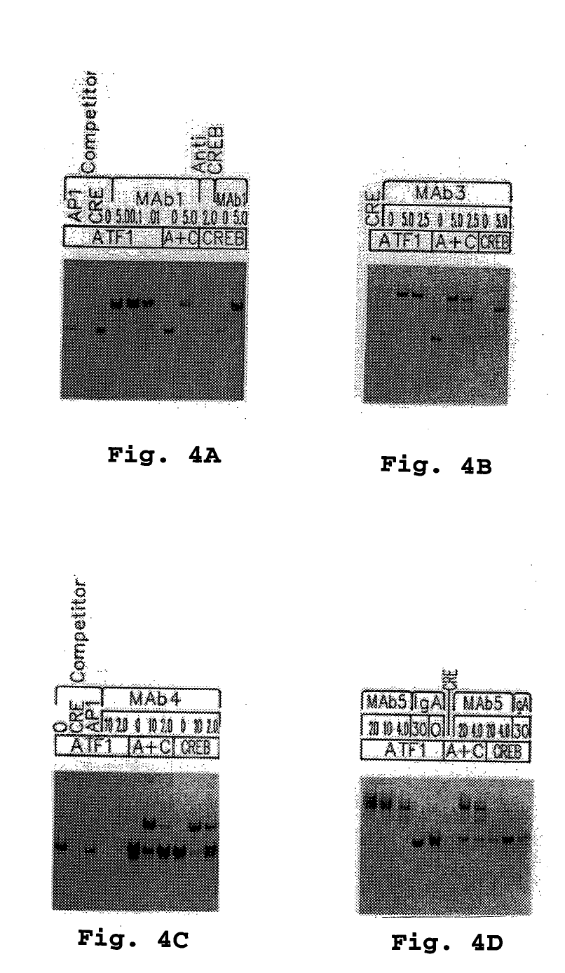

DNA binding assays with recombinant ATF1 and CREB (FIG. 4) demonstrated that MAb1 supershifts both ATF1 and CREB complexes to the same extent, and MAb3 shifts CREB a lesser distance than ATF1. MAb4 prevented ATF1-DNA binding, even if it was added after the DNA probe, but supershifted CREB. MAb5 supershifted ATF1 and did not react with recombinant CREB.

Decreasing amounts of each MAb were used in the DNA binding assay to determine ATF1 affinity. MAb1 has the highest affinity in this assay, with 0.020 μg of MAb (0.5:1 molar ratio of divalent antibody molecule to ATF1 monomer) completely supershifting 0.010 μg of ATF1. Two μg of MAb3 (50:1 molar ratio) or 5 μg of MAb5 (100:1 molar ratio) supershifted ATF1 to the slower migrating band (Supershift II) and 0.5 μg of MAb4 (12:1 molar ratio) completely prevented 0.010 μg of ATF1 from binding to the probe. Limiting amounts of MAb3 or 5 with ATF1 produced a faster migrating shifted band (Supershift I) at the same mobility as the MAb1 ATF1/CREB supershift or the MAb3 or 4 CREB supershift. Shifting all of the ATF1 to at least this level required 0.05 μg of MAb3 (1:1 molar ratio) or 0.20 μg of MAb5 (4:1 molar ratio).

Although not wishing to be bound by theory, it is believed that Supershift I represents one antibody molecule bound to each transcription factor dimer and Supershift II represents two molecules bound to each transcription factor dimer. A tenfold higher concentration of MAb1 and fifty-fold higher concentrations of MAb3 and 4 were required for CREB supershifts as compared to ATF1 supershifts or ATF1-DNA complex blocking. Reaction of MAb3 and 4 with CREB in the DNA binding assay was surprising because these antibodies did not react with CREB on dot blots, even if CREB was pre-incubated with unlabeled CRE oligonucleotide.

Results of preliminary DNA binding experiments with HeLa cell extracts demonstrated that MAb1, 3 and 5 supershifted most of the CRE binding protein. MAb3 or 5 (5-10 μg) produced two shifted complexes and a small amount of unshifted complex remained in reaction mixtures containing MAb5. Because of the high level of ATF1 produced, most of the CREB in HeLa nuclear extracts exists as ATF1-CREB heterodimers (Hurst et al., 1991). Again not wishing to be bound by theory, it is believed the MAb5 supershifted complexes represent ATF1 homodimers and ATF1-CREB heterodimers, and the unshifted material represents CREB-CREB homodimers. MAb4 reduced the total amount of shifted complexes, indicating that it prevents cellular ATF1 binding and may shift or prevent heterodimer binding, depending on the relative amount of antibody and ATF1 and CREB homo- and heterodimers in the solution.

### Example 4

### PCNA In-Vitro Transcription

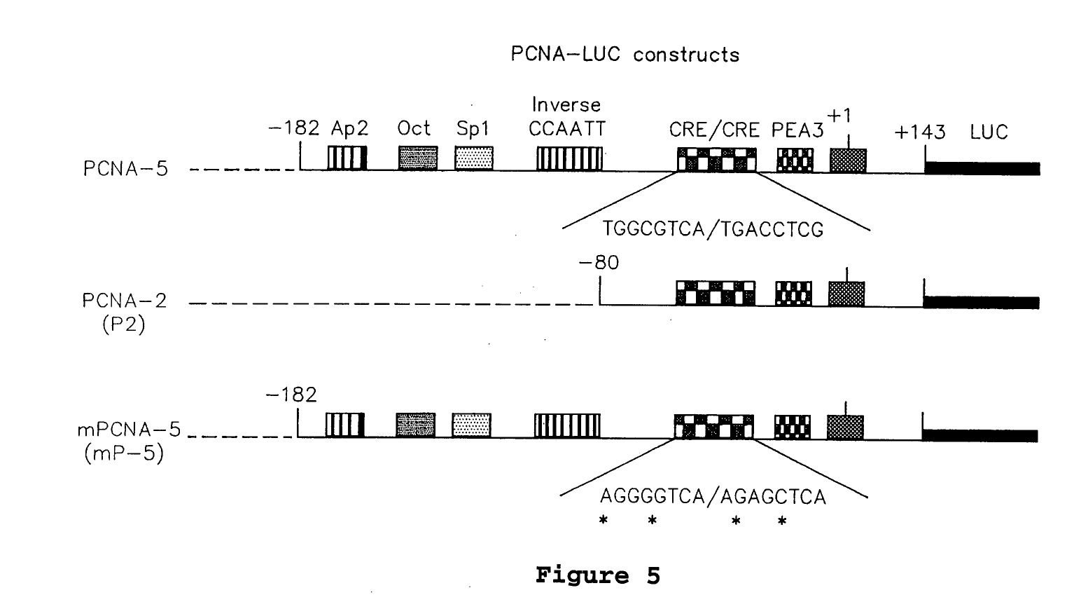

Effects of ATF1 MAb on transcription were evaluated using the HeLa nuclear extract in vitro transcription system from Promega according to the manufacturer's instructions (Promega, 1992) except that amounts of MgCl₂ (5 mM) and rATP (0.30 mM) were optimized as described by Farnham and Schimke (1986) and reactions were incubated at 26° C. for 1 hr. Antibody was incubated with nuclear extract and MgCl₂, for 30 min before adding rNTP's and template. Promoter templates (FIG. 5) were Proliferating Cell Nuclear Antigen (PCNA) luciferase expression vector constructs. PCNA 5 contains −182 to +143 of the PCNA promoter, PCNA 2 is a truncated construct containing only the CRE/CRE and PEA3 sites (−80 to +143) and PCNA-5 is a PCNA-5 construct with both CRE elements mutated

Specific PCNA RNA transcription was detected using the ³²P labeled primer

5′-GACTAGATGAGAGCTACTCTAAGAGGAACG-3′ (SEQ ID NO:6)

(EMBL Data Library, Accession=X53068) antisense to +97 to +127 of the PCNA gene (Shipman-Appasamy et al., 1991), prepared in accordance with the methodologies of Beaucage and Caruthers, 1981; and Sinha et al., 1983). RNA transcripts were annealed with the primer in 10 mM Tris-HCl, pH 8, 1 mM EDTA, at 70-75° C. for 10 min and cooled to room temperature for 10 min.

Reverse transcriptase buffer provided by the manufacturer was added and the solution was adjusted to 0.01 mM dithiothreitol, and 0.5 mM each of dATP, dTTP, dGTP, dCTP. Each 30 μl reaction was warmed to 42° C., 1 μl containing 200 units of SUPERSCRIPTO™ RNASE H⁻ reverse transcriptase (BRL) was added, and incubated for 30 min. Denaturing gel buffer, 20 μl, (98% formamide, 10 mM EDTA, 0.1% each xylene cyanol and bromophenyl blue) was added, samples were heated to 90° C. for 10 min and analyzed by electrophoresis on 6% acrylamide gels containing 7 M urea in 90 mM Tris-borate, 1 mM EDTA.

The labeled 127 bp product was sized by comparison with [symbol] X174 HinfI molecular weight markers from Promega (Catalog #E3511) and quantitated on dried gels with a Betascope 603 Blot Analyzer (Betagen Corp., Walthan, Mass., 1989) according to the manufacturer's instructions.

The effects of the panel of MAb on in vitro transcription using the murine proliferating cell nuclear antigen (PCNA) gene promoter as template were evaluated. The PCNA protein is expressed at much higher levels in proliferating cells than in quiescent cells, and is a co-factor for DNA polymerase delta, functioning in DNA replication during S phase. PCNA RNA transcription increases in interleukin-2 (IL-2) stimulated T cells during G1 phase progression, but PCNA mRNA levels are regulated by changes in mRNA stability in serum stimulated murine 3T3 fibroblasts (Shipman-Appasamy et al., 1991).

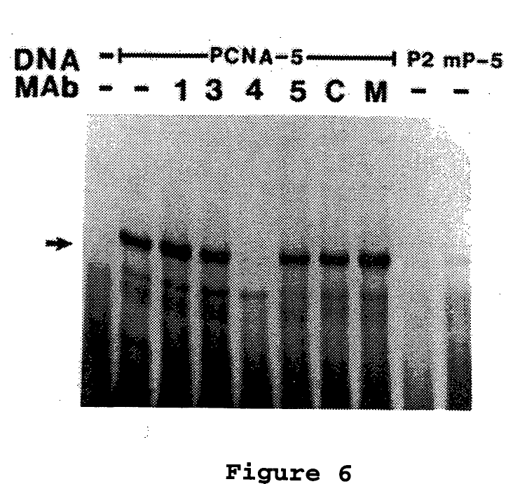

When added to HeLa cell nuclear extracts in the PCNA in vitro transcription system, MAb4 reduced transcription to 5% of reactions with no added antibody, MAb1 increased transcription 1.5-fold and MAb3, 5 or control antibodies did not significantly affect transcription (FIG. 6). In preliminary experiments with murine cell nuclear extracts, MAb4 also inhibited transcription. Transcription was reduced to 6% with a template containing mutated CRE elements, and was not detectable with a truncated template containing only CRE and PEA3 elements. Addition of MAb4 at approximately the same molar ratio as that required to prevent ATF1-DNA binding (12:1 molar ratio of divalent MAb to monomeric ATF1) reduced specific in vitro transcription to the same extent as mutating the CRE elements.

### Example 5

### Epitope Mapping

Because each MAb produces a different pattern in the DNA binding assay and two MAbs (#1 and #4) have opposite effects on in vitro transcription, the location of the MAb epitopes within the ATF1 molecule was determined. The first step in determining the fine specificity of the MAb was to cleave recombinant ATF1 into large fragments.

Testing several enzymatic and chemical cleavage methods determined that the best results were obtained with thrombin digestion. Two major cleavage products, with apparent molecular weights on SDS-PAGE of 22 kD and 14 kD, were produced.

For MAb epitope mapping, >95% pure (by SDS-PAGE) recombinant ATF1, purified on a DNA-cellulose column, was digested for 40 or 80 hours with human thrombin (3806 NIH units/mg, Calbiochem catalog #60S195) in 50 mM Tris pH 8.0, 5 mM EDTA, 1 mM dithiothreitol at 37° C., adding 0.4-1 unit of thrombin at 8-24 hour intervals. Digests were analyzed by SDS-PAGE and western immunoblotting and major proteolytic fragments were identified by protein sequencing from electroblots as described by Matsudaira (1987). The 8 amino terminal amino acids of each fragment were determined and compared with the known ATF1 sequence. The 22 kD fragment contained the amino terminus of ATF1 described by Yoshimura et al. (1990) and Rehfuss et al. (1991). The amino terminal sequence of the 14 kD fragment indicated that it was the carboxy terminal portion of ATF1 and that the major thrombin digestion site is after arginine 144 in the partial sequence described by Hai et al. (1989).

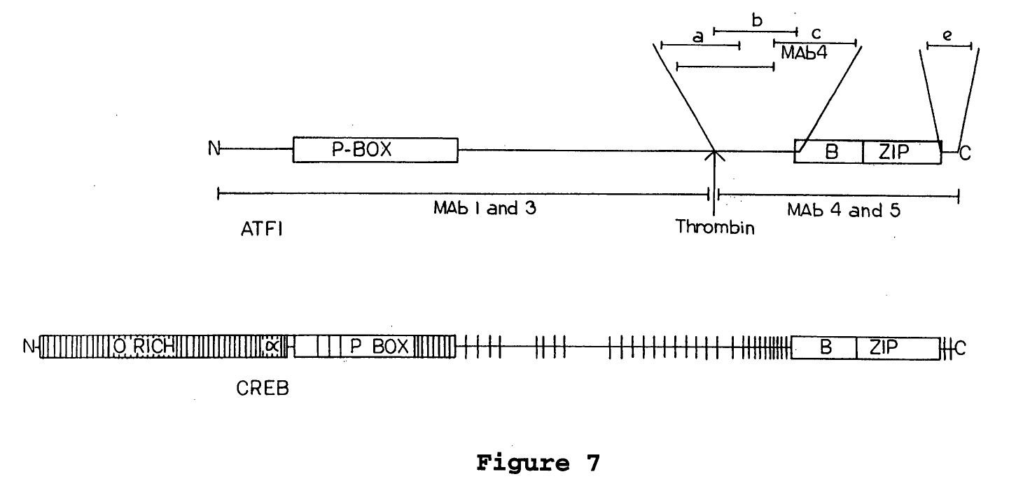

Immunoblotting and DNA binding analysis of thrombin digested ATF1 indicated that MAb1 and MAb3 react with the amino-terminal half of the molecule which contains domains involved in transcriptional activation (FIG. 7) and MAb4 and MAb5 reacted with the carboxy-terminal half which includes the leucine zipper and DNA binding region.

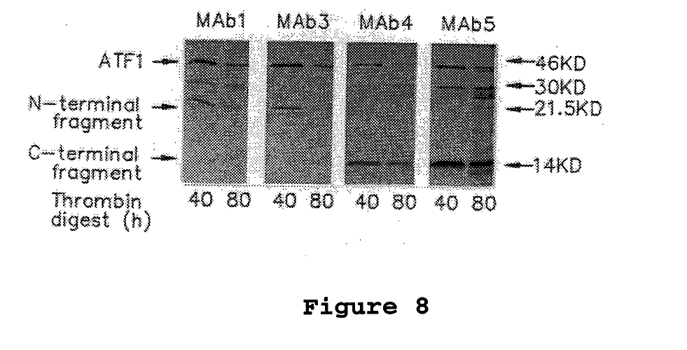

MAb1, MAb4 and MAb5 also react with a less abundant 29 kD fragment which does not react with MAb3 (FIG. 8). This 29 kD fragment may be produced when ATF1 is digested at a consensus thrombin site within the P-box, removing 78 amino terminal amino acids. Reaction of this fragment with MAb1 but not MAb3 indicates that MAb3 reacts with the amino terminal region, and MAb1 reacts with a centrally localized epitope on ATF1.

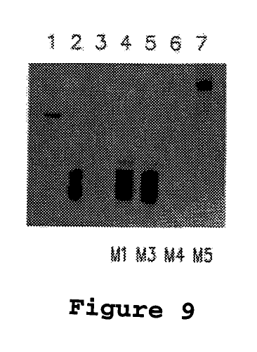

Identity of the major fragments was confirmed by DNA binding analysis (FIG. 9). MAb1 and MAb3 did not affect fragment-DNA binding, MAb4 prevented binding, and MAb5 supershifted bound fragments. Concentrating on the shorter 14 kD DNA binding fragment, overlapping synthetic peptides were produced, representing the areas within this fragment that diverge between ATF1 and CREB.

### Example 6

### ATF1 MAb Reactivity with Peptide c

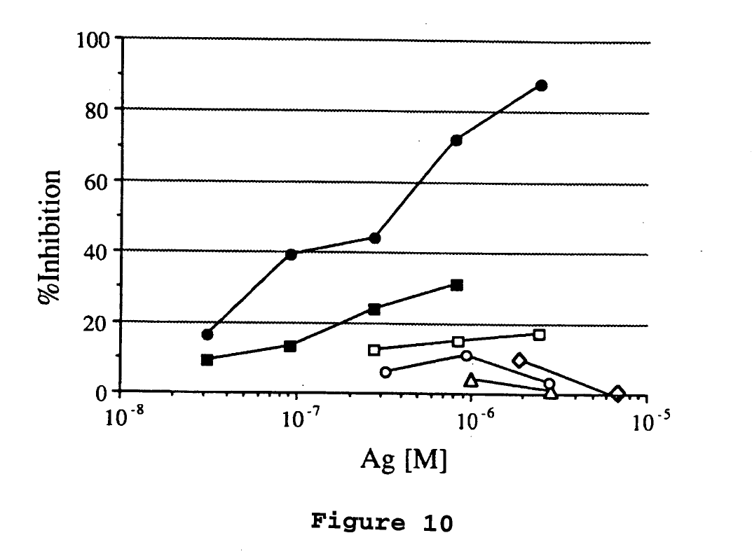

MAb4 and 5 reactivity was analyzed by dot immunoblotting and competitive ELISA. Focusing on the shorter 14 kD DNA binding fragment which reacts with MAb4 and MAb5, overlapping synthetic peptides representing the areas within this fragment that diverge between ATF1 and CREB were produced (FIG. 7). Peptides were synthesized via Fmoc procedures on a p-hydroxymethylphenoxymethyl polystyrene (HMP) resin support. After synthesis and oxidation the peptides were deprotected and cleaved from the resin by standard acidolysis in trifluoracetic acid and purified by reverse-phase HPLC methods. In FIG. 10, peptides represent the following ATF1 amino acids: a(▴): TTPSATSLPQTVVMT (residues 183-197 of SEQ ID NO:1); b(∘): VVMTSPVTLTSQTTK (residues 194-208 of SEQ ID NO:1); c(●): QTTKTDDPQLKREIR (residues 205-219 of SEQ ID NO:1); d(♦): PSATSLPQTVVMTSPVTLTS (residues 185-204 of SEQ ID NO:1); and e(□): EELKTLKDLYSNKSV (residues 257-271 of SEQ ID NO:1). MAb4 and MAb5 reactivity was analyzed by dot immunoblotting and competitive ELISA. On the dot blots, MAb4 reacted strongly with peptide c and MAb5 reacted weakly with peptide d.

In the competitive ELISA, peptide c inhibited MAb4 binding to ATF1 even more efficiently than the intact ATF1 protein (▪ FIG. 10). The other peptides did not affect MAb4 binding. None of the peptides inhibited MAb5 binding to ATF1 in ELISA. These assays identified the MAb4 epitope within the 10 amino acids amino-proximal to the DNA binding region (amino acids 205-219, peptide c). However, although the MAb5 epitope may be within peptide d, it is not accurately represented by the synthetic peptide and may be similar to a discontinuous epitope described by Szilvay et al. (1993).

### Example 7

### Cloning and Screening of sFv

The single chain Fv of mAb4 was cloned utilizing the procedures as originally described by Winter and Milstein (1991), with modifications as described below. Total RNA was isolated from the mAb41.4 hybridoma and reverse-primed with random hexamers. The use of random hexamers eliminated the need for Ig specific or oligo(dT) primers that require synthesis of long cDNAs. The resulting cDNAs were of sufficient length to clone the V regions. The heavy and light V regions were amplified in two separate reactions, using degenerate primers to the framework regions bracketing the CDRs of the V_{H} and V_{L} domains. The two PCR products were linked together with a DNA linker. The linker DNA was designed such that it overlapped the 5′ end of the V_{L} PCR product, and the 3′ end of the V_{H} PCR product, to result in sFv cDNA encoding V_{H}-link-V_{L} that was subsequently cloned into the NotI and SfiI sites of the pCANTAB phagemid vector (provided by Dr. S. Paul, UNMC). This vector places the sFv upstream of a His-6 tail and c-myc antigen tag as well as the M13-g3 protein providing for purification and detection. Expression of the vector in E. coli TG-1 plus the presence of the M13-K07 helper phage results in the production of sFv-g3 fusion protein to give a phage surface displayed sFv. Phage capable of binding ATF-1 or CREB were screened by ELISAs using recombinant ATF-1 bound to the microtiter wells. Positive wells were detected with a conjugated anti-M13 antibody. Those phage found to bind to transcription factors were used to infect E. coli HB2151 to generate periplasmic soluble sFv. This method is suitable for the screening of any antibody capable of binding to the claimed region in any b-ZIP transcription factor.

### Example 8

### Production, Purification and Sequencing Results of the sFv

Soluble Fv was produced and quantitated as described by Gao and Paul (1995). E. coli HB2151 that had reached an A₆₀₀ of 0.6 were induced with 0.4 mM IPTG and grown at 25° C. for 4 hours. Periplasm was extracted in a high salt lysate buffer, clarified and dialyzed. Typical yields were 0.5 to 2.5 mg/L of culture. Quantitation of sFv was done by performing slot blotting and staining with an anti c-myc-tag antibody (murine 9E10 hybridoma, ATCC) and an AP-conjugated anti-mouse antibody (IgG H&L chain; Jackson Immuno-Research Laboratories, West Grove, Pa.). Densiometric analysis was performed using c-myc-peptide-1 (Oncogene Research Products) a standard curve was generated and the signal of sFv wells was read off of the curve. The crude periplasmic extract was used in protein binding studies, as well as purified through isoelectric focusing for more refined studies.

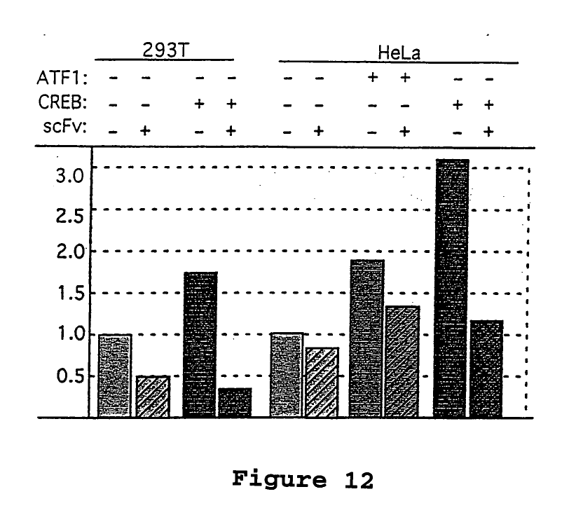

sFv clones were sequenced by the automated sequencing core facility at the Eppley Institute. Using the MacVector software package, the sequence data of three different clones were aligned to produce a consensus sequence and translated. The sequences are listed in SEQ ID NO:7 for the V_{H} region and SEQ ID NO:8 for the V_{L} region. Therefore, the composition of an example of a compound capable of the key feature of the invention is available. The protein sequences of the heavy and light chain variable domain are capable of binding to ATF1 and CREB. In the single gene described here as sFv4, these sequences are joined by a linker peptide (SEQ ID NO:9) to form a compound capable of inhibiting ATF1 and CREB activity. The DNA and translated protein sequences of the V_{H} and V_{L} regions were compared to genebank entries and the Kabat Antibody data base via internet provider. Results showed that the V_{L} was unique and shared homology with mouse Ig Kappa chain regions. The V_{H} sequence was also unique and shared homology with mouse heavy chain framework and variable regions. Comparison to the Kabat data base identified unique and unusual features of each V region, as well as identified the antibody family. The V_{L} region belongs to the Kappa-III family, and the V_{H} region belongs to a miscellaneous group but was most similar to the Ig III subfamily with CDR3 deleted. The Kabat database also identified the framework regions and CDRs of the V_{L} and V_{H} sequences which are listed as SEQ ID NO:8 and SEQ ID NO:7, respectively. The amino acid sequence of the sFv Fv regions have been compared to the available mAb sequence obtained using an automated protein sequencer. The first 55 amino acids for the V_{L} extending from FR1 through CDR2 are identical to that obtained for the corresponding region in the mAb4 IgG. To better understand which CDRs of the sFv were contacting the epitope on ATF-1, molecular modeling of the sFv was performed and is shown in FIG. 12. Amino acid sequences of the V_{H} and V_{L} were analyzed by the Glaxo Swiss-Protein database for best fit alignment to known crystallized Fv structures.

### Example 9

### sFv Inhibition of ATF-1 and CREB Binding to DNA

Electrophoretic mobility shift assays (EMSA) were performed using 20 femtoM ³²P-labeled oligonucleotide containing a consensus CRE:5′-AGA GAT TGC CTC ACG TCA GAG AGC TAG-3′ (SEQ ID NO:4), and 50 ng recombinant ATF-1, or CREB in the presence of Mab4, Fab or sFv periplasm or a mock periplasmic extract prepared identically to sFv periplasm except lacking sFv, in 20 uL reactions containing 1 ug poly(dI-dC), 50 mM NaCl, 0.5 mM DTT, 0.5 mM EDTA, 1 mM MgCl₂ and 4% glycerol. Reactions were incubated for one hour at 37° C. and electrophoresed at 25 miliamperes for 1.5 hr at 4° C. on native polyacrylamide, 1.5 cm gels in high ionic strength buffer (25 mM Tris, 190 mM glycine, and 1 mM EDTA). Bound and unbound oligo were detected by autoradiography for 6-12 hours on a phosphorscreen.

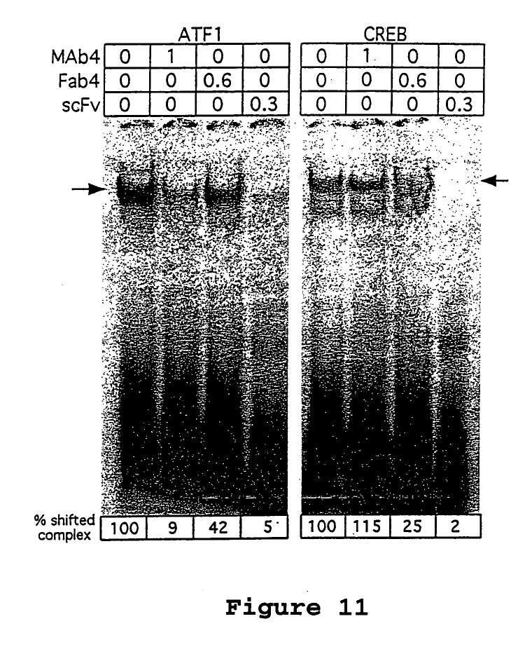

A representative experiment demonstrating discovery of the inhibitory nature of the sFv4 protein for either ATF1 or CREB is shown in FIG. 11. Procedures were conducted using EMSA in which the proteins binding to DNA are visualized by using radioactive DNA sequences containing the site to which CREB and ATF1 adhere. If CREB or ATF1 are bound to the DNA, its migration through a gel is retarded, resulting in a band which is detected on X-ray film or an imaging machine, whereas the remaining non-bound DNA migrates to the bottom of the gel. Inhibition of the complex formation between DNA and ATF1 or CREB is noted by the reduction in band intensity. The reduction is measured by densitometry. An experiment with ATF1 and CRE-DNA and ATF1CREB is shown in the left panel, with the effect of sFv compared to that occurring with Mab4. The arrow indicates the location of the ATF1 or CREB complex. The panel on the right shows CREB and CRE-DNA and either sFv, Fab or Mab4. Boxes at the bottom of panels indicate the amount of complex remaining after addition of either sFv, Fab or Mab4. Note the near complete elimination of complex at the arrow, resulting from the addition of sFv. This result demonstrates the essential aspect of the invention whereby an inhibitory protein is able to eliminate the DNA binding activity of ATF1 and or CREB.

### Example 10

### Intracellular Expression of sFv Interferes with CRE-Driven Gene Expression

Transient cotransfection assays in cells were performed to determine if expression of the sFv could interfere with expression of a CMV-IE luciferase reporter. The measurement of inhibition is accomplished by co-transfection of a reporter capable of expressing the luciferase (luc) protein and a construct expressing the sFv. In the absence of sFv, the luciferase gene can be expressed and detected by the activity of the luciferase protein. The goal of this study was to demonstrate that the inhibitory mechanism was not only effective in vitro but would occur in living cells derived from cancerous tissues. Two sFv expressing constructs were utilized, pCMV-sFv and pEF-sFv. These two expression vectors were obtained by placing the sFv cDNA into the poly-cloning sites of pCMV4 and pEF-1 (provided by Dr. R. Lewis, Epply Inst.). pCMV4 is a powerful expression vector that incorporates the SV40 ori, and the translational enhancer from Alfalfa Mosaic Virus 4, in addition to the CMV-IE promoter. pEF-1 is a derivative of this vector that has the CMV-IE promoter replaced with the EF-1 alpha promoter. The transient cotransfection experiments were performed in the presence and absence of ATF and CREB, also supplied via transfection. ATF-1 and CREB cDNAs were inserted into the pCMV4 vector for these experiments. Transient co-transfections were performed according to established protocols (Example 1), using either 293T cells or HeLa cells with the calcium phosphate precipitation or DEAE dextran technique, respectively. The transfections were performed in duplicate with 2 ug of reporter construct (CMV-Luc), 4 ug of CMV-ATF1 or CMV-CREB, and 4 ug of sFv vector (either CMV-sFv or EF-sFv) for 2, 3.5 mm wells. In the control assays without sFv, a molar equivalent of parent vector was used (without sFv insert) to maintain an equal number of promoter units in each transfection. 292T cells were harvested at 48 hours post-transfection and HeLa cells were harvested 72 hours post transfection.

The reporter system utilized the measurement of firefly luciferase according to established protocol (Ausubel, F. M., et al., 1992). Following transfection, cells were harvested in the presence of Triton X-100, and ATP and luciferin were added and light output was measured with a luminometer (Analytical Luminescence Laboratories, Ann Arbor, Mich.). Results of three experiments were normalized with the reporter construct expression result set to 1.

Results show that the sFv is capable of reducing reporter gene expression (FIG. 12). The height of the bar indicates the relative activity of the luciferase construct in paired experiments with or without sFv4. The presence of sFv reduced overall luciferase activity by 50% in 292T cells and inhibited the CREB activity by 300% both in 293T and HeLa cells. When cotransfected with pCMV-ATF or pCMV-CREB, the observed amplification of luciferase expression, that was due to either the ATF-1 or CREB, was returned to levels similar to or lower than reporter alone. Thus, not only ATF1 induced expression was reduced, but CREB induced expression as well. It is possible and likely that other b-ZIP transcription factors, discovered and undiscovered, also contributed to expression as measured in this system. This demonstrates that the subject of this invention has in vivo activity sufficient to cause reduction in transcription through factors in the b-ZIP transcription factor family. This also demonstrates that the inhibitory effect occurs in living cells derived from cancerous tissues.

### Example 11

### Interference With Viral Activity by a Compound With Structure Present in mAb4

Several models have been described in the literature for the interaction of Tax and transcription factors. One of the most plausible explanations of Tax activity is that Tax dimers stabilize the binding of CREB to TRE and CRE sequences (Tie et al., 1996; Baranger et al., 1995). The current model suggests two molecules of Tax contact the two α helices of CREB and ATF1 as they emerge from the major groove on opposite sides of the helix; a major site of contact being the 282-284 AAR residues of Tax (Tie et al., 1996). Tax is a 40 kD protein, and in the absence of structural information it is not possible to predict how this occurs; however, the distance between the a helices of a b-ZIP protein at the point they emerge from the major grooves is approximately 30A, and they diverge at an angle of at least 30°. Therefore, at least a portion of Tax could contact ATF1 or CREB or other b-ZIP transcription factors near the site of interaction with the compounds described in this invention. If the dissociation constant of the sFv for CREB is less than or equal to that of Tax for CREB, then the sFv could displace Tax in the site of interaction with CREB, eliminating the ability of the virus to induce disease. The alternative mechanism is that, since b-ZIP transcription factors are continually cycling on and off DNA, the inhibitory molecule could bind to the transcription factor and prevent rebinding of the factor to DNA.

Electrophoretic shift assays were performed as described in Example 9, with the following modification. The radio labeled DNA used was a portion of the HTLV-I regulatory element that contains the Tax responsive element (TRE). The TRE is similar in sequence to the classical CRE sequence. Each lane contained equal amounts of radio labeled TRE DNA (20 femtoM) and 50 ng of recombinant CREB protein and approximately 400 nM of recombinant Tax protein, an amount previously determined to enhance the CREB-TRE DNA complex formation.

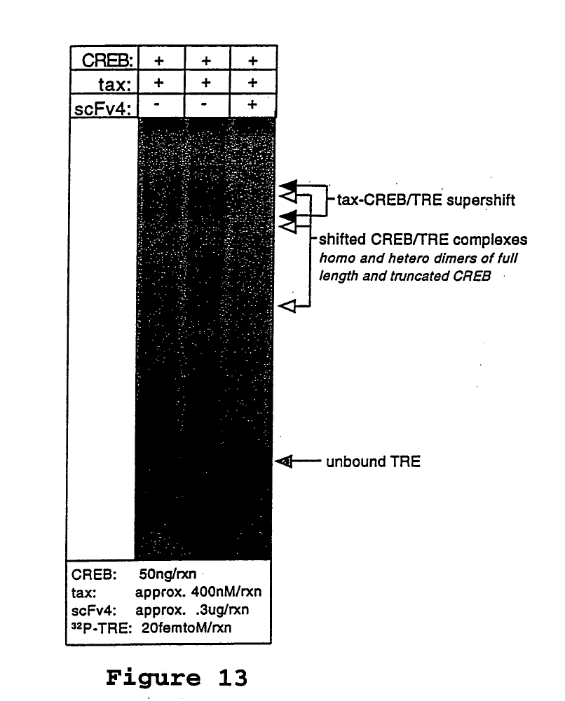

Demonstration of the ability of the present invention to inhibit the activity of the viral HTLV-I Tax protein was measured by electromobility shift assay as shown in FIG. 13. Lane 3 contained 0.3 ug of sFv, whereas the first two lanes contained periplasmic extract to control for potential non-specific activity. The natural Tax effect is recognized by the enhancement of band intensity (presence of dark bands) in the first two lanes. The effect of the invention is demonstrated by the loss of band intensity in lane three which results from the addition of sFv. This result demonstrates that the invention's activities dominate the activity of the virus in that the sFv was able to inhibit the Tax enhancement of the CREB protein binding to DNA.

### Example 12

### Dissociation and Rate Constants for Antibody Interactions with CREB and ATF1

Determination of CREB and ATF1 equilibrium constants: Native ATF1 and CREB lack tryptophane and therefore tyrosine fluorescence is excited with a N2 laser and the changes of the fluorescence lifetime followed, yielding the equilibrium constant. Alternatively, HPLC and frontal zone analysis are employed with observation at 220 nM to determine the equilibrium constant, if this equilibrium; on the basis of gel electrophoresis, appears rapid. Once the equilibrium constant is determined, dilution jump experiments (adaption of Metallo and Schepartz, 1997) are carried out to yield the rate constants, or establish a lower bound for the equilibrations. If the reactions are in the sub-msec time frame, then the process is treated as an equilibrium process in all subsequent fittings.

Determination of binding constants and rate constants: The consensus CRE element (“DNA”) with flanking sequences from the somatostatin promoter was synthesized with fluorescein at the 3′ terminus bases (5′-GCCTGACGTCACCG-3′ fluorescein) (SEQ ID NO:13). Binding constants are obtained by measuring fluorescence polarization as a function of both transcription factor and DNA, since the two equilibria are coupled. From known values for the two equilibrium constants, it is straightforward to obtain the rate constant for association of transcription factor binding to DNA. If studies are conducted at high concentrations of ATF1 or CREB, monomeric forms are negligible. After determining the first constant, ATF1 and CREB is then reduced to the concentration range where monomers are abundant and the association rate constant for transcription factor dimerization from the coupled kinetics is obtained using the anisotropy change for the second step as the marker event.

sFv contains Trp, and thus fluorescence intensity, lifetimes, and polarization (anisotropy) are measured as a function of sFv concentration, and HPLC frontal zone analysis is used either as an alternative to fluorescence or to confirm the fluorescence data, or to discern whether further aggregation is possible. sFv does not bind directly to DNA at either the CRE or TRE element. Strategies are available to determine the rate constants involved in this process. If the reaction half time is on the time scale of 1 sec, a simple dilution jump stopped-flow experiment is used. Peak-shape analysis of the HPLC eluant is used if the rates are even faster. Knowing the binding constant, the assumption of a diffusion limited association process yields an upper limit for the dissociation rate constant. For processes on the scale of 10 usec to a few msec, temperature-jump measurements together with fluorescence detection is used. In an alternative approach, this process is coupled to the rate of transcription factor (or peptide) binding.

The ATF1 binding region for sFv was modeled by peptide c, which was prepared by standard procedures with fluorescein (F*) distant from the epitope: F*-SQTTKTDDPQLKREIR (residues 204-219 of SEQ ID NO:1). This labeled peptide is titrated with sFv as a function of (sFv), and the binding constants for sFv binding to the equivalent of monomeric ATF1 or CREB are determined. Flow of F*-peptide vs. sFv is compared to obtain the rate constants for the above reaction step. Binding constants for the above equilibria are extracted purely from fluorescence. Once the prior equilibria are determined, there are only two other thermodynamically independent equilibria required to establish the energetics of the interactions of sFv with ATF1:
ATF1 dimer+sFv 2ATF1.sFv;
ATF1 dimer.sFv+sFv 2ATF1.(sFv)2.

As discussed above, concentrations of sFv (nM) where multimers do not form are used, however the binding of two separate sFv's is possible to each ATF1/DNA complex. Knowing the previous equilibria, high concentrations of transcription factor where dimer is predominant are used to obtain these two equilibria by following Trp fluorescence intensities, polarizations, or lifetimes. After confirming that ATF1 is dimeric at the experimental conditions, the two required association constants are obtained in a stopped-flow rapid mixing experiment and the thermodynamics for the interactions of sFv (one or two molecules) with ATF1 (monomeric and dimeric) are established.

The first association constant is determined by poising the system (knowing all of the equilibrium constants as outlined above) so ATF1.sFv is the dominant species, and flowing fluorescently labeled sFv against the former solution. All other rate constants for all other paths are known, except for the process where ATF1 existed in either dimeric or monomeric form, which is not relevant under the experimental conditions. Then, moving to much higher concentrations of sFv it is determined whether the dimeric or monomeric form predominates. ATF1 is labeled at lysines with NHS-fluorescein, which should not interfere with the binding to the ATF1 by sFv.

Investigation of sFv multimerization in binding specificity. A determination of whether multimers of sFv are a factor involved in the activity of the sFv with CREB is made using fluorescent techniques as outlined above. One explanation for the reactivity of sFv4 for CREB is that a reduction in size of the antibody allows contact of the binding domain to the epitope that was not previously available. A second consideration is that formation of multimers by the sFv results in apparent affinity due to increase in avidity (Whitlow et al., 1994). Concentrations of sFv as low as 10 nM were capable of showing inhibition of CREB complexes on gel shift which is significantly less that the concentration at which aggregates dissolve into solution (5 mg/ml). Therefore, affinity of sFv4 for CREB is not believed to be due to formation of multimers. This is confirmed by determining the relevant equilibrium constant and hence the percentage of sFv that exists in aggregated form at the concentrations which were employed in gel shift experiments.

Thermodynamics and kinetics for the complete reaction scheme. In order to elucidate the thermodynamics and kinetics for species involving 2 ATF1 bound to CRE-DNA the system is poised toward ATF1dimer bound to DNA (where the DNA is fluorescently labeled) and changes in fluorescence anisotropy as sFv is added are followed. The equilibrium scheme and determination of the two kinetic steps is represented by the reaction,
ATF1 dimer/DNA⇄ATF1dimer/sFV+DNA.
Since the changes in molecular weights of the complexes are large, the anisotropies are known for all species, and all equilibrium constants are known for fitting fluorescein lifetime or anisotropy data for any combination of the three reagents. The reactions are isolated by using energy transfer because energy transfer from Trp of sFv to a pyrene attached to the 3′ or 5′ of DNA will only occur in the species ATF1 DNA. These results determine the energetics of how sFv binding to ATF1 alters the affinity for DNA. The rate constants in the above scheme are then determined for a detailed understanding of the mechanism of importance in determining the mechanism of sFv inhibition. The binding constants of sFv to both ATF1 and to ATF1/DNA are completely determined from thermodynamic dependence. The remaining rate constant for separation of DNA from ATF1 is determined by poising the system so the initial concentrations of all species are known, and in particular, the species ATF1/sFv can be made dominant as sFv is varied. Various amounts of those solutions are then flowed against labeled DNA and the kinetics followed both by anisotropy of DNA as well as by energy transfer from Trp of sFv to pyrene attached to DNA. The same strategies are used for binding of sFv or other derivatives to CREB since the relevant equilibrium and rate constants for dimerization of CREB are established.

If difficulties arise in obtaining equilibria at even high concentrations of ATF1 or sFv in the stop flow reactions, chemical modifications can be used to assist in dissecting the above mechanism by cross-linking 2 ATF1 (or CREB) at the distal end of the b-ZIP domain to assure that only dimers of ATF1 or CREB are present in solutions. Flowing DNA against this solution assures the measurement of the association constant for formation of ATF1 or CREB and DNA. Studies will be carried out with non-phosphorylated CREB and ATF1. Having established the rate constants for the non-phosphorylated forms, it is possible to explore in detail how transcription might be regulated by phosphorylation in this system, since it is known that phosphorylation differentially affects transcriptional activation of ATF1 and CREB (Gonzalez and Montminy, 1989). Such studies provide the basis for evaluating improvements in newly derived sFv's. Additionally, these data provide biophysical evidence for the mechanism of action of the sFv and provide support for the rational design of sFv's which selectively bind to b-ZIP transcription factors.

### Example 13

### Determination of contact residues between sFv4 and ATF1 and CRER

Modeling according to Antibody Modeling (AbM) Protocol. The primary sequence of the variable heavy and light chains of mAb4 was determined. From the sequence information, modeling of the antibody CDR's can be performed with the commercial version of AbM v2.0 (Oxford Molecular Ltd). The modeling program can be utilized prior to substitution studies (described below) to investigate the effect of replacing or deleting antibody residues predicted to play an important role in binding to antigen. The structural effect of replacing residues with alanines is investigated by examining gross alterations in CDR structure as determined by the program. The AbM protocol takes a holistic view of available antibody construction methods and utilizes canonical structures, database and conformational searching, or a combination of the database approach with conformational searching where appropriate (Martin et al., 1989). This approach takes advantage of crystallographic information and maintains the ability to saturate space using ab initio methods. The binding site in Fv is a β-barrel formed from VH and VL anti-parallel β-sheets. Five of the six CDR loops adopt canonical conformations determined by H-bonding, packing arrangement or backbone torsional angles in a few residues in a loop of a defined length (occasionally including FR residues). On average, the surface area of the antigen-binding site and the epitope contact surface occupies a surface area ranging from 400 to 600 Å² squared. Of particular relevance for this study is the three dimensional structure of an mAb and synthetic peptide antigen of myohemerythrin (Mhr)(Stanfield et al., 1990). Since the specific epitope has been discovered, rapid recognition was possible from the electron density maps of contacts between the CDR and peptide epitope. Seven peptide residues were identified from four CDRs as composing the contact surfaces. The residues of the bound peptide were those as expected based on previous immunological assays and replacement studies showed that three residues were essential for binding. The most significant finding was the identification of a conformational change occurring in the peptide upon binding to the Fab. The AbM program builds the most conserved regions of the V-domain (FRs) by comparison with the most homologous antibody structure in the Brookhaven databank, PDB (Martin et al., 1989; and Chothia and Lesk. 1987). Next, canonical CDR loops (CDRL1-L3, CDRH1, CDRH2) are placed onto the framework. Although CDRH3 is typically constructed by searching for all entries in the databank for loops of the same length which satisfy the C-alpha distance constraints within 3.5 s.d, in this situation, the CDRH3 has been deleted. Initial reconstruction and side-chain addition is done by searching conformational space via rotation on a torsional grid about the f, y, and c torsional angles. Monte-Carlo simulated annealing is done where necessary. Modeled loops are ranked by an energy screening procedure using a solvent-modified Eureka force field. A conformation most similar to database conformations of “structure determining regions”, or, if such a conformation is not found, the lowest energy conformation is chosen.

This program can build antibody models within 3A RMS. The caveats are that data based on crystals of antibodies may not accurately reflect solution structures. Antibodies could exist in alternate conformational states (Schiffer et al., 1989; and Buchner et al., 1989) and antigen binding may induce conformational changes in the antibody, such as domain movements due to an induced-fit mechanism (Rihs et al., 1991; and Arevalo et al., 1993). Modeling studies identify mAb 4 solvent-accessible residues, the importance of which is then tested by comparison with structural motifs identified by x-ray diffraction. Following solution of the crystal structure, key residues involved in direct contacts with epitope are identified and alanine substitutions are performed to identify those residues predicted to have the greatest impact on binding. Up to five residues are selected to individually mutagenize through cloning methods described below.

Analysis of the CDR and epitope structure of the mAb 4 Fab or sFv and the ATF-1 peptide antigen complex by X-ray crystallography. Analysis of the CREB peptide sequence revealed that the transition to turn-like motifs is predicted to occur after 5 additional residues further NH₂-terminal than in ATF-1, which would result in a longer a-helical domain. It is of interest to determine if this extension of an alpha helical domain in CREB accounts for the decrease in affinity of mAb 4 for CREB. Structural studies of the Fab fragments of mAb 4 are performed in the presence of ATF-1 contact region peptide c. Analysis of the antibody-antigen complex provides a means to determine mAb 4 CDR residues important for antigen binding, evaluate if mAb 4 elicits a conformational change in ATF-1 upon binding, and determine structural differences between ATF-1 and CREB and other b-ZIP proteins.

Fab-peptide diffraction studies. X-ray crystallography is utilized to analyze the structure of mAb 4 Fab in the presence of the ATF-1 contact region of peptide c. Structural data is obtained for the interaction between the mAb4 antigen binding domains and the region of ATF1 comprising the relevant epitope. Overlapping peptides were generated and screened for ability to compete with full mAb4 as determined by competitive ELISA. One 15 residue peptide (peptide c) inhibited binding to recombinant ATF1 more efficiently than self-competition by the full length ATF1 protein. Adjacent peptides did not compete. These findings were supported by antigenic index analysis of this region and comparison with the CREB sequence that showed peptide c contained an antigenic region not present in CREB.

Fab production. Fab is used for generating crystals. Monoclonal antibody 4 is an IgG1 subclass and the classic method of preparation of subfragments utilizes papain. Several digestion protocols were evaluated for the generation of Fab from mAb4 including modifications of the commercially prepared immobilized ficin procedure (Pharmacia) (Mariani et al., 1991). We determined that papain (Sigma) 1 U/ml in 20 mM TBS, pH9.5 activated in 50 mM cysteine, 1.25 mM EDTA produced optimal digestion over 10 to 12 hr. Fab fragments are purified on protein A columns which remove Fc fragments. Confirmation of digestion is analyzed on SDS-PAGE gels and visualization with silver stain. Products are evaluated on reducing and non-reducing gels and blotted with light chain and Fc antibodies for confirmation of correct molecular size. Samples are concentrated, purified by size exclusion chromatography (30,000 M.W. cut-off), and cation exchange chromatography with a Mono-S column. Fractions collected of the appropriate size are dialyzed against 10 mM phosphate buffer, pH7.6. Sodium azide is added prior to storage.

Cystallization and data collection. X-ray diffraction studies and subsequent analysis are performed with the immediate goal of identifying contact residues between the Fab and peptide epitope. Conditions have been optimized that yielded crystals of the pentadecamer (peptide c) and an undecamer (peptide c3) using the hanging drop, microvapor diffusion method (McPherson, 1982). Crystallization is performed using multi-well plates (Stura and Wilson, 1994) in a constant temperature incubator at 22.5° C. The crystals of the Fab in the presence of the pentadecamer grow as needles. The preferred method for growing crystals is micro-seeding, although other methods known in the art can be used. Selected crystals have generated diffraction patterns consistent with antibodies with a resolution of approximately 2.0 Å. Crystal decay was a problem, however, requiring the merging of data from different crystals. A liquid nitrogen based low temperature device can be installed on the MARreserach detector to provide better quality data, or data can be collected using the Stanford synchrotron. X-ray diffraction data are collected using an 18 cm diameter MARresearch imaging plate area detector on a Siemens rotating anode X-ray generator. Data collection is controlled with the MARDC software provided by Area Detector Systems Corp. Data collection procedures are optimized by varying detector to crystal distance, scan range, and number of cycles per exposure. Data reduction is carried out with the MARXDS (X-ray research, Hamburg) software or with MOSFLM library using in-lab Silicon Graphics workstations. Software used for data analysis is known in the art and includes the CCP4 library (Daresbury), Xtal View (D. E. McRee), Merlot (P. M. D. Fitzgerald), X-PLOR (A. T. Bruger, Yale Univ) and DEMON (FMD Vellieux, IBS/ICCP, Grenoble). The initial phasing was accomplished by molecular replacement using the backbone and β-carbon atoms from known IgG-Fab crystal structures as starting models (Brookhaven Data Base). Electron density plots are displayed either with CHAIN or with INSIGHT II (Biosym Technologies, San Diego, Calif.). The high performance graphics workstations are used for stereoscopic display and fitting of electron density maps.

Molecular modeling and structure prediction is carried out using X-PLOR for crystal structure refinement. PROSA (Center of Applied Molecular Engineering, Universitat Salzburg) may be consulted to assess the quality of the model. DISCOVER may be used for protein modeling. After identification of the overall structure, electron density maps are displayed and visually inspected. Replacement and substitution experiments are performed to analyze the impact on peptide-Fab interactions. Contacts predicted to be non-essential may be studied further by simulated docking experiments. X-ray crystallography data can provide valuable information on the binding trajectory of the antigen: antibody interaction. The trajectory of the interaction takes into account not only secondary structure of the binding site but the “angle of approach” at which the interaction occurs. The results from these studies can be interpreted in light of our solution kinetics studies and suggest new rapid reaction experiments. By defining the topography of ATF-1 contact region c, this information will play a role in helping to determine the mAb 4 CDR residues important for ATF-1 binding.

Confirmation of key residues involved in binding of the sFv through mutagenesis and competitive ELISA. Following identification of key residues of the CDRs that have close interactions with peptide, confirmation of their importance is confirmed by site directed mutagenesis. Several approaches known in the art are available, however preferred is site directed mutagenesis and replacement of residues with alanine through inclusion of the mutated sequence in primers used in the PCR reaction. Mutagenized clones are sequenced for confirmation of correct replacement of the targeted residues. Confirmation of the importance of the mutagenized residues is determined by the demonstration of reduced affinity to ATF1. Effect upon affinity is evaluated using periplasmic extracts in our competitive ELISA procedure as previously described using recombinant ATF1 to coat microtitre plate wells (Orten et al., 1994). Competition is performed with peptide c which represents the mAb4 epitope of ATF1. Increasing concentrations of peptide are added to the solution containing sFv over a range from 0.01 μM to 1 μM and allowed to incubate. Detection of bound sFv is accomplished with the polyclonal goat anti-mouse Fab antibody and a peroxidase conjugated donkey anti-goat antibody. After addition of substrate the plate is read and results are plotted as percent inhibition of wells without competitor. Controls include periplasmic extract from a non-relevant sFv. Comparison of results are made with those obtained with the parental sFv4.

Amino acid residues within the CDR that contact the transcription factor epitope can be determined as described in Example 13. Furthermore, mutation studies can confirm which residues are essential for activity of the antibody and provide a basis for proposing substitutions for improving affinity and specificity. It is preferable to use the smallest possible sequence that is capable of being bound by mAb4. Churchill et al. have shown that a reduction in size from 30 to 6 residues significantly improved resolution, although only their 30 residue peptide was capable of initiating spontaneous nucleation and crystal growth (Churchill et al., 1994). Small co-crystals of 30 residue peptide and Fab were then used to seed solutions of smaller peptide epitope (Gao et al, 1995). A similar approach can be taken after obtaining crystals. Overlapping peptides of varied length can be generated and screened by competitive ELISA against recombinant transcription factor with mAb. Smaller peptides retaining 75% of the inhibitory activity can be selected for further analysis. Peptides are deprotected and cleaved from the resin by standard acidolysis in trifluoroacetic acid and purified by reverse-phase HPLC methods.

The example for determination of contact residues between sFv4 and ATF1 and CREB is offered by way of illustration and the same or similar procedures can be applied in the determination of contact residues of other sFvs.

### Example 14

### Generation of Improved Anti-Transcription Factor sFv Constructs

When the key residues of the CDRs that have close interactions with peptide and likely play a role in specificity of binding are known, a directed mutagenesis approach with oligonucleotides and PCR is preferred. Alternatively, random mutagenesis is utilized to generate derivatives of the sFv and screen them by competitive ELISA to identify mutants with the ability to bind CREB with greater specificity and higher affinity than sFv4. Following the procedure of Gao and Paul (1995) and Deng (1995), the first round of PCR utilizes forward primers that encode the amino acids to be substituted flanked by CDR or framework sequences together with a reverse primer downstream of the linker site (Gao and Paul, 1995; and Deng et al., 1995). The first round products are used in a second round of amplification with a forward primers upstream from a second restriction site. The resulting fragments are ligated into the wild type sFv at the appropriate restriction sites. These derivatives are then sequenced as previously described for the original sFv4 to confirm the location and identity of the substituted residues. The light chains of mAb41.4 which contains the important CDR 3 belongs to the immunoglobulin group III family and sequence comparisons and modeling is with the programs described by Kabat et al. (1992) and Bernstein et al. (1977).

Derivative sFv's with affinity for CREB generated by mutagenesis are screened by competitive ELISA on microtitre wells coated with recombinant CREB as previously described. These studies are used to generate a CREB specific sFv and allow for additional studies that discriminate between ATF and CREB activities. Competition will be with peptide F which represents the region of CREB analogous to that of ATF 1. Increasing concentrations of peptide are added to the solution containing sFv over a range from 0.01 μM to 1 μM and allowed to incubate. Detection of bound sFv is accomplished with the polyclonal goat anti-mouse Fab antibody and a peroxidase conjugated donkey anti-goat antibody. After addition of substrate the plate is read and results are plotted as percent inhibition of wells without competitor.

sFv derivative activity in epithelial and fibroblast cell lines (Hela, and 293T) is evaluated as described in Example 10. ATF1 is an abundant protein in continuously proliferating cell lines, such as HeLa, and in lymphoid tissues with high proliferative capacities (Masson et al., 1993). We will focus on changes in reporter gene expression in the epithelial and fibroblast cell lines following transfection with the sFv derivatives, hereafter referred to as sFv4atf for the improved ATF1 specific derivative and sFv4creb for the CREB specific derivative. The effect of the sFv on non-consensus CRE driven gene expression will be compared with that of consensus CRE driven promoters. For these studies we will use the strong, multiple CRE containing promoter from the CMV immediate early gene driving luciferase (pCMV-Luc) and the HTLV-I non-consensus TRE driving luciferase. The cloning of pCMV-luc has been previously described (Gilchrist et al., 1995). HeLa and 293T cells will be used because our previous studies investigating sFv4 activity were conducted in these cells, and the level of ATF1 and CREB are known. HeLa cells also support expression from the HTLV LTR. Fibroblasts may be studied for comparison purposes, with the use of 293T cells selected for high transfection efficiency. If differences of greater than 5 to 10 fold in the reduction of luciferase activity are observed further studies are possible with additional fibroblast cell lines to further investigate the issue of cell type contributing to overall promoter activity. Other epithelial cell types such as MCF-7, a mammary carcinoma cell may also be studied if differences of inhibitory effect based on cell type are observed.

Additional controls for activity of sFv include pAd ML-LUC and pRSV-LUC which do not contain CRE's or related TRE sequences in the promoters. Transfection protocols follow those described in Example 1 and results are standardized for transfection efficiency.

The above example for generation of improved sFv4 constructs is offered by way of illustration and the same or similar procedures can be applied in the generation of sFv against other transcription factors.

### Example 15

### Determination of Biologic Activity of sFvs in Cell Culture and Tumor Models

Effect of sFv expression on PCNA protein levels. PCNA is used as a biologic marker of sFv activity in transfected cells for several reasons, first it is an auxiliary protein for DNA polymerase delta (Mathews, 1989); second, two CRE's are located in its promoter and are critical for optimal expression (Huang and Prystowsky, 1996); third, mAb4 is capable of inhibiting PCNA promoter activity in vitro (Orten, et al., (1994)); fourth, protein levels of PCNA do not need to drop to below detectable levels to result in an effect upon cell replication (Feuerstein et al., 1995); and fifth, it is an abundant protein and can be detected in a semi-quantitative means by western blot and at the cellular level by immunohistochemistry (Feuerstein et al., 1995). Although this is a preferred marker, other means may be used in the practice of the invention, as recognized in the art. The concept of threshold effect, as demonstrated by PCNA is an important concept in developing a new therapeutic approach to cancer, since an important protein involved in cell proliferation does not need to be reduced to undetectable levels for an effect on cell replication to become apparent. It is desired to know whether partial but not complete interference with ATF1 and CREB function will lead to alteration in cell viability or proliferation rate.

Experiments utilize the sFv constructs in HeLa and 293 T cells for observing an effect on PCNA expression. These results establish a baseline for comparison with improved sFv's (Example 14). Two different promoters are utilized including the CMV IE and the EF promoter described previously. The CMV IE promoter resulted in the highest level expression in short term, transient transfections evaluated at 48 hours in 293T cells. Transfections are performed as previously described and both the CMV and the EF promoter driving sFv are used in addition to the control sFv targeted to VIP. Three different amounts of sFv, e.g. 5, 10 and 20 μg are used to detect a dose response effect. The total number of CRE's transfected at the different levels are controlled with the CMV-null construct. It is important to eliminate the possibility that decreased expression of any marker is due to something other than the system being saturated with CRE's which act to deplete the system of CRE binding proteins. In addition, transfection efficiency is controlled using β-gal constructs, and PCNA expression is normalized to the β-gal level. At least two approaches to measuring PCNA can be taken including western blot and immunohistochemistry. Total proteins are extracted at two time points following transfection of HeLa cells and 293T cells in 35 mm culture dishes. Constant amounts of protein are loaded into wells to allow comparison of pre and post transfection levels, and immunoblotted using a mouse monoclonal antibody against PCNA (Sigma). Actin can be probed following transfer to confirm that equal amounts of protein were loaded and allow for comparison and semi-quantitation. In recognition of the numerous parameters that influence expression as measured by western blot, separate transfected wells containing coverslips are utilized to perform individual cell analysis for expression of PCNA. A polyclonal anti-PCNA antibody and fluorescent labeled secondary antibody are used. β-gal is used to control for transfection efficiency. Protein expression is measured by fluorescent and standard illumination photography of coverslips. Results are evaluated to determine which time is optimal for demonstration of sFv effect on PCNA expression, compare effect of CRE and non-CRE containing promoters on sFv4 activity, and to establish a baseline for comparison with improved sFv's.

Determination of the effect of sFv on cell viability and proliferation rate in vitro is critical in the targeting of transcription factors for cancer therapy. Grim et al. (1996) showed that a sFv directed against erbB-2 decreased viability of lung carcinoma cells but not HeLa cells following transient transfection, presumably due to different levels of expression in the different cell lines. In the event transformants expressing the sFv do not remain viable an inducible sFv construct can be generated. Various parameters can be measured as indicators of sFv activity including cell viability and changes in doubling time or cell proliferation rate.

Cell viability following transfection is determined by dye exclusion and the MTS assay. The dye exclusion method is a simple way to obtain a general impression of the overall effect by using a vital dye in the cell culture dish at selected time points following transfection. Initially, a 48-hour and a 72-hour time point is selected for study with 5,10 and 20 μg of DNA. Total number of viable cells per high power field (20× power objective) are counted with an inverted microscope and comparison is made between results from four constructs. Constructs CMV-sFv4, the EF-sFv4, the CMV-null, and the CMV-VIP are used to control for effect of the additional introduced CRE's. Results are normalized for transfection efficiency. After conditions are optimized, the MTS assay is utilized to provide a more objective quantitation of activity. This assay utilizes the reduction of MTS (3,4,5 dimethyltiazol-2,5 diphenyl tetrazolium bromide) by mitochondrial dehydrogenase in viable cells for generation of a formazan product that can be measured spectrophotometrically. Separately, HeLa and 293T cells (2×10³ in 150 μl RPMI media plus 10% FBS) are added to each well of a 96 well plate and allowed to plate overnight. The following day the cells are transfected and held for either 48 or 72 hours. MTS is added and the plate is incubated for 2 h to form formazan crystals. After removal of the media and washing, dimethyl sulfoxide (200 μl) is added to each well and the plate hand-agitated. The O.D. is measured at 540 nm and results are compared for each of the constructs utilized as described above.

Effect on cell proliferation is studied in two ways using a proliferation rate (or doubling time) assay and cell cycle distribution (or proliferation index) as determined by flow cytometry. In addition to the ability to detect apoptotic cells, flow cytometry provides a reproducible measurement of effect on proliferation as measured by proliferation index (PI). The effect on doubling time is plotted by directly counting cells originally plated at a density of 1×10⁴ in six-well plates. Cells are examined every two days for 21 days and counted with a Coulter counter. The fraction of cells that are non-viable or non-staining are compared to controls. It is determined whether transfected cells are halted in a specific phase of the cell cycle.

For studies by flow cytometry, exponentially dividing cells are collected from each time point and resuspended at 2×10⁵/ml in Vindelov's reagent (TBS, ribonuclease A, propidium iodide, Nonidet p-40, for 1-2 h prior to analysis (Vindelov, 1977). Vindelov's reagent is used to create “bare nuclei” with minimal forward scatter signal. Cells are analyzed at a reduced flow rate (150 cells/sec.) and sorted according to their stage in the cell cycle, and proliferative index or apoptotic state is determined. Samples are analyzed by flow cytometry and the fractions of the cells in G1, S or G2-M phase are determined.

The parameters used are varied depending on the specific application. Additional parameters include maintenance or loss of contact inhibition, cell morphology, and anchorage independent growth as measured in soft agar. As an alternative measurement of cell proliferation, thymidine incorporation may be used to measure cell proliferation, in which case 0.5 μCi of ³H-thymidine is added to each well, incubation for 6 h followed by washing and recovery of cells with a cell harvester. Incorporation of isotope is determined by scintillation counting and comparisons made with controls.

Comparison of ATF1 and CREB protein levels in experimental cells. Although CREB is known to be ubiquitously expressed, considerable variation in the level of CREB expression among different cell lines has been observed. The actual level of CREB expression in Hela is considerably lower than that in other transformed cell lines, such as 293T (Masson et al., 1993). Cell type specific factors may contribute to the level of CREB expression or the levels may be completely independent of cell type. Therefore, the relative level of CREB and ATF1 in the cells being studied is determined before and after transfection. mAb41.4 is used to characterize the levels of ATF1 and CREB in aliquots of the cells taken at time of transfection and at 48 hrs following transfection and in stably transformed cells before and after release of sFv repression by doxycycline. Mab41.4 recognizes a common epitope in ATF1 and CREB which allows for the simultaneous comparison of expression of these two factors in cells. Extraction procedures and immunoblotting are performed as described by Orten et al. (1994). The immunoblot assay is able to provide a semi-quantitative assessment of the level of ATF1 and CREB in the cells; a 2-fold increase or reduction in protein level can be recognized. The level of ATF1 and CREB is not altered by the sFv with these expression vectors unless binding by the sFv leads to increased degradation by cellular processes.

It has been discovered that the sFvs of the present invention are capable of entering the nucleus. The subcellular localization of the sFv in the nucleus was unexpected. There have been no reports, to date, of sFvs entering the nucleus and blocking activity of transcription factors. Evaluation of the subcellular localization of the other inhibitory agents of the invention can made by including a nuclear localization sequence in the vector and determining the effect upon intracellular activity. As described in Example 10, intracellular expression of sFv is capable of significant reduction in CRE containing promoters. Nuclear targeting of an inhibitory agent can confirm and quantify that the inhibitory agent is capable of entering the nucleus and whether cytoplasmic expression of the agent also results in binding to nuclear factors before import. If an inhibitory agent is locating in part in the cytoplasm, as was expected from other work with antibody fragments, then it should be possible to increase the inhibitory effect through nuclear targeting. Subcellular localization of proteins plays an important role in their function and several important characteristics of nuclear localization sequences (NLS) have been identified (Dang and Lee, 1989). CREB, ATF1, PAX, FLI and EWS are nuclear proteins and are thought to be rapidly shuttled to the nucleus after synthesis. The disruption of transcription factors in different cellular compartments provides insight into how transcription factors may function with greatest efficiency and activity. It has been previously reported that sFvs are not processed like natural separate heavy and light chain proteins and do not contain sequences for cytoplasmic membrane localization and release. Although other NLS known in the art can be used (Dang and Lee, 1989), the prototypic NLS from the SV40 large T antigen (PKKKRKVE) is conventionally used because it is the best characterized NLS and is the most likely sequence to provide nuclear localization in each of the cell types of interest (Rihs et al., 1991; and Roberts et al., 1987). The NLS must be located on an exposed surface to function appropriately (Rihs et al., 1991), and therefore an oligonucleotide containing NLS is typically inserted in the pEBV-GRE5 vector immediately adjacent to the 5′ end of the interchain linker and upstream from the light chain coding sequence in sFv4 to generate pEBV-GRE5sFv4/nu.

The tumorigenicity of the CCS cell line in nude mice has been demonstrated and is highly reminiscent of the Clear Cell Sarcoma tumor in humans (Hiraga et al. 1997). For studies in mice, the sFv with high affinity for ATF1 or CREB, as discovered in Example 14, was used to demonstrate inhibition of tumorigenicity of cells in nude mice. It is then determined whether a stably transformed CCS cell remains viable and whether an inducible system is developed. Transfectoma experiments are conducted using a fixed number of treated or untreated cells (i.e. 10⁷) injected into the experimental mouse. Transfected cells are selected in G418 and integration of the sFv is confirmed by southern blot. Cells are collected during exponential growth phase and introduced into the thigh muscle or subcutaneously. To reduce the total number of animals used, only one promoter construct is selected and either of the sFv constructs that show high affinity or specificity for ATF and CREB as well as the control anti-VIP construct.

Inducible expression of the sFv's. Inducible expression systems have been described and each have limitations, therefore our choice is based on several specific objectives. In our studies, we are attempting to obtain tight control of sFv expression for generation of stable transformants. The goal of an inducible system is to regulate temporal activity of the gene, relevant in this case because expression of the sFv may act to limit natural proliferation of cells (Disruption of ATF1 as part of the EWS-ATF1 chimeric protein will lead to cell death if these proteins are essential to prevent apoptosis or maintain cell proliferation). We will use the tetracycline inducible system to obtain stable transformants for subsequent introduction into mice (Furth et al., 1994). This system uses the tetracycline-regulated transactivator protein (tTA, composed of the repressor of the tetracycline-resistance operon and the activating domain of herpes virus VP16) in conjunction with a second construct that incorporates the tet resistance operon and a strong promoter such as CMV. Either an “on” or “off” system is used.

Prior to studies in mice with the inducible system, functionality of the system in cell culture assays is confirmed. In addition, a separate well of cells is harvested for detection of sFv expression by western blot.

Stable transformants of 293T are generated with plasmid pGT21, and separately, each of three different sFv expressing plasmids, pTet-sFv4, pTet-sFv4a, and pTet-sFv4c in addition to the parental vector without the sFv insert. Cells are transfected and allowed to grow in non-selective media for 48 hours after which they are maintained in DMEM containing G418. Selected clones are expanded for detection of sFv expression and for further work in vivo. In stably transformed cells, expression of sFv is minimal or absent, and upon induction, cell viability is reduced or eliminated.

The above example for determining biological activity of sFv4 in cell culture and tumor models is offered by way of illustration and the same or similar procedures can be applied in the determination of activity of other sFvs. Alternative approaches to establishing an inducible system include the recently described ecdysone system, the glucocorticoid inducible system and the metalothionine inducible promoter system. Two potential problems are known with these latter systems; the toxicity of heavy metals, and the relatively high basal transcriptional activity of the promoter.

### Example 16

### Anti-ATF1 mAbs Inhibition of EWS/ATF1 Binding to a CRE In Vitro

EWS/ATF1 incorporates the carboxyl terminal region of ATF1 containing the epitopes of the two anti-ATF1 mAbs used in these studies. Although both mAb4 and mAb5 recognize epitopes adjacent to the DNA binding domain of ATF1, mAb4 interferes with DNA binding by ATF1 in EMSA, and mAb5 super-shifts ATF1 without disrupting its DNA binding activity. The contribution of EWS to the overall conformation of the chimeric protein is unknown. EWS/ATF1 and ATF1 were used in gel shift assays with radio labeled CRE DNA to evaluate the ability of mAb4 and mAb5 to bind EWS/ATF and determine the effect of mAb4 and mAb5 on complex formation. EWS/ATF1 binding to CRE DNA has been previously demonstrated (Li et al., 1998; Brown et al., 1995; and Fujimura et al., 1996), however it is not known whether CRE sequences are the primary target in cells or whether other related DNA sequences are capable of being bound (Orten et al., 1994; and Gilchrist et al., 1995). For these studies, a consensus CRE (TGACGTCA) as occurs in the somatostatin promoter was utilized. EWS/ATF1 was expressed in 293T cells rather than bacteria to control for possible effects of post-translational modification (Orten et al., 1994; and Gilchrist et al., 1995). The presence of mAb4 inhibited EWS/ATF1 complex formation was detected by reduced band intensity in EMSA, whereas mAb5 super-shifted the EWS/ATF1 complex. Each reaction mixture included 5 μg 293T-EWS/ATF1, 2 μg antibody, 4% glycerol and 0.1% gelatin and was incubated at 30° C. This effect on complex formation was similar to that of mAb4 on ATF1/CRE complexes and the super-shift of ATF1/CRE complexes by mAb5. The EWS-N Ab (SantaCruz), which recognizes the amino-terminal region of EWS was used to verify the identity of the EWS/ATF1 complex and this antibody was capable of producing a partial super-shift. As expected, EWS-N had no effect on ATF1/CRE complexes. Specificity of EWS/ATF1 for the CRE was demonstrated with the addition of 100 fold excess of unlabeled AP1 and CRE competitors. Competition with unlabeled CRE resulted in a loss of ATF1 complexes, whereas competition using AP1 did not diminish the intensity of the complex. Each reaction mixture contained 50 ng rATF1, 2 μg antibody, 4.0% glycerol and 0.1% gelatin. AP1 is useful as a control for specificity since it differs from a consensus CRE by only one G-C base pair at its center. Isotype matched control Abs had no effect on complex formation. These studies indicated that although the EWS domain is considerably larger than the deleted amino portion of ATF1, it did not interfere with binding of specific epitopes by either mAb4 or mAb5.

The EWS/ATF1 fusion protein is hypothesized to be the primary genetic event leading to CCS. However, the level of EWS/ATF1 expression in primary tumor tissue has not been demonstrated previously. Extracts from SU-CCS-1 cells, a primary CCS tumor, and a primary human fibroblast cell termed HHF, were immunoprecipitated and analyzed by western blotting. Efficiencies of protein extraction and immunoprecipitation were both shown to be greater than 95%. HHF cells were utilized to represent non-transformed control cells of mesenchymal origin. Recombinant ATF1 expressed in E. coli BL21 and EWS/ATF1 expressed in 293T cells were used as markers for the proteins of interest. The EWS-N Ab (Santa Cruz) which recognizes the amino-terminal region of EWS was again used to confirm identity of the presumed EWS/ATF1 band. Due to the (Gilchrist et al., 1995; Kirschmeier et al., 1988) translocation, only one normal ATF1 allele remains in SU-CCS-1 cells and the CCS tumor (Bridge et al., 1991). However, levels of ATF1 were similar to those of nontransformed HFF fibroblasts with two alleles. The EWS/ATF1 band was considerably darker in comparison with the endogenous ATF1 band in the SU-CCS-1 cell line and the CCS tumor. Densitometric analysis indicated that EWS/ATF1 levels were 3.0 fold greater than those of ATF1 in the SU-CCS-1 cell extract and 10.6 fold greater than ATF1 in the CCS tumor extract. As expected, EWS/ATF1 was not present in the control HHF cell extract.

### Example 17

### sFv4 Inhibition of CRE Reporter Expression in HeLa and SU-CCS-1 Cells

As was discovered in Examples 4 and 10, inhibition of specific complex formation in vitro by mAb4 was predictive of decreased reporter expression in transfected cells. Since EWS/ATF1 binding to a CRE was inhibited in vitro by mAb4, a similar effect on transactivation was expected in cells following transfection of sFv4. HeLa cells were chosen for their relatively higher level of ATF1 versus CREB expression and their well-documented history of CRE-reporter activation. Transient cotransfection assays of HeLa cells were performed using a CRE-luciferase (luc) reporter and constructs expressing sFv4 (pFv4) and EWS/ATF1 (pEWS/ATF1). The reporter construct incorporated the strong CMV immediate early gene promoter which contains 5 CRE sequences. To normalize results for variation in transfection efficiency between experiments, an internal RSV-β-gal control was included in the transfection system.

The number of promoter elements present in each transfection was held constant by the addition of equimolar amounts of parental vectors. Transfection of 5 μg pEWS/ATF1 per 10⁶ HeLa cells produced a 3.3 fold increase in CRE-luc expression and use of 10 μg pEWS/ATF1 per 10⁶ cells produced a 6.5 fold increase. Cotransfection of pFv4 (10 μg per 10⁶ cells) into this system reduced the observed 6.5 fold increase in reporter expression to less than 3 fold, thus suggesting that sFv4 was capable of inhibiting CRE activation by EWS/ATF1 in HeLa cells. The levels of CRE-reporter expression in response to EWS/ATF1 were similar to those previously described (Chothia et al., 1987; Chothia, 1989; and Fisher et al., 1994). Expression of EWS/ATF following transfection was confirmed using immunofluorescent labeled antibodies.

The SU-CCS-1 cell line was derived from a CCS tumor that expresses endogenous EWS/ATF1 (Epstein et al., 1984) and optimal transfection conditions were unknown. Therefore, a green fluorescent protein (GFP) expressing construct was used to determine the optimal transfection method and time course to be used. A higher level of transfection efficiency was achieved using the liposome mediated system than with calcium phosphate. Expression of CRE-luciferase reporter measured over a 24 to 96 hour time course demonstrated the peak level occurred at 72 hours. Therefore, to evaluate the effect of sFv4 on endogenous EWS/ATF1 activity, transient transfections of SU-CCS-1 cells were performed using the liposome mediated method and luciferase activity was measured at 72 hours with CRE-luc reporter and increasing amounts (2.5 to 10 μg per 10⁶ cells) of pFv4. Luciferase reporter activity decreased proportionately as increasing amounts of pFv4 were transfected into the SU-CCS-1 cells. Activity was reduced by 80% when 10 μg pFv4 per 10⁶ cells was used and 90% reduction was observed at higher concentrations of pFv4. Previously, we have observed that 10 μg of pFv4 per 10⁶ cells decreased reporter activity by only 20% in the non-EWS/ATF1 expressing HeLa cell line (Bosilevac, et al., 1998). Therefore, the significantly greater decrease in reporter activity in SU-CCS-1 cells was likely to be due to the inhibition of the strong EWS/ATF1 activator by sFv4 and not inhibition of endogenous ATF1 activity. However, since the decrease in CRE reporter activity was reversed by over-expression of ATF1, either possibility remained. 1 μg pATF1 cotransfected with 2.5 μg pFv4 per 10⁶ SU-CCS-1 cells restored luciferase expression to near baseline levels, indicating that ATF1 competed for sFv4 binding and allowed free EWS/ATF1 or endogenous factors to activate the CRE reporter. In HeLa cells, the small effect on reporter activity may be due to the presence of other strong activating proteins that regulate expression as well as regulatory elements other than CRE. Although in vitro assays may not accurately reflect all aspects important to transcriptional regulation, the level of inhibition by sFv4 was predictive of results when cell viability was determined.

### Example 18

### Expression of sFv4 in SU-CCS-1 Cells Leads to Loss of Viability and Apoptosis

sFv4 was delivered to a majority of SU-CCS-1 cells to determine whether the inhibition of EWS/ATF1 activity would affect cell viability. As discovered in Example 17, GFP constructs demonstrated that less than 10% of the SU-CCS-1 cells were transfected by the liposome mediated system. The ability of a Moloney sarcoma retrovirus system (SRαMStkneo) to transduce the SU-CCS-1 cells was examined (Takebe et al., 1988; Kirschmeier et al., 1988; and Muller et al., 1991). An SRα retrovirus capable of expressing GFP demonstrated a transduction efficiency of 80% or greater. Therefore, to attain widespread delivery of sFv4 to the SU-CCS-1 cells, the SRα retroviral system was utilized and modified to express sFv4. The cDNA of sFv4 was placed into the SRα-PN construct and used to produce infectious amphotropic retrovirus. SU-CCS-1 cells were transduced with 10⁴ cfu of either SRα retrovirus expressing sFv4 (SRα-Fv4), the parental SRα-PN with no insert or a mock media preparation that simulated the infection conditions (control). The SU-CCS-1 cells were visually inspected daily following treatment. Control cells showed no decrease in density, grew to confluence and showed no reduction in viability. Cells exposed to SRα-Fv4 demonstrated membrane blebbing and cell nuclear condensation beginning at day 3, and these changes subsequently became apparent throughout the population. By day 5, the cytotoxic effects reached maximum and cell density began to decrease substantially. At day 7, viable cells were sparse and examination under 100× magnification showed considerable cellular debris. The experiments were repeated on three occasions with similar results. Conversely, less than 1% of cells exposed to SRα-PN demonstrated focal cytotoxic effects apparent at day 5, characterized by a reduction in cell size and focal membrane blebbing. However, the remaining cells continued to grow and proliferate to day 10 with no progressive loss in viability.

To correlate the physical appearance of cells with an objective measurement, the percentage of viable cells was determined by two different methods; trypan blue dye exclusion and the MTS assay (CellTiter AQueous™, Promega) (Example 1). The cells transduced with SRα-Fv4 showed a pronounced decrease in viability as measured by trypan blue dye exclusion, beginning at day 2 which became prominent by day 5 with only one third of the cells remaining viable. Corresponding to our visual observations, only 10% of the SU-CCS-1 cells remained viable as determined by dye exclusion at day 10. Control SRα-PN infected cells and mock transduced cells had similar percentages of viable cells throughout the course of study. Since the levels of viability in the control cells was 60% rather than the expected 90-100%, we investigated the effect of cell harvesting procedures on overall viability when measured by the dye exclusion method. The impact of harvesting cells by scraping was examined by comparison of results with the MTS assay which requires minimal cell manipulation. The viability of SRα-Fv4 transduced cells declined to 60% on day 3 and continued to decrease to 30% at day 7 as determined by MTS assay. In comparison, both the mock transduced cells and those transduced by SRα-PN demonstrated similar results with the percentage of viable cells starting at 100% and decreasing to 60% at day 7. Since the results by both trypan-blue exclusion and MTS assay were similar and corresponded to the visual appearance, we concluded that the expression of sFv4 had a significant effect on SU-CCS-1 cell viability, and based on the morphologic appearance, postulated that cell death may be occurring through a process of apoptosis.

The process of SU-CCS-1 cell death could occur through either necrosis or apoptosis, or a combination of both mechanisms (Raffray et al., 1997; Kroemer et al., 1998). The visual observations described above suggested that apoptosis was occurring in the SRα-Fv4 infected cells. In order to confirm these observations, aliquots of SU-CCS-1 cells from the same time course as the viability study were stained with Telford reagent and submitted for DNA content analysis by flow cytometry. Differences between controls and SRα-Fv4 infected cells were apparent at day 3 and continued to increase throughout the remainder of the 10 day time course. Transfection by SRα-Fv4 resulted in 25% apoptosis at days 5 to 7 which increased to 33% on day 10. At similar time points of day 5 and 10, 15% (p<0.05) and 18% (p<0.00005) of the mock transduced cells were apoptotic, respectively, and 10% (p<0.005) and 22% (p<0.0005) of the SRα-PN transduced cells were apoptotic, respectively (FIG. 6). Although values for the measurements of apoptosis induced by SRα-Fv4 made by flow cytometry are significantly different, the processes of harvesting, centrifugation, washing and staining could contribute to cell damage and death. Therefore, to minimize the effect of processing on apoptosis, cells were also fixed to slides and analyzed by TUNEL (Gavrieli et al., 1992) (Example 1). SRα-Fv4, SRα-PN and control cells were analyzed at days 1, 3, 5 and 10. A progressive increase in both the number and intensity of TUNEL positive SU-CCS-1 cells following transduction by SRα-Fv4 was apparent beginning at day 3 and became extensive between day 5 and day 10. At day 10, 30% of cells were TUNEL positive. No intensely dark-staining nuclei were observed in the control preparations at day 1.

Since the intracellular expression of sFv4 could potentially induce cell death due to cross-reactivity with ATF1 or CREB, retroviral transduction experiments were performed in HeLa cells in which ATF1 and CREB are readily detectable. HeLa cells were transduced with 10⁴ cfu of SRα-Fv4, SRα-PN or a mock media (control) preparation and assayed by the MTS method (Example 1). Although transient effects were again seen at day 1, no significant differences in cell viability were observed between the sFv4 and control treated cells. The absence of any reduction in the percentage of cells remaining viable indicated that sFv4 is not toxic to HeLa cells and support the conclusion that apoptosis in SU-CCS-1 cells was due to specific targeting of the EWS/ATF1 fusion protein rather than the inhibition of other transcription factors.

### Example 19

### Anti-FLI sFv Inhibits DNA Binding by EWS/FLI

Modifications of the original approach described by Winter and Milstein (1991) are used for the cloning of sFv using reagents from a kit by Pharmacia. Recombinant protein is generated using the pET14b expression vector described in Example 1 containing the EWS/FLI1 cDNA clone (provided by Dr. Marc Ladanyi). Mice are immunized with full-length protein after purification on a DNA cellulose column. Following an intra-splenic boost, the mice are sacrificed and the spleens removed. Total RNA is extracted and heavy and light chain cDNA synthesized and cloned using primers contained in the kit following manufacturer's instructions. The phagmid vector, pCANTAB5, is used which contains an IPTG-inducible lac promoter, ampicillin resistance, a signal peptide sequence, a gene3 structural peptide sequence, an amber stop codon between the insert site and gene3, a c-myc tag and an insertion site compatible with Sfi and NotI restriction ends which are present on the amplified VH and VL sequences. The heavy and light antibody chain PCR products are ligated together with a flexible 15 amino acid linker (Gly4-Ser)₃ (Amersham) and subsequently ligated into the NotI and SfiI sites of the vector. The amber codon permits expression of V domains as p3 fusion proteins on the phage surface depending on host strain (TG1 cells recognize amber as GLU whereas HB2151 cells recognize amber as a stop codon). Infection by M13-K07 helper phage permits packaging of the recombinant phagemid into phage expressing antibody. Antigen reactive phage are enriched by solid phase panning against recombinant FLI1 bound to culture dishes. After repeated washing, TG-1 cells are added to the dish and individual plaques are recovered and screened by ELISA. Phages capable of binding FLI1 are used to infect HB2151 cells for generation of soluble sFv or the clone is selected for cloning into other vectors. Confirmation of correctly sized inserts is made by digestion and viewing of a 0.7 kb band.

Soluble Fv is produced and quantitated as described by Gao and Paul (Gao et al., 1995). E. coli HB2151 are grown to an A₆₀₀ of 0.6 induced with 0.4 mM IPTG and grown at 25° C. for 4 hours. Periplasm is extracted in a high salt lysate buffer, clarified and dialyzed. Typical yields are 0.5 to 2.5 mg/L of culture. Quantitation of sFv is done by performing slot blotting and staining with an anti c-myc-tag antibody and a conjugated anti-mouse antibody. A c-myc-peptide-1 (Oncogene Scientific) standard curve was generated and the signal of sFv lanes are determined from the curve. The crude periplasmic extract is further purified through isoelectric focusing.

The relative affinity of sFv's for FLI1 will be evaluated by competitive ELISA on microtitre wells coated with recombinant EWS-FLI1 and FLI1 as previously described (Pack et al., 1995). These studies are intended to identify a FLI1 specific sFv with the highest possible affinity for further evaluation in gel shift assay. The competitive ELISA has proved to be an efficient method for screening activity of a moderate number of clones (i.e. 50-100). Increasing concentrations of protein are introduced into the solution containing sFv over a range from 0.01 μM to 1 μM and added to microtitre wells with antigen fixed to the plastic. Detection of bound sFv is accomplished with the polyclonal goat anti-mouse Fab antibody and a peroxidase conjugated donkey anti-goat antibody. After addition of substrate the plate is read and results are plotted as percent inhibition of wells. Following the mapping of the epitope, competitive ELISA is again performed to confirm affinity using peptide epitope as competitor.

Mapping of the epitope is performed to determine whether it is located in the predicted region and then to fully define the minimum number of residues that are required to form the epitope. Recombinant FLI1 is generated and purified as described above. Thrombin and bromide cleavage sites have been identified that are predicted to generate fragments ranging from approximately 500 Dalton to 10 kDalton. Digested protein is electrophoresed and fragments are identified using either a 5′ anti-EWS antibody (N-EWS, SantaCruz Biotech) or anti-FLI sFv on western blots. Individual bands are submitted for peptide sequence analysis and following its localization, overlapping peptides of 15 to 20 residues are synthesized and used in competitive ELISA as described above.

The family and group to which the sFv heavy and light chain belong is first determined and then it is determined if the sequences are germline or contain alternations from germline. This is of long-term relevance as substitutions are evaluated for increase in affinity or to provide for other opportunities such as formation of diabodies. The sequence of the variable heavy and light chains of the sFv is determined by automated DNA sequencing and its protein sequence established. Using this data modeling of the CDR's with the commercial version of AbM v2.1 (Oxford Molecular Ltd) is performed using the Molecular Modeling Core facility in the Eppley Cancer Institute (see Example 13). The modeling program is used to consider possible substitution studies to investigate the effect of replacing or deleting amino acid residues predicted to play an important role in binding to antigen. The AbM program builds the most conserved regions of the V-domain (FRs) by comparison with the most homologous antibody structure in the Brookhaven databank, PDB (Martin et al., 1989; Chothia et al., 1987; and Chothia et al., 1989). After review of the predicted structure, key residues will be determined which are likely to be involved in direct contacts with epitope and model alanine substitutions to identify those residues predicted to have the greatest impact on binding. Of particular relevance for this study is the three dimensional structure of an mAb and synthetic peptide antigen of myohemerythrin (Stanfield et al., 1990). Since the specific epitope has been discovered, rapid recognition is possible of contacts between the CDR and peptide epitope.

The present invention has defined multiple strategies for generating and selecting derivatives of an sFv that show improvement in performance over the starting material (Tyutyulkova et al., 1994). Affinity improvement has been reported for an anti c-erbB-2 sFv in which a 2 to six fold reduction in the dissociation constant was obtained by so-called parsimonious mutagenesis (Schier et al., 1996). Parsimonious mutagenesis refers to the technique where oligonucleotides are designed to substitute at varying frequencies the parental amino acid residues. Using this approach it is possible to identify residues that; 1 play a role in structure, 2 modulate affinity, and 3, contribute to recognition. The screening of mutagenized sFv's may reveal those with increased affinity or a second round of mutagenesis can be pursued through which additional substitutions of the critical residues are generated. An alternative approach relies on knowledge of the key residues involved in binding, such as that reported by Riechmann and Weil (1993), who employed semirational design using site directed randomization of key residues followed by recloning and phage display. They mutagenized an anti 2-phenyloxazol-5-one (phOx) Fv after modeling the binding pocket. Using molecular modeling, residues predicted to be involved in antigen binding were identified. Degenerate oligonucleotides and PCR were used to substitute these residues with the resulting sFv's found to have a six fold improvement in affinity. A third approach, termed molecular affinity maturation can be used to improve the affinity of anticarbohydrate sFv's (Deng et al., 1995).

An sFv with anti-FLI1 activity is identified by gel shift assay (Example 16). EMSA results accurately predicted activity in cells although the actual level of inhibition could not be predicted and varied from cell type to cell type. A number of cellular promoters contain ETS-box sequences including c-fos, glycoprotein IIb (GpIIb), and the HTLV1-LTR. These first two ETS box containing sequences are used for probes by generation of 30 base pair oligonucleotides. Controls for these studies include cold oligonucleotide as competitors and the addition of unrelated cyclic AMP response element sequences (CRE) as a demonstration of specificity. sFv is added to reactions containing probe and EWS/FLI1 at three time points (5, 15, and 30 minutes) following equilibrium and then loaded onto acrylamide gels. Control sFv directed against ATF1 is used to demonstrate specificity of the antibody effect.

One of the powerful aspects of phage display cloning is that several clones with affinity for FLI1 are generated. Therefore if a first round of cloning does not identify the desired activity as determined by EMSA, the phage can be rescreened using full length FLI1 and then using subfragments of FLI1 as antigen bound to plastic microtitre wells. Alternatively, a panel of monoclonal antibodies can be generated (Example 1) and then sFv can be cloned from myeloma cell line cDNA. These studies will show that a target for generation of an inhibitory sFv can be selected and constructed through established procedures. Successful demonstration of this approach provides the information needed to target other fusion proteins associated with specific neoplasms in a similar manner.

### Example 20

### Characterization of Anti-FLI Activity in Cells

Cloning and expression of EWS/FLI and FLI. cDNA of EWS/FLI was obtained from Dr. Mark Ladanyi (Sloan Kettering Cancer Res. Inst.) and Ewing's Sarcoma cell lines and primary EWS and PNET cells were provided by Dr. Bridge (UNMC, Eppley Tissue Bank). The EWS/FLI coding sequence is removed from the pCDNA3.1 (−) (Invitrogen) vector as an approximately 1.6 KB fragment in preparation for its insertion into an expression vector pET14B (Novagen). Vectors were generated containing the full length EWS/FLI and also separately FLI. Mice are immunized with the full-length protein and the phage library is screened with FLI coated on plastic dishes as described in Example 19. Appropriate restriction sites were introduced by PCR in order to generate a protein that can be purified on DNA cellulose and then cleaved with a thrombin site amino to the EWS domain. This approach minimizes the interference of the HIS tag or other tags with the antibody raised against the protein.

Transient cotransfection assays of 293T cells is performed using an ETS box-luciferase (luc) reporter and constructs expressing sFv and EWS/FLI. The derivation of the EWS/FLI vector is described in Example 19. The reporter construct incorporates the ETS containing SRE promoter. To normalize results for variation in transfection efficiency between experiments, an internal RSV-α-gal control is included in the transfection system. The number of promoter elements present in each transfection is held constant by the addition of equimolar amounts of parental vectors. Transfections include up to 5 μg each of pEWS/FLI, psFv and pSRE-Luc per 10⁶ 293T cells. Luciferase activity is determined as previously described (Example 17) and results of three experiments are averaged. It is believed that the introduction of pFv4 into this system reduces the previously determined increase in reporter expression by more than 50%.

Localization of sFv protein in the nucleus is detected in 293T cells and also after sFv is introduced by retroviral vector. The detection of sFv requires a modification of the original clone (Example 19), in that an additional tag is introduced. This is because the myc tag did not allow detection of sFv directed against ATF1. Computer assisted modeling suggests that the myc tag does not extend sufficiently beyond the beta sheets of the framework regions to be detected and therefore a strept tag is added. The original strept tag generated by PCR for the purpose of using restriction sites in the pCANTAB vector is used. Following transfection into 293T cells, sFv (strept) is detected using commercial reagents. The cells are permeabilized and streptavidin is detected with a fluorescein labeled antibody and visualized under fluorescence microscopy. It has been discovered that the sFvs of the present invention are capable of entering the nucleus without the addition of a nuclear localization signal (NLS). This is further exemplified in Example 24.

Alternative methods for delivery of the sFv into the cell include placing the sFv expression cassette into the retroviral vector (Example 1). Specific effect of sFv directed against FLI upon ETS box containing promoters is demonstratable as compared to CRE containing or viral promoter elements. Alternatively, a wide variety of other promoters are available to evaluate activity or it is possible to synthesize variants of the ETS box to more fully investigate the range of effect. It is believed that sFv inhibits binding in a general manner and sterically interferes with more than one or two interactions between amino acid side chains and the nucleotide bases within the ETS box.

### Example 21

### Role of EWS/FLI in Maintenance of Cell Viability

Retroviral delivery systems. Retroviral transduction of an antisense cyclin G1 construct into osteosarcoma cells has been shown to inhibit tumor growth in vivo (Chen et al., 1997). The SRαMStkneo retrovirus system is an alternative which was used because of the advantages it provides for infection of mesenchymal cells. The SRa promoter was derived from the SV40 early promoter and the U5 region of HTLV-1 (not including the CREs in U3) which achieves high-level gene expression and combined with the Moloney murine sarcoma virus (Kelly et al., 1998). To increase transduction, the vesicular stomatitis virus (VSV) envelope is inserted into the envelope of Moloney Sarcoma virus (Example 18). The VSV G proteins improve stability of envelope and allow for centrifugation of the free virus. A second improvement in generation of high titer virus comes from the use of the HIV LTR. Using this system, viral titers of 10⁹ have been achieved. Low titers in packaging cell lines have been addressed through concentration of viral supernatants to increase titer, and the VSV envelope protein described above. Unlike bone marrow progenitors or epithelial cells where the goal is to achieve infection in a differentiated cell with low proliferative index, the EWS cells specifically and sarcomas in general are highly proliferative and are readily infected by retrovirus. Tumor cells are capable of retrovirus replication in transgenic mice (Koike et al., 1989). Alternative vectors include adenovirus, lentivirus, or Herpes tk vectors (Robbins et al., 1998).

Infection of cells is performed using 3 ml of retroviral stock per well in a 6 well plate in the presence of 4 mg/ml hexadimethrine bromide (polybrene). Plates containing cells are spun in at 1250×g in a refrigerated centrifuge at 18° C. EW/PNET-1 cells are infected with 10⁴cfu of either the retrovirus expressing sFv (SRαFv), a parent retroviral vector with no insert (SRα-PN), or a mock media preparation that simulates the infection conditions without retrovirus (control). The retroviral titer is determined by colony forming assay in 3Y1 cells grown in MEM containing 5% bovine calf serum (BCS) and 800 mM G418 (Geneticin). High titer retrovirus is produced by centrifugation of viral culture supernatant from Y1 cells grown in 850 cm roller bottles at a density of 8 to 9×10⁶ cells per roller bottle. Vector supernatants are centrifuged at 8500 rpm at 4° C. for 18 hr. The vector pellets are resuspended in 0.5 Ultradoma-PF and 1 ml aliquots will be stored at −70° C. Representative aliquots are thawed for determination of titer.

Three different cell strains have been characterized that express the EWS/FLI fusion protein in addition to SK-ES-1. (The term cell strain is used to indicate that the cells have not exceeded 50 passages from their original derivation.) These experiments are used to demonstrate that the chimeric protein EWS-FLI1 plays a key role in induction and maintenance of the neoplastic phenotype, and that disruption of EWS-FLI1 through intracellular expression of sFv is toxic to Ewing's sarcoma tumor cells. EWS cell lines were selected which were originally derived from a Ewing's Sarcoma tumor and which closely resemble the primary human tumor in regards to the level of expression of EWS-FLI1. All observations are made in duplicate six well plats.

The breadth of activity of the sFv is examined by determining levels of specific proteins in the Ewing's Sarcoma cell line, SK-ES-1, in order to determine whether levels of EWS-FLI1 remain constant following introduction of sFv (implying a toxic effect from transient loss of EWS-FLI1 activity) or that levels are reduced. If changes in protein level are seen, further investigation such as whether binding of EWS-FLI1 by an sFv leads to increased protein degradation through ubiquination or other pathways is undertaken. In addition to EWS-FLI1, levels of Bcl-2, and FLI1 are followed by western blotting. Bcl-2 is a commonly studied protein due to its key role as an inhibitor of apoptosis. The proteins selected for study are relatively abundant in cells and levels can be monitored semi-quantitatively by western blot and at the cellular level by immunohistochemistry. Total proteins are extracted at two time points following infection by SRα sFv in 35 mm culture dishes. Constant amounts of protein are loaded into wells to allow comparison of pre and post transfection levels, and immunoblotted using mouse monoclonal antibody against EWS-FLI1, Bcl-2 and FLI1. B-actin is probed following transfer to confirm that equal amounts of protein were loaded and allow for comparison and semi-quantitation. Commercial antibodies are used to characterize the levels of FLI1 in aliquots of the cells taken at time of transfection and at 48 hrs following transfection. Extraction procedures and immunoblotting are performed as described in Example 1.

The viability of EWS cells following infection by SRα-Fv or control SRα-PN is determined by the trypan blue dye exclusion method and the MTS assay (Example 1). Cytopathic effect is also monitored. Both methods are used as a simple way to obtain a general impression of the overall effect by using selected time points following transduction. 48-hour and 72-hour time points are initially used with 5,10 and 20 μg of DNA. EWS cells are plated at 2×10⁴ cells/well and infected with SRα-Fv or controls (0.2 ml/well). Total number of viable cells per high power field (20× power objective) with an inverted microscope are counted and comparison made between results from control constructs and sFv. For the dye exclusion assay, cells are counted directly with a hemocytometer as described. Grids are counted, quantitating blue cells and white cells, until a total of at least 400 cells is reached. It is believed that the baseline level of viability using control retrovirus ranges between 80 and 90%, and that less than 20% of cells infected by SRα Fv remain viable at day 10 post infection.

The MTS assay is performed as described in Example 1. The absorbance readings of three experiments are normalized to one another and the results plotted as percent viable cells versus time.

Several morphologic changes are apparent in cells that may suggest an underlying process leading to cell death. These include the presence of apoptotic bodies or pyknosis and cell shrinkage as opposed to cell swelling. Cultured cells are reviewed by light microscopy for such features described in the literature, however electron microscopy remains the reference standard for differentiating between necrosis and apoptosis.

### Example 22

### Anti-PAX sFv inhibits DNA binding by PAX/FHKR

Cloning and expression of PAX/FHKR and PAX. A PAX/FHKR cDNA clone is generated from a full length fragment by PCR from ARM cell lines (TTC-487 and SJRH30) cDNA and cloned in a TA vector. The cell lines TTC-487 and SJRH30 are A-RMS cell lines that are PAX-FKHR positive (provided by Dr. Julia Bridge, UNMC, Eppley Tissue Bank). After generation of full length PAX/FKHR cDNA and cloning in TA vector, the PAX/FKHR coding sequence is removed and placed in the pcDNA3.1 (−) (Invitrogen) vector and into an expression vector, for example pET14B (Novagen). Appropriate restriction sites are introduced by PCR as necessary with the goal of generating both the PAX and PAX/FKHR proteins that are purified by affinity chromatography using the HIS tag. Purified proteins are used for coating of microtitre wells in the screening assay and for immunization of mice (Example 1).

Generation and screening of an anti-PAX sFv is performed as described in Example 19, except as noted. Recombinant protein is generated using the pET14b expression vector described in Example 1 containing the PAX/FHKR cDNA clone (provided by Dr. Rousell) or through cDNA cloning (Example 1). Antigen reactive phage are enriched by solid phase panning against recombinant PAX bound to culture dishes.

Screening of the sFv's for anti-PAX binding activity is performed by EMSA and soluble Fv is produced and quantitated as described in Example 19. Recombinant PAX is generated and purified as described above and the epitope mapped (Example 19). Digested protein is electrophoresed and fragments identified using either PAX IgG antibody (Santa Cruz Biotech) or anti-PAX scFv on western blots.

Sequencing of the sFv and the generation and selection of ScFv derivatives is performed as described for EWS/FLI1 (Example 19).

### Example 23

### Characterization of Anti-PAX Activity In Vitro and in Cells

Effect of sFv on PAX/FHKR binding to DNA is investigated as described for EWS/FLI1 (Example 19) using probe and PAX/FKHR.

Transient cotransfection assays of 293T cells are performed generally as described for EWS/FLI1 (Example 20) using the PAX/FKHR vector as described above. The reporter construct incorporates the homeodomain promoter region (pHD-luc)(provided by Dr. Rousel). Transfections include up to 5 μg each of pPAX/FHKR, psFv and pHD-Luc per 10⁶ 293T cells. It is believed that the introduction of pFv4 into this system reduces the previously determined increase in reporter expression by more than 50%.

Co-localization of sFv and PAX/FHKR is investigated as described for EWS/FLI1 (Example 20). Subcellular localization of sFvs are determined as described for EWS/FLI1 sFvs (Example 20). FKHR and PAX are also nuclear proteins and are thought to be rapidly shuttled to the nucleus after synthesis.

### Example 24

### Nuclear Localization of GFP/Fv4

Three plausible explanations for the effect of sFv4 on EWS/ATF1 in SU-CCS-1 cells were considered. sFv4 could bind EWS/ATF1 in the nucleus to prevent its subsequent binding to DNA in a steric or allosteric manner, or sFv4 could bind EWS/ATF1 in the cytoplasm leading to its immunodepletion or premature degradation. Alternatively, sFv4 may enter the nucleus already bound to EWS/ATF1. It has been discovered that sFv4 localizes to the nucleus and binds EWS/ATF1.

A GFP/Fv4 construct was generated by fusing green fluorescent protein (GFP) to sFv4. The chimeric GFP/Fv4 protein was purified by affinity chromatography and electrophoretic mobility shift assay demonstrated that it retained inhibitory activity. COS1, HeLa and SU-CCS-1 cells were transfected with either pCMV-GFP/Fv4 or pEGFP. After 24 hr. incubation, the cells were observed under a fluorescent microscope, and subcellular localization of GFP/Fv4 and GFP was recorded. Nuclear localization of sFv4 was confirmed by immunohistochemical staining for His6 and c-myc peptide tags. Fluorescent microscopy demonstrated that GFP/Fv4 localized to the nucleus while GFP alone is diffusely present throughout the cell.

293T cells were transfected with either pCMV-Fv4 or pCMV-GFP/Fv4. After 24 hr. incubation, cytoplasmic and nuclear extracts were prepared. A slot blot was performed and analyzed by a fluorimeter. Fluorimetric analysis of cytoplasmic and nuclear extracts on slot blot confirmed these localizations. The V_{H} chain of sFv4 was sequenced by the Eppley Core Facility. Framework (Fw) and Complement Determining Regions (CDR) were determined. Molecular modeling of the sFv4 V_{H} chain was performed by The Swiss Protein Modeling™ program and visually examined with Swiss View™ software. Modeling revealed a patch of basic residues indicating a discontinuous nuclear localization sequence.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### LIST OF REFERENCES

Ackers, G. K. (1973). “Studies of protein ligand binding by gel permeation techniques.” Methods of Enzymology 27:441-55.
Anand, N. N., et al. (1991). “Bacterial expression and secretion of various single-chain Fv genes encoding proteins specific for a Salmonella serotype B O-antigen.” J. of Biological Chemistry 266(32):21874-21879.
Anderson, M. G. and W. S. Dynan (1994). “Quantitative studies of the effect of HTLV-1 Tax protein on CREB protein DNA binding.” Nucleic Acids Research 22(15):3194-3201.
Anderson, W. F. (1998). “Human gene therapy.” Nature 392:25-30.
Arber, N., et al. (1997). “Antisense to cyclin D1 inhibits the growth and tumorigenicity of human colon cancer cells.” Cancer Res 57:1569-1574.
Arevalo, J. H., et al. (1993). “Molecular basis of cross-reactivity and the limits of antibody-antigen recognition.” Science 365:859-863.
Armstrong et al. (1992). Proc. Natl. Acad. Sci. USA 90:7303-07.
Ausubel, F. M., et al. (1992). Current Protocols in Molecular Biology, Green-Wiley, Brooklyn, N.Y.
Baranger, A. et al., (1995).
Barr, F. (1997). Current Top Microbiol. 220:113-129.
Barton, K., et al. (1996). “Defective thymocyte proliferation and IL-2 production in transgenic mice expressing a dominant-negative form of CREB.” Nature 379:81-85.
Beaucage and Caruthers (1981). Tetrahedron Letters 22:1859-1862.
Bernasconi, M., et al. (1996). “Induction of apoptosis in rhabdomyosarcoma cells through down-regulation of PAX proteins.” Proc. Natl. Acad. Sci. 93:13164-13169.
Bernstein, F. C., et al. (1977). “The protein data bank. A computer based archival file for macromolecular structures.” J. Molecular Biology 112:535-542.
Bertolotti, A., et al. (1998). Mol. Cell. Biol. 18(3):1489-1497
Bills, N. D., et al. (1992). “Delayed tumor onset in transgenic mice fed a low-folate diet.” J. Nat. Cancer Inst. 84:332-7.
Bird, et al. (1988). Science 242: 423-26.
Blendy, J. A., et al. (1996). “Targeting of the CREB gene leads to up-regulation of a novel CREB mRNA isoform.” EMBO Journal 15(3):101-109.
Boehm, J., et al. (1998). J. Biol. Chem. 273(12):7169-7176.
Bose, S., et al. (1997). “Guidelines in selecting ligand concentrations for the determination of binding constants by affinity capillary electrophoresis.” J. Chromatog B (in press).
Bosilevac, J. M., et al. (1998). “Inhibition of activating transcription factor 1- and cAMP-responsive element-binding protein-activated transcription by an intracellular single chain Fv fragment.” J. Biol. Chem. 273(27):16874-9.
Bosilevac, J. M., et al., J. Biol. Chem. (in press).
Bridge, J., et al. (1990). “Chromosomal abnormalities in clear cell sarcoma.” Am. J. Clin, Pathol. 93:26-31.
Bridge, J., et al. (1991). “Cytogenetic findings in clear cell sarcoma of tendons and aponeuroses.” Cancer Genet. Cytogenet. 52:101-106.
Brown, A. D., et al. (1995). Oncogene 10(9):1749-1756.
Brown, A. D., et al. (1995). “Promoters containing ATF-binding sites are de-regulated in cells that express the EWS/ATF1 oncogene.” Oncogene 10:1749-1756.
Bruccoleri, R. E., et al. (1988). “Structure of antibody hypervariable loops reproduced by a conformational search algorithm.” Nature 335:564-568.
Bruccoleri, R. E. (1993). “Application of systematic conformational search to protein modeling.” Molecular Stimulation 10:151-174.
Buchner, J., et al. (1989). “Alternatively folded states of an immunoglobulin.” Biochemistry 30:6922-6929.
Busch, S. J., et al. (1990). “Fos, Jun and CREB basic-domain peptides have intrinsic DNA-binding activity enhanced by a novel stabilizing factor.” Oncogene 5:1549-56.
Cantor, C. R., et al. (1980). “Forster theory of energy transfer.” Biophysical Chemistry Part II: Techniques for the Study of Biological Structure and Function 451:54. W.H. Freeman and Co., Oxford, Eng.
Chalepakis, G., et al. (1991). “The molecular basis of the undulated/Pax1 mutation.” Cell 66:873-884.
Chen, D. S., et al. (1997). “Retroviral vector-mediated transfer of an antisense cyclin G1 construct inhibits osteosarcoma tumor growth in nude mice.” Hum. Gene. Ther. 8(14): 1667-1674.
Cho, Y., et al. (194). “Crystal structure of a p53 tumor suppressor-DNA complex: Understanding tumorigenic mutations.” Science 265:346-355.
Chothia, C., et al. (1987). “Canonical structures for the hypervariable regions of immunoglobulins.” J. Mol. Biol. 196(4):901-917
Chothia, C., et al. (1989). “Canonical structures for the hypervariable regions of immunoglobulins.” Nature 342(6252):877-883.
Chothia, C., et al. (1989). “Conformations of immunoglobulin hypervariable regions.” Nature 342:877-883.
Churchill, M. E. A., et al. (1994). “Crystal structure of a peptide complex of anti-influenza peptide antibody FAb 26/9: Comparison of two different antibodies bound to the same peptide antigen.” J. Mol. Biol. 241:534-556.
Dang, C. V., et al. (1989). “Nuclear and nucleolar targeting sequences of c-erb-A, c-myb, N-myc, p53, HSP70, and HIV tat proteins.” J. Biol. Chem. 264(30): 18019-18023.
Darsley, M. J., et al. (1985). “An approach to the study of anti-protein antibody combining sites. In: Investigation and exploitation of antibody combining sites.” Plenum Press, 63-68
de la Paz, et al. (1986). EMBO. J. 5(2):415-425.
Deloulme, J., et al. (1997). J. Biol. Chem. 272(43):27369-27377.
Deng, S. J., et al. (1995). “Synthetic antibody gene libraries for in vitro affinity maturation.” Methods Mol. Biol. 51:329-342.
Desplanque, D., et al. (1994). “Multimerization behaviour of single chain Fv variants for the tumour-binding antibody B72.3.” Protein Eng. 7(8):1027-1033.
Dignam et al. (1983). Nucleic Acids Res. 11:1475-1489.
Duan, L., Z., et al. (1995). Hum. Gene Ther. 6:1561-1573.
Ellenberger, T. E., et al. (1992). “The GCN4 basic region leucine zipper binds DNA as a dimer of uninterrupted a-helices: Crystal structure of the protein-DNA complex.” Cell 71:1223-1227.
Ellis, M. J. C., et al. (1995). “Activating transcription factor-1 is a specific antagonist of the cyclic adenosine 3′,5′-monophosphate (cAMP) response element-binding protein-1-mediated response to cAMP.” Molecular Endocrinology 9:255-265.
Epstein, A., et al. (1984) Cancer Res. 44:1265-1274.
Erickson, J. et al. (1990). Science 219:527-533.
Farnham, P. J. and R. T. Schimke (1986). Mol. Cell. Biol. 6:23922401.
Feuerstein, N., et al. (1995). “Rapamycin selectively blocks IL-2 induced PCNA gene expression in T lymphocytes: Evidence for inhibition of CREB/ATF binding activities.” J. Biol. Chem. 270:94549458.
Fine, R. M., et al. (1986). “Predicting antibody hypervariable loop conformations II: Minimization and molecular dynamics studies of McPC603 from many randomly generated loop conformations.” Proteins: Struct. Funct. Genet. 1:342-362.
Fisher, D. E., et al. (1993). “High affinity DNA-binding myc analogs: Recognition by an a helix.” Cell 72:467-476.
Fisher, R. J., et al. (1994). “Surface plasmon resonance based methods for measuring the kinetics and binding affinities of biomolecular interactions.” Curr. Opin. Biotechnol. 5:389-395.
Foulkes, N., et al. (1996). Oncogene 10(9):1749-1756.
Fredericks, W., et al. (1995). “The Pax3-FKHR fusion protein created by the t(2;13) translocations in alveolar rhabdomyosarcoma is a more potent transcriptional activator than Pax3.” Mol. Cell Biol. 15:1522-1535.
Friguet et al. (1985). J. Immunol. Methods 77:605-319.
Fujimura, Y., (1996). “The EWS-ATF-1 gene involved in malignant melanoma of soft parts with t(12;22) chromosome translocation, encodes a constitutive transcriptional activator.” Oncogene 12:159-67.
Furth, P. A., et al. (1994). “Temporal control of gene expression in transgenic mice by a tetracycline-responsive promoter.” Proc. Natl. Acad. Sci. 91:9302-9306.
Galien, R., et al. (1994). “Differential effects of c-jun and CREB on c-AMP response element activation by Ha-ras.” Oncogene 9:1101-1108.
Gao, Q. S., et al. (1995). “Site-Directed Mutagenesis of Antibody-Variable Regions.” In Methods in Molecular Biology, Vol 51: Antibody Engineering Protocols. Edited by S Paul Humana Press Inc., Totowa, N.J. Chap. 21 pp. 319-327; 1995
Gao, Q. S., et al. (1994). “Molecular cloning of a proteolytic antibody light chain.” J. of Biological Chemistry 269(51):32389-32393.
Gao, Q. S., et al. (1995). “Site-directed mutagenesis of proteollytic antibody light chain.” J. Mol. Biol. 253:658—664.
Gao, Q. S., et al. (1995). “Molecular cloning of a proteolytic antibody light chain.” J. Biol. Chem. 270:20870.
Gavrieli, Y., et al. (1992). J. Cell. Biol. 119:493-501.
Gilchrist, C. A., et al. (1995). “Transcriptional inhibition by a monoclonal antibody Fab fragment.” Antibody, Immunoconjugates and Radiopharmaceuticals 8(4):281-298.
Gilchrist, C. A., et al. (1996). “Evidence for the role of cyclic AMP-responsive elements in human virus replication and disease.” Journal of Biomedical Science 3(5):96-127.
Glockshuber, R., et al. (1992). “The disulfide bonds in antibody variable domains: effects on stability, folding in vitro, and functional expression in Escherichia coli.” Biochemistry 31:1270-1279.
Gonzalez, G. A., et al. (1989). “Cyclic AMP stimulates somatostatin gene transcription by phosphorylation of CREB at serine 133.” Cell 59:675-680.
Goss, D. J., et al. (1982). “Kinetic studies on the interaction of chain initiation Factor 3 with 70S Escherichia coli ribosomes and subunits.” J. Biol. Chem. 257:10119-10127.
Graus-Porta, D., et al. (1995). Mol. Cell Biol. 15:1182-1191.
Griffiths, A., et al. (1993). EMBO. J. 12:725-734.
Grim, J., et al. (1996). “erbB02 knockout employing an intracellular single-chain antibody (sFv) accomplishes specific toxicity in erbB-2 expressing lung cancer cells.” Am. J. Respir. Cell Mol. Biol. 15(3):348-354.
Hage, D. S., et al. (1997). “Recent advances I chromatographic and electrophoretic methods for the study of drug protein interactions.” Anal. Chem. (in press).
Hai, T. et. al. (1989). Genes and Dev. 3:2083-90.
Hai, T. W., et al. (1988). “Analysis of the role of the transcription factor ATF in the assembly of a functional pre-initiation complex.” Cell 54:1043-1051.
Hileman, R. E., et al. (1994). Bioconjugate Chemistry 5:436-444.
Hinrichs, S. H., et al. (1991). “Myxoid Chondrosarcoma with a 12;22 translocation.” Cancer Genet. Cytogenet.______
Hiraga, H., et al. (1997). “Establishment of a new continuous clear cell sarcoma cell line. Morphological and cytogenetic characterization and detection of chimaeric EWS/ATF-1 transcripts.” Virchows Arch. 431:45-51.
Hodgeson, J. (1991). Biotechnology 9:19-21.
Holliger, P., et al. (1993). “‘Diabodies’: Small bivalent and bispecific antibody fragments.” Proc. Natl. Acad. Sci. USA 90:6444-6448.
Hsuch, Y. P., et al. (1995). “Overexpression of activation transcriptional factor 1 in lymphomas and in activated lymphocytes.” J. of Immunology 154:5675-5683.
Huang, D., et al. (1994). “Promoter activity of the proliferating-cell nuclear antigen gene is associated with inducible CRE-binding proteins in interleukin 2-stimulated T lymphocytes.” Mol. Cell. Biol. 14:4233-4243.
Huang, D. Y., et al. (1996). “Identification of an essential cis-element near the transcription start site for transcriptional activation of the proliferating cell nuclear antigen gene.” J. Biol. Chem. 271:1218-1225; 1996
Hurst et al. (1991). Nucleic Acids Res. 19:4601-4609.
Jameson, D. M., et al. (1980). “Fluorescence anisotropy applied to biomolecular interactions.” Methods in Enzymology 246:283-300.
Jean, D., et al. (1998). Journal of Biological Chemistry 273(38):24884-24890.
Kabat, E. A., et al. (1992). “Sequences of proteins of immunological interest.” U.S.A.: U.S. Department of Health and Human Services (Edition 5).
Kelly, D., et al. (1998). “Transcriptional Activation of the Type II Transforming Growth Factor-beta Receptor Gene upon Differentiation of Embryonal Carcinoma Cells.” J. Biol. Chem. 273(33):21115-24.
Kirschmeier, P., et al. (1988). DNA 7(3):219-225.
Kohler, G. and Milstein, C. (1976). Eur. J. Immunol. 6:511-519.
Koike, K., et al. (1989). “Development of gastric carcinoma in transgenic mice expressing adenovirus.” Ela. and Elb. Proc. Natl. Acad. Sci. 86:5615-19.
Konig, P., et al. (1993). “The X-ray structure of the GCN4-bZip bound to ATF/CREB site DNA shows the complex depends on DNA flexibility.” J. Mol. Biol. 233:139-154.
Kroemer, G., et al. (1998). Ann. Rev. Physiol. 60:619-642.
Kubota, S., et al. (1996). “Nuclear preservation and cytoplasmic degradation of human immunodeficiency virus type 1 Rev protein.” J. Virol. 70:1282-1287.
Ladanyi, M. (1995). “The emerging molecular genetics of sarcoma translocations.” Diagn. Molec. Path. 4:162-173.
Ladbury, J. E., et al. (1995). “Measurement of the binding of tyrosyl phosphopeptides to SH2 domains: A reappraisal.” Proc. Natl. Acad. Sci. USA 92:3199-3203.
Lakowicz, J. R. (1983). “Energy Transfer Principles of Fluorescence Spectroscopy,” 303-339 Plenum Press, New York, N.Y.; 1983
Lam, P., et al. (1999). The oncogenic potential of the Pax3-FKHR fusion protein requires the Pax3 homeodomain recognition helix but not the Pax3 paired-box DNA binding domain.” Mol. Cell. Biol. 19:594-601.7
Levy-Mintz, P., et al. (1996). “Intracellular expression of single-chain variable fragments to inhibit early stages of the viral life cycle by targeting human immunodeficiency virus type 1 integrase.” J. Virol. 70(12):8821-8832.
Li, F., and Lee, K. (1998). Oncogene 16:1325-1331
Lin, L. N., et al. (1991). “Calorimetric studies of the binding of ferric ions to ovotransferrin and interactions between binding sites.” Biochemistry 30:11660-11669.
Lipton, S. A. (1997). “Janus faces of NF-kB: neurodestruction versus neuroprotection.” Nature Med. 3:20-22.
Look, T. L. (1997). Science 278:1059-64.
Luo, D., et al. (1995). “V1-linker-Vh orientation-dependent expression of single chain Fv containing an engineered disulfide-stabilized bond in the framework regions.” J. Biochem. 118:825-831.
Magnaghi, L, et al. (1996). “SRE elements are binding sites for the fusion protein EWS-FLI-1.” Nucleic Acids Res. 24:1052-1058.
Maguire, H. F., et al. (1991). Science 252:842-844.
Mainhart, C. R., et al. (1984). “A refined model for the variable domains (F_{V}) of the J539 b(1,6)-D-galactan-binding immunoglobulin.” Molecular Immunology 21:469-478.
Mantovani, R., et al. (1992). “Monoclonal antibodies to NF-Y define its function in MHC class II and albumin gene transcription.” EMBO J. 11:3316-3322.
Marasco, W. (1995). Immunotechnology 1:1-19.
Mariani, M., et al. (1991). “A new enzymatic method to obtain high-yield F(ab)₂ suitable for clinical use from mouse IgG1.” Molecular Immunology 28:69-77.
Martin, A. C. R., et al. (1989). “Modeling antibody hypervariable loops: A combined algorithm.” Proc. Nat'l. Acad. Sci. USA 86:9268-9272.
Masihi, K. N., et al. (1989). Int. J. Immuno Pharmacol. 8:339-345.
Masson, N., et al. (1993). “Identification of proteins that interact with CREB during differentiation of F9 embryonal carcinoma cells” [corrected and republished with original paging, article originally printed in Nucleic Acids Res 21(5): 1163-9]. Nucleic Acids Res 21:1163-1169; 1993
Masson, N., et al. (1993). “In vitro phosphorylation studies of a conserved region of the transcription factor ATF1.” Nucleic Acids Res. 21:4166-173.
Matsudaira, P. (1987). J. Biol. Chem. 262:10035-38.
Matthews, M. B. (1989). “The proliferating cell nuclear antigen, PCNA, a cell growth-regulated DNA replication factor. In Growth control during cell aging.” E. Wang and H. R. Warner, eds. CRC Press, Inc. Boca Raton, Fla., p. 89.
May, W., et al. (1993). “Ewing sarcoma 11;22 translocation produces a chimeric transcription factor that requires the DNA-binding domain encoded by FLI1 for transformation.” Proc. Natl. Acad. Sci. 90:5752-5756.
McCaffrey, P. G., et al. (1993). “Isolation of the cyclosporin-sensitive T-cell transcription factor NFATp.” Science 262:750-754.
McPherson, A. (1982). “Preparation and Analysis of Protein Crystals.” In: Sons, J. W., ed. Comparison of Two Fab-peptide Complexes. New York, N.Y., John Wiley and Sons.
Metallo, S. J., et al. (1997). “Certain bZIP peptides bind DNA sequentially as monomers and dimerize on the DNA.” (letter) Nat. Struct. Biol. 4:115-117.
Meyer, T., et al. (1993). Endocinology 132(2):770-80.
Meyer, T. E., et al. (1993). “Cyclic adenosine 3′,5′-monophosphate response element binding protein (CREB) and related transcription-activating deoxyribonucleic acid-binding proteins.” Endocrine Rev. 14:269-290.
Miranker, A., et al. (1991). “Functionality maps of binding sites: A multicopy simultaneous search method.” Proteins: Struct. Func. Genet. 11:29-34.
Montminy et al.(1986). Proc. Natl. Acad. Sci., USA 83: 6682-86.
Muller, A. J., et al. (1991). Mol. Cell. Biol. 11:1785-1792.
O'Shannessy, D. J., et al. (1993). “Determination of rate and equilibrium binding constants for macromolecular interactions using surface plasmon resonance: Use of nonlinear least squares methods.” 212:457-468.
O'Shannessy, D. J. (1994). “Determination of kinetic rate and equilibrium binding constants for macromolecular interactions: A critique of the surface plasmon resonance literature.” Curr. Opin. Biotechnol. 5:65-71.
Ohno, T., et al. (1994). Oncogene 9(10):3087-3097.
Ohtani, F. N., (1994). “A silencer element in the retinoblastoma tumor-suppressor gene.” Oncogene 9:1703-1711.
Onclercq, R., (1988). “c-myc products trans-activate the adenovirus E4 promoter in EC stem cells by using the same target sequence as E1A products.” J. Virol. 62:4533-4537.
Orten, D., et al. (1994). “Differential effects of monoclonal antibodies on activating transcription factor-1 and cAMP response element binding protein interactions with DNA.” J. Biol. Chem. 269(51):32254-32263.
Pack, P., et al. (1995). “Tetravalent miniantibodies with high avidity assembling in Escherichia coli.” J. Mol. Biol. 246:28-34.
Padlan, E. A., (1976). “Model building studies of antigen binding sites: the hapten binding site of MOPc315.” Cold Spring Harbor Symp. Quant. Biol. 41:627-637.
Page, M., et al. (1988). “Optimization of the tetrazolium-based colorimetric assay for the measurement of cell number and cytoxicity.” British J. Immunopharmacol. 10:785-793.
Pan, S., et al. (1998). Oncogene 16(12):1625-1631.
Pantoliano, M. W., et al. (1991). “Conformational stability, folding, and ligand-binding affinity of single-chain Fv immunoglobulin fragment expressed in Escherichia coli.” Biochemistry 30:10117-10125.
Pappo, A., et al. (1995). “Biology and therapy of pediatric rhabdomyosarcoma”. J. Clin. Oncol. 13:2123-2139.
Park, K., et al. (1994). “The human retinoblastoma susceptibility gene promoter is positively.” J. Biol. Chem. 269:6083-8.
Parkhurst, K. M., et al. (1996). “Simultaneous binding and bending of promoter DNA by the TATA bonding protein: Real time kinetic measurements.” Biochemistry 35:7459-65.
Parkhurst, K. M., et al. (1997). “Mechanism of TBP binding to the TATA element of DNA.” (MS in final preparation).
Parkhurst, K. M., et al. (1994). “Thermodynamic characterization of the cooperativity of 40S complex formation during the initiation of eukaryotic protein synthesis.” Biochemistry 33:15168-177.
Parkhurst, L. J., et al. (1994). “Changes in the end-to-end distance distribution in an oligonucleotide following hybridization.” SPIE Proceedings of the Time-Resolved Laser Spectroscopy in Biochemistry IV 2137:475-83.
Parkhurst, L. J., et al. “Kinetic studies by fluorescence resonance energy transfer employing a double-labeled oligonucleotide: Hybridization to the oligonucleotide complement and to single-stranded DNA.” Biochemistry 34:285-92.
Parkhurst, L. J., et al. (1995). “Donor-acceptor distance distributions in a double-labeled fluorescent oligonucleotide both as a single strand and in duplexes.” Biochemistry 34:293-300.
Paula, Y., et al. (1999). Mol. Cell. Biol. 19(1):594-601.
Peterman, R., et al. (1998). Oncogene 17:603-610.
Pio, F., et al. (1996). “New insights on DNA recognition by ets proteins from the crystal structure of the PU.1 ETS domain DNA complex.” ______ 271(38): 23329-23337.
Raffray, M., et al. (1997). “Apoptosis and Necrosis in Toxicology: a continuum or distinct modes of cell death?” Pharmacol. Ther. 75:153-177.
Remington's Pharmaceutical Sciences, 18th Ed. (1990, Mack Publishing Co., Easton, Pa.)
Rehfuss, R. P., et. al. (1991). J. Biol. Chem. 226:18431-34.
Richardson, J. H., et al. (1995). “Phenotypic knockout of the high-affinity human interleukin 2 receptor by intracellular single-chain antibodies against the ∂ subunit of the receptor.” Pro. Natl. Acad. Sci. USA, 92:3137-3141
Riechmann, L., et al. (1993). “Phage display and selection of a site-directed randomized single-chain antibody Fv fragment for its affinity improvement.” Biochemistry, 32:8848-8855.
Rihs, H. P., et al. (1991). “The rate of nuclear cytoplasmic protein transport is determined by the case in kinase II site flanking the nuclear localization sequence of the SV40 T-antigen.” EMBO. J 10(3):633-639.
Rini, J. M., et al. (1998). “Structural evidence for induced fit as a mechanism for antibody-antigen recognition.” Science 255:959-965.
Robbins, P. D., et al. (1998). “Viral vectors for gene therapy.” Trends in Biotechnology 16:35-40.
Roberts, B., et al. (1987). “The effect of protein context on nuclear location signal function.” Cell 50:465-475.
Rose, G. D., et al. (1985). “Turns in peptides and proteins.” Adv. Prot. Chem. 37:1-109.
Sara, A. S., et al. (1990). Cancer 65:367-374.
Sassone-Corsi, P. (1995). “Transcription factors responsive to cAMP.” Annu. Rev. Cell. Dev. Biol. 11:355-377.
Sassone-Corsi, P. (1998). Int. J. Biochem. Cell Biol. 30(1):27-38.
Sassone-Corsi P, Lamph W W, Kamps M, Verma IM.fos-Associated cellular p39 is related to nuclear transcription factor AP-1. Cell 54:553-560; 1988
Schier, R., et al. (1996). “Identification of functional and structural amino-acid residues by parsimonious mutagenesis.” Gene 169:147-155.
Schiffer, M., et al. (1989). “Structure of a second crystal form of Bence-Jones protein Loc: Strikingly different domain associations in two crystal forms of a single protein.” Biochemistry 28:4066-4072.
Schreiber, J. K., et al. (1984). “Ligand-binding equilibrium and kinetic measurements on the dimeric myoglobin of bysycon canaliculatum and the comparative ligand-binding of diverse non-cooperative heme proteins.” Comp. Biochem. Physiol. A 129-135.
Shenkin, P. S., et al. (1987). “Predicting the antibody hypervariable loop conformation. I. Ensembles of random conformations for ringlike structures.” Biopolymers 26:2053-2085.
Shipman-Appasamy et al.(1991). DNA Seq. 2:181-191.
Sinha et. al. (1983). Tetrahedron Letters 24:297-300.
Snow, M. E., et al. (1986). “Calculation of the three dimensional changes in protein structure due to amino acid substitutions: The variable region of immunoglobulins.” Proteins: Struct. Funct. Genet. 1:267-279.
Speleman, F., et al. (1997) Mod. Pathol. 10(5):496-9.
Stanfield, R. L., et al. (1990). “Crystal structures of an antibody to a peptide and its complex with peptide antigen at 2.8 A.” Science 248:712-719.
Strachen, H. (1994). “Developmental role of PAX.” Current Opinions in Genetic Development 4: 427.
Studier et al. (1990). Methods Enzymol. 185:60-89.
Stura, E. A., et al. (1994). “Applications of the streak seeding technique in protein crystallization.” Journal Cryst. Growth 110:270-282.
Suzuki, et al. (1993). Proc. Natl. Acad. Sci. USA 90:610-14.
Szilvay, A. M., et. al. (1993). Arch. Virol. 131:393-403.
Tsumoto, K., et al. (1994). “Effect of the order of antibody variable regions on the expression of the single-chain HyHEL10 Fv fragment in E. coli and the thermodynamic analysis of its antigen-binding properties.” Biochem. & Biophys. Res. Com. 201(2):546-551.
van Gelder, C., et al. (1994). “A molecular dynamics approach for the generation of complete protein structures from limited coordinate data.” Proteins: Struct. Funct. Genet. 18:174-185.
Vindelov, L. L. (1977). “Flow microfluorometric analysis of nuclear DNA in cells from solid tumors and cell suspension.” Virchows Arch. Cell. Pathol. 24:227-242.
Volker and Bidwell (1986). Manual of Clinical Laboratory Immunology, 3rd Edition, Chapter 17.
Wells, J. A. (1991). Methods in Enzymol. 202:390-411.
Whitlow, M., et al. (1993). “An improved linker for single-chain Fv with reduced aggregation and enhanced proteolytic stability.” Protein Eng. 6(8):989-995.
Whitlow, M., et al. (1994). “Multivalent Fvs: characterizatio of single-chain Fv oligomers and preparation of a bispecific Fv.” Protein Eng. 7(8):1017-1026.
Wilmot, C. M., et al. (1988). “Analysis and prediction of the different types of b-turns in proteins.” J. Molecular Biology 203:221-232.
Winter, G., et al. (1991). “Man-made antibodies.” Nature 349:293-299.
Wiseman, T., et al. (1989). “Rapid measurement of binding constants and heats of binding using a new titration calorimeter.” Anal. Biochem. 179:131-137.
Xie, S., et al. (1997). Oncogene 15:2069-2075.
Yamamoto, K. K, et al. (1988). “Phosphorylation-induced binding and transcriptional effacacy of nuclear factor CREB.” Nature 334:494-498.
Yoshimura, T., et. al. (1990). EMBO J. 9:2537-2542.
Zhao, L. J., et al. (1992). “Human T-cell lymphotropic virus type I (HTLV-I) transcriptional activator, Tax, enhances CREB binding to HTLV-I 21-base-pair repeats by protein-protein interaction.” Proc. Natl. Acad. Sci. USA 89:7070-7074.
Zheng, Q., et al. (1993). “Determining protein loop conformation suing scaling-relaxation techniques.” Protein Science 2:1242-1248.
Zheng, Q., et al. (1994). “Multiple copy sampling in protein loop modeling: Computational efficiency and sensitivity to dihedral angle perturbations.” Protein Science 3:493-506.
Zhumavekov, T., et al. (1995). “Improved version of a human CD1 minigene based vector for T cell-specific expression in transgenic mice.” J. Immunological Methods 185:133-140.

### Patents and Patent Applications

U.S. Pat. No. 55,641,486
U.S. Pat. No. 5,844,096

1-55. (canceled)

56. A method for modulating transcription factor-mediated gene expression comprising exposing said transcription factor to an effective amount of an inhibitory agent which is expressed intracellularly, said inhibitory agent binding to a linker domain of said transcription factor, wherein said linker domain is located adjacent to the DNA binding domain of the transcription factor, wherein the inhibitory agent binds with sufficient binding affinity to modulate transcription of the gene.

57. The method of claim 56, wherein said transcription factor has a DNA-binding domain distinct from an activation domain.

58. The method of claim 56, wherein said modulation comprises dissociation of the transcription factor from the DNA of said gene.

59. The method of claim 56, wherein said modulation comprises inhibiting binding of the transcription factor to the DNA of the gene.

60. The method of claim 56, wherein said transcription factor is b-ZIP transcription factor.

61. The method of claim 56, wherein said transcription factor comprises a helix-loop-helix protein.

62. The method of claim 56, wherein said transcription factor comprises a zinc finger protein.

63. The method of claim 56, wherein said transcription factor is involved in a specific disease process selected from the group consisting of cancer and infectious disease.

64. The method of claim 56, wherein said transcription factor comprises an oncogenic fusion protein with a DNA-binding function.

65. The method of claim 64, wherein said fusion protein is a tumor specific fusion protein.

66. The method of claim 65, wherein said fusion in protein is specific for mesenchymal tumors.

67. The method of claim 64, wherein said fusion protein is encoded by a chromosomal translocation comprising two or more genes or portions of genes.

68. The method of claim 67, wherein one of said genes or said portion involved in the translocation is selected from the group consisting of genes encoding a b-ZIP transcription factor, helix-loop-helix protein and zinc finger protein.

69. The method of claim 64, wherein said fusion protein is EWS/ATF1.

70. The method of claim 56, wherein said inhibitory agent is selected from the group consisting of an antibody, a subcomponent of an antibody, a peptide mimetic, and a non-peptide mimetic.

71. The method of claim 70, wherein said inhibitory agent is an antibody.

72. The method of claim 71, wherein said inhibitory agent is a monoclonal antibody.

73. The method of claim 70, wherein said inhibitory agent is a subcomponent of an antibody.

74. The method of claim 70, wherein said inhibitory agent is a peptide mimetic.

75. The method of claim 70, wherein said inhibitory agent is a non-peptide mimetic.

76. A method for treating an individual having a transcription factor-mediated disease comprising administering to said individual an effective amount of a composition comprising an inhibitory agent which binds to a linker domain of a transcription factor and a pharmaceutically acceptable carrier, wherein said linker domain is located adjacent to the DNA binding domain of the transcription factor, and wherein the inhibitory agent binds with sufficient binding affinity to the transcription factor to modulate transcription, and wherein said composition exhibits a therapeutically useful change in transcription factor-mediated cell behavior.

77. The method of claim 76, wherein said transcription factor mediated disease is a neoplasia selected from the group consisting of leukemias, lymphomas, and sarcomas.

78. The method of claim 76, wherein said transcription factor mediated disease is an infectious disease.

79. The method of claim 76, wherein said composition comprises a vector which expresses scFv4 intracellularly.

80. The method of claim 76, wherein said inhibitory agent is selected from the group consisting of an antibody, a subcomponent of an antibody, a peptide mimetic, and a non-peptide mimetic.

81. The method of claim 80, wherein said inhibitory agent is an antibody.

82. The method of claim 80, wherein said antibody is a monoclonal antibody.

83. The method of claim 80, wherein said inhibitory agent is a subcomponent of an antibody.

84. The method of claim 80, wherein said inhibitory agent is a peptide mimetic.

85. The method of claim 80, wherein said inhibitory agent is a non-peptide mimetic.

86. The method of claim 56, wherein said inhibitory agent is able to enter the nucleus and bind to the linker domain.

87. The method of claim 56, wherein said inhibitory agent is delivered to the nucleus of said cell by infection with a retroviral vector.

88. The method of claim 56, wherein said modulation of transcription factor-mediated gene expression occurs within a cancerous cell.

89. The method of claim 56, wherein said modulation of transcription factor-mediated gene expression occurs within a virally infected cell.

90. The method of claim 88, wherein said cancerous cell is a sarcoma.

91. The method of claim 90, wherein said sarcoma is mesenchymal.

92. The method of claim 88, wherein said cancerous cell is a Clear Cell Sarcoma.

93. The method of claim 73, wherein said subcomponent of an antibody is a short chain variable fragment.

94. The method of claim 93, wherein said short chain variable fragment is scFv4.

95. The method of claim 69, wherein said linker domain comprises amino acids 205-219 of SEQ ID NO: 1.
